(19) United States
(12) Patent Application Publication
(10) Pub. No.: US 2022/0118025 A1
(43) Pub. Date: Apr. 21, 2022

(54) NANOFIBROUS ENCAPSULATION DEVICE FOR SAFE DELIVERY OF THERAPEUTIC AGENTS

(71) Applicant: CORNELL UNIVERSITY, Ithaca, NY (US)

(72) Inventors: Minglin MA, Ithaca, NY (US); Xi WANG, Ithaca, NY (US); James A. FLANDERS, Ithaca, NY (US)

(21) Appl. No.: 17/502,949

(22) Filed: Oct. 15, 2021

Related U.S. Application Data
(60) Provisional application No. 63/092,357, filed on Oct. 15, 2020.

(51) Int. Cl. A61K 35/39; A61K 35/28; A61K 35/33; A61K 45/06; A61K 47/36; A61K 47/34; D04H 3/009; D04H 3/018; D04H 3/03; D04H 3/073

(52) U.S. Cl. CPC A61K 35/39; A61K 35/28; A61K 35/33; A61K 45/06; A61K 47/36; A61K 47/34; D10B 2331/04; D04H 3/018; D04H 3/03; D04H 3/073; D10B 2509/00; D10B 2331/10; D10B 2331/30; D04H 3/009

(57) ABSTRACT

The present disclosure is directed to an implantable therapeutic delivery device. This device comprises a hydrogel core; one or more therapeutic agents suspended within the hydrogel core; and an elongated nanofibrous substrate having proximal and distal ends, said nanofiber substrate having an interior nanofiber wall defining an internal space that extends longitudinally between the proximal and distal ends of the substrate, wherein the hydrogel core comprising the one or more therapeutic agents is positioned within the internal space. The disclosure is also directed to methods of delivering a therapeutic agent to a subject in need thereof that involves implanting the device described herein.

This application claims the benefit of U.S. Provisional Patent Application Ser. No. 63/092,357, filed Oct. 15, 2020, which is hereby incorporated by reference in its entirety.

### FIELD OF THE INVENTION

The present disclosure relates to an implantable nanofiber-enabled therapeutic delivery device and methods of using the same.

### BACKGROUND OF THE INVENTION

The replacement of inadequate β cells has been proposed as a promising therapy for type 1 diabetes (T1D) (Latres et al., “Navigating Two Roads to Glucose Normalization in Diabetes: Automated Insulin Delivery Devices and Cell Therapy, Cell Metab 29:545-563 (2019); and Aguayo-Mazzucato and Bonner-Weir, “Pancreatic β Cell Regeneration as a Possible Therapy for Diabetes,” Cell Metab 27:57-67 (2018)). Clinical trials with intrahepatic allogeneic islet transplantation have shown insulin independence in patients with diabetes (Shapiro et al., “International Trial of the Edmonton Protocol for Islet Transplantation”, New Engl J Medicine 355:1318-1330 (2006); Shapiro et al., “Islet Transplantation in Seven Patients with Type 1 Diabetes Mellitus Using a Glucocorticoid-Free Immunosuppressive Regimen,” New Engl J Medicine 343:230-238 (2000); and Hering et al., “C. I. T. Consortium, Phase 3 Trial of Transplantation of Human Islets in Type 1 Diabetes Complicated by Severe Hypoglycemia,” Diabetes Care 39:1230-1240 (2016)) but factors including the instant blood mediated inflammatory reaction (IBMIR), the side effects of immunosuppressive drugs and the shortage of human islets from cadavers limit application to patients. Stem cell-derived β (SC-β) cells could provide a nearly unlimited supply of cells and therefore holds great promise for treating T1D (Pagliuca et al., “Generation of Functional Human Pancreatic β Cells In Vitro,” Cell 159:428-439 (2014); Rezania et al., “Reversal of diabetes with insulin-producing cells derived in vitro from human pluripotent stem cells,” Nat Biotechnol 32:1121 (2014); Millman et al., “Generation of stem cell-derived β-cells from patients with type 1 diabetes,” Nat Commun 7:11463 (2016); Velazco-Cruz et al., “Acquisition of Dynamic Function in Human Stem Cell-Derived β Cells,” Stem Cell Rep Stem Cell Rep 12:012 (2019); Nair et al., “Recapitulating endocrine cell clustering in culture promotes maturation of human stem-cell-derived β cells,” Nat Cell Biol 21:263-274 (2019); Maxwell et al., “Gene-edited Human Stem Cell-derived β Cells from a Patient With Monogenic Diabetes Reverse Preexisting Diabetes in Mice,” Sci Transl Med 2(540):eaax9106 doi: 10.1126 (2020)). However, the potential risks of immunosuppression and teratoma formation by undifferentiated stem cells remain formidable concerns (Weir and Susan, “Scientific and Political Impediments to Successful Islet Transplantation,” Diabetes 46:1247-1256 (1997); Zahr et al., “Rapamycin Impairs In Vivo Proliferation of Islet Beta-Cells,” Transplantation 84:1576-1583 (2007); and Hentze et al., “Teratoma formation by human embryonic stem cells: Evaluation of essential parameters for future safety studies,” Stem Cell Res 2:198-210 (2009)). Therefore, the delivery of SC-β cells in a retrievable, immunoprotective encapsulation device that is both safe (i.e. prevents any potential cell escape) and functional (i.e. maintains facile mass transfer) may be critical to the clinical success of stem cell-based therapies for T1D.

Alginate microcapsule-based encapsulation systems have been extensively investigated and proven functional in multiple animal models (Veiseh et al., “Size- and shape-dependent foreign body immune response to materials implanted in rodents and non-human primates,” Nat Mater 14:643-651 (2015); Wang et al., “An encapsulation system for the immunoisolation of pancreatic islets,” Nat Biotechnol 15:358 (1997); Vegas et al., “Combinatorial hydrogel library enables identification of materials that mitigate the foreign body response in primates,” Nat Biotechnol 34:345-352 (2016); N. Bray, “Biomaterials: Modified alginates provide a long-term disguise against the foreign body response,” Nat Rev Drug Discov 15(3):158 (2016); Liu et al., “Zwitterionically modified alginates mitigate cellular overgrowth for cell encapsulation,” Nat Commun 10:5262 (2019); Vegas et al., “Long-term glycemic control using polymer-encapsulated human stem cell-derived beta cells in immune-competent mice,” Nat Med 22:306-311 (2016); and Alagpulinsa et al., “Alginate-microencapsulation of Human Stem Cell-derived β Cells With CXCL12 Prolongs Their Survival and Function in Immunocompetent Mice Without Systemic Immunosuppression,” Am J Transplant Official J Am Soc Transplant Am Soc Transpl Surg 19:1930-1940 (2019). However, it is becoming increasingly recognized that the impossibility to ensure complete graft retrieval will hinder their application for SC-β cell delivery in clinical settings. To take advantage of the biocompatibility and immunoprotective property of alginate hydrogels while endowing retrievability, the applicant's lab has recently developed a thread-reinforced alginate fiber for islet encapsulation (TRAFFIC) device (An et al., “Designing a retrievable and scalable cell encapsulation device for potential treatment of type 1 diabetes,” Proc National Acad Sci 115:E263-E272 (2018)). Similar to alginate microcapsules, TRAFFIC maintained cell viability and reversed diabetes in mouse models. Unlike microcapsules, TRAFFIC was completely retrievable using a laparoscopic procedure. However, alginate and hydrogels are intrinsically weak relative to other materials such as elastomers and prone to swelling and even breakage over time. This increases the risk of exposing transplanted cells to the host immune system and allowing undifferentiated cells to escape from the device.

In comparison, polymer-based encapsulation devices such as those made of semi-permeable polytetrafluoroethylene (PTFE) membranes (e.g. the TheraCyte device or the ViaCyte device) (Haller et al., “Macroencapsulated Human iPSC-Derived Pancreatic Progenitors Protect against STZ-Induced Hyperglycemia in Mice,” Stem Cell Rep 12:787-800 (2019); Kumagai-Braesch et al., “The TheraCyte™ Device Protects Against Islet Allograft Rejection in Immunized Hosts,” Cell Transplant 22:1137-1146(10) (2013); Bruin et al., “Maturation and function of human embryonic stem cell-derived pancreatic progenitors in macroencapsulation devices following transplant into mice,” Diabetologia 56:1987-1998 (2013); Robert et al., “Functional Beta Cell Mass from Device-Encapsulated hESC-Derived Pancreatic Endoderm Achieving Metabolic Control,” Stem Cell Rep 10(3):739-750 (2018); Rezania et al., “Enrichment of human embryonic stem cell-derived NKX6.1-expressing pancreatic progenitor cells accelerates the maturation of insulin-secreting cells in vivo,” Stem Cells 31:2432-2442 (2013); and Faleo et al., “Assessment of Immune Isolation of Allogeneic Mouse Pancreatic Progenitor Cells by a Macroencapsulation Device,” Transplantation 100:1211-1218 (2016) are sturdy and can prevent potential cell escape, representing a safer and more translatable strategy for developing cell replacement therapies. However, their small pore sizes (on the order of ˜100 nm) and foreign body reaction (FBR)-induced fibrotic deposition diminish mass transfer that is critical for long-term function. Similarly, a polycaprolactone (PCL) nanoporous membrane formed around sacrificial nanorods was used to form durable encapsulation devices (e.g. the Encellin device) (Chang et al., “Nanoporous Immunoprotective Device for Stem Cell Derived β Cell Replacement Therapy,” Acs Nano 11:7747-7757 (2017), and Nyitray et al., “Polycaprolactone Thin-Film Micro- and Nanoporous Cell-Encapsulation Devices,” Acs Nano 9:5675-5682 (2015)), but the small pore size (on the order of ˜10 nm) and biodegradability of PCL makes the long-term, reliable membrane function less certain, posing a risk for clinical applications. In addition, in many of these devices, extra layers of more rigid membranes, such as polyethylene terephthalate (PET) mesh, are often required to give mechanical support to the devices and maintain their planar shape in vivo. These additional rigid layers may increase the severity of FBR and negatively impact the function. Lastly, these devices have been designed for subcutaneous implantation which is challenging due to the low degree of vasculature and limited oxygen supply in the subcutaneous space (Pepper et al., “A prevascularized subcutaneous device-less site for islet and cellular transplantation,” Nat Biotechnol 33:518-523 (2015)).

### SUMMARY OF THE INVENTION

A first aspect of the disclosure is directed to an implantable therapeutic delivery device. This device comprises a hydrogel core; one or more therapeutic agents suspended within the hydrogel core; and an elongated nanofibrous substrate having proximal and distal ends, said nanofiber substrate having an interior nanofiber wall defining an internal space that extends longitudinally between the proximal and distal ends of the substrate, wherein the hydrogel core comprising the one or more therapeutic agents is positioned within the internal space.

Another aspect of the present disclosure is directed to a method of delivering a therapeutic agent to a subject in need thereof. This method involves implanting the implantable therapeutic delivery device as described herein into the subject.

In some embodiments, the subject in need of treatment thereof, is a subject having diabetes, and the method of delivering a therapeutic agent to the subject involves implanting an implantable therapeutic delivery device into the subject having diabetes.

In another embodiment, the subject in need of treatment thereof is a subject having a bleeding disorder, and the method of delivering a therapeutic agent to the subject involves implanting an implantable therapeutic delivery device as described herein into the subject having the bleeding disorder.

In another embodiment, the subject in need of treatment thereof is a subject having a lysosomal storage disorder, and the method of delivering a therapeutic agent to the subject involves implanting an implantable therapeutic delivery device as described herein into the subject having the lysosomal storage disorder.

In another embodiment, the subject in need of treatment thereof is a subject having a neurological disorder, and the method of delivering a therapeutic agent to the subject involves implanting an implantable therapeutic delivery device as described herein into the subject having the neurological disorder.

In another embodiment, the subject in need of treatment thereof is a subject having cancer, and the method of delivering a therapeutic agent to the subject involves implanting an implantable therapeutic delivery device as described herein into the subject having cancer disorder.

In another embodiment, the subject in need of treatment thereof is a subject having chronic eye disease and the method of delivering a therapeutic agent to the subject involves implanting an implantable therapeutic delivery device as described herein into the subject having a chronic eye disease.

In another embodiment, the subject in need of treatment thereof is a subject having kidney failure and the method of delivering a therapeutic agent to the subject involves implanting an implantable therapeutic delivery device as described herein into the subject having kidney failure.

In another embodiment, the subject in need of treatment thereof is a subject having chronic pain and the method of delivering a therapeutic agent to the subject involves implanting an implantable therapeutic delivery device as described herein into the subject having chronic pain.

Another aspect of the present disclosure is directed to a method of producing a tubular nanofibrous substrate. This method involves preparing a polyurethane solution in a solvent; and electrospinning the polyurethane solution onto a rotating target to produce a tubular nanofibrous substrate.

Reported herein is an intraperitoneal, tubular, nanofiber integrated cell encapsulation (NICE) device that is both safe and functional long term for delivery of insulin-producing cells including human SC-β cells. The device consists of a highly porous and durable nanofibrous skin made by electrospinning a biocompatible medical grade thermoplastic silicone-polycarbonate-urethane (TSPU), and an alginate hydrogel core, which provides additional immunological protection and maintains the separation of islets or SC-β cells within the device. The device can be implanted and retrieved using laparoscopic procedures. The nanofibrous skin contains interconnected pores of tunable size, and the device pores are on the order of ˜1 μm. Live imaging and histological analysis confirmed the continuous containment of encapsulated cells for 5 months; the device restored normoglycemia in diabetic mice for up to 200 days, including when human SC-β cells were used to reverse diabetes in immunodeficient and immunocompetent mice. The scalability and retrievability of the NICE device with human SC-β cells was also demonstrated in dogs. The NICE design may therefore provide a translatable solution to the balance between safety and functionality in developing a stem cell-based cell replacement therapy for T1D.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F depict the design and characterization of the NICE device including mechanical properties, permeability and cell compatibility. (FIG. 1A) Schematics of the device showing the islet-laden hydrogel core surrounded by the nanofibrous skin that prevents cell penetration while allowing maximum mass transfer, along with a photo of nanofibrous tubes with different diameters (from left to right: 0.5, 1, 1.5, 2 and 3 mm) and a scanning electron microscope (SEM) image of the nanofibers. (FIG. 1B) Tensile test (stress-strain curves) of the nanofibrous tubes (n=4). (FIG. 1C and FIG. 1D) Photographs showing the device being stretched more than three times in length (FIG. 1C) and bent without kink (FIG. 1D). (FIG. 1E) Stimulation index of mouse islets (the ratio of insulin secretion in the buffers of high and low glucose concentrations) encapsulated in the device, compared to that of free-floating islets after 1-day and 7-days culture, mean±SD (n=3). (FIG. 1F) Live (green) and dead (red) staining of free-floating islets and islets encapsulated in device after 1-day culture. The data was compared using the two-tailed Student's t-test. Scale bars: 3 mm (FIG. 1A) macroscopic image, 100 μm (FIG. 1F) and 5 μm (FIG. 1A) microscopic image. n.s., non-significant.

FIGS. 2A-2B depict the in vitro analysis of NICE device. (FIG. 2A) A schematic of electrospinning used to make the device. (FIG. 2B) Quantification of live cells in free islets and encapsulated islets following 24 h in vitro culture, mean±SD (n=5). The two-tailed Student's t-test was performed when the data consisted of two groups. n.s., non-significant.

FIGS. 3A-3J depict the biocompatibility of blank (cell-free) devices in different implantation sites in healthy mice for 2 weeks and 4 weeks. (FIG. 3A-H) Blank (cell-free) devices implanted in the ventral S.C. space for two weeks (FIG. 3A) and four weeks (FIG. 3B), in the dorsal S.C. space for two weeks (FIG. 3C) and four weeks (FIG. 3D), in the E.F.P. for two weeks (FIG. 3E) and four weeks (FIG. 3F), and the in I.P. space for two weeks (FIG. 3G) and four weeks (FIG. 3H). In each panel, representative images from left to right are H&E staining, Masson's Trichrome staining and immunofluorescent staining. Myofibroblasts were stained with αSMA (shown in red) and DAPI (shown in blue). (FIG. 3I) Analysis of the thickness of fibrotic layer measured from Masson's Trichrome staining images, mean±SD (n=4). (FIG. 3J) Analysis of the αSMA⁺ cells measured from immunofluorescent staining images, mean±SD (n=4). The data was compared using one-way ANOVA followed by Tukey's test. ****P<0.0001. Scale bars: 100 μm (FIG. 3A through FIG. 3H). S.C., subcutaneous; E.F.P., epididymal fat pad; I.P., intraperitoneal; H&E, hematoxylin and eosin; αSMA, alpha smooth muscle actin.

FIGS. 4A-4C depict the nanofibrous devices of different fiber sizes and chemistries. (FIG. 4A) SEM images of devices made from different concentrations of PU (from left to right: 8%, 10%, 12%, and 14%) and a different polymer (nylon). (FIG. 4B) Fiber sizes at different concentrations of PU, mean±SD (n=4). (FIG. 4C) H&E staining images of retrieved devices (from left to right: 8%, 10%, 12%, and 14% PU and Nylon) after one month of I.P. implantation in C57BL/6 mice. Scale bar: 100 μm (FIG. 4C) and 2 μm (FIG. 4A). SEM, scanning electron microscope; PU, polyurethane; H&E, haemotoxylin and eosin; I.P., intraperitoneal.

FIGS. 5A-5G depict function of NICE device in protecting allogeneic cells. (FIG. 5A) A fluorescent image of C57BL/6 GFP/luciferase-expressing MSC spheroids. (FIG. 5B) A fluorescent image of FVB GFP/luciferase-expressing mouse islets. (FIG. 5C) A fluorescent image of BALB/c GFP/luciferase-expressing 4T1 spheroids. (FIG. 5D and FIG. 5E) Immunofluorescent staining of mouse GFP/luciferase-expressing islets encapsulated in the device for 120 days (red, INS; green, GCG; blue, DAPI). (FIG. 5F) Co-immunofluorescent staining of the mouse GFP/luciferase-expressing islets (red, insulin; green, GFP; blue, DAPI). (FIG. 5G) H&E staining of devices encapsulated with GFP/luciferase-expressing NIT-1 spheroids in healthy C57BL/6 for 2 months. The inset shows a fluorescent image of NIT-1 spheroids before transplantation. Scale bars: 250 μm (FIG. 5A and FIG. 5B), 100 μm (FIG. 5C and FIG. 5G) and 50 μm (FIG. 5D, FIG. 5E and FIG. 5F). GFP, green fluorescent protein; MSC, mesenchymal stem cell; INS, insulin; GCG, glucagon; H&E, haemotoxylin and eosin.

FIGS. 6A-6L show the survival and confinement of different types of cells within the device. (FIG. 6A) Representative bioluminescent images of healthy C57BL/6 mice transplanted with NICE devices encapsulating GFP/luciferase-expressing syngeneic MSC spheroids for up to 150 days (from left to right are day 1, 30, 60, 90, 120 and 150) and after retrieval (far right). (FIG. 6B) Quantitative analysis of the bioluminescence intensity of the device, mean±SD (n=4). (FIG. 6C) A representative bioluminescent image of retrieved device. (FIG. 6D) H&E staining of a retrieved device at day 150. Arrows point to the MSC spheroids. (FIG. 6E) Representative bioluminescent images of healthy C57BL/6 mice transplanted with NICE devices encapsulating FVB GFP/luciferase mouse islets for up to 120 days (from left to right are day 1, 10, 30, 60, 90 and 120) and after retrieval (far right). (FIG. 6F) Quantitative analysis of the bioluminescence intensity of the device, mean±SD (n=6). (FIG. 6G) A bioluminescent image of a retrieved device. (FIG. 6H) H&E staining of a retrieved device. Arrow points to the mouse islets. The top-right corner shows immunofluorescent staining of mouse islets transplanted for 120 days (red, INS; green, GFP; blue, DAPI). (FIG. 6I) Representative bioluminescent images of healthy C57BL/6 mice transplanted with NICE devices encapsulating BALB/c GFP/luciferase 4T1 spheroids for up to 150 days (from left to right are day 1, 30, 60, 90, 120 and 150) and after retrieval (far right). (FIG. 6J) Quantitative analysis of the bioluminescence intensity of the device, mean±SD (n=5). (FIG. 6K) A bioluminescent image of a retrieved device. (FIG. 6L) H&E staining of a retrieved device. Arrow points to the 4T1 cells encapsulated in device for 120 days. The data was compared using one-way ANOVA followed by Tukey's multiple comparisons test. *P<0.05, ***P<0.001, ****P<0.0001, n.s., non-significant. Scale bars: 1 mm (FIG. 6C, FIG. 6G and FIG. 6K), 100 μm (FIG. 6D, FIG. 6H and FIG. 6L). GFP, green fluorescent protein; MSC, mesenchymal stem cell; H&E, hematoxylin and eosin; INS, insulin.

FIGS. 7A-7Q depict the device function with syngeneic, allogeneic and xenogeneic islets in diabetic immunocompetent C57BL/6 mice. (FIG. 7A through FIG. 7C) Non-fasting blood glucose concentrations of the recipient mice transplanted with syngeneic mouse islets (n=17) (FIG. 7A), allogeneic mouse islets (n=24) (FIG. 7B), and xenogeneic rat islets (n=17) (FIG. 7C). Arrows indicate the time points when implants were retrieved from recipients. (FIG. 7D) Body weights of healthy, engrafted and diabetic mice measured for up to 105 days (n=5). (FIG. 7E) Measurement of blood glucose in IPGTT test in different groups on day 30, mean±SD (n=5 for healthy mouse group, n=4 for engrafted groups, n=6 for diabetic mouse group). (FIG. 7F) Quantification of AUC from (FIG. 7E) for different groups on day 30, mean±SD. (FIG. 7G) Measurement of insulin concentration of retrieved devices from engrafted mice in different groups following ex vivo GSIS test, mean±SD (n=4). (FIG. 7H) Measurement of total insulin content of the pancreas in different groups, mean±SD (n=4-5). (FIG. 7I) Bright field image of encapsulated mouse islets retrieved from allogeneic transplantation model on day 40. (FIG. 7J and FIG. 7K) H&E staining and immunofluorescent staining (shown in inset) of syngeneic islets (FIG. 7J) and allogeneic islets (FIG. 7K) from device retrieved after 40 days. (FIG. 7L) H&E staining and immunofluorescent staining (shown in inset) of xenogeneic rat islets from retrieved device after 30 days. (FIG. 7M) H&E staining and immunofluorescent staining (shown in inset) of syngeneic islets from retrieved device after 120 days (red, INS; green, GCG; blue, DAPI). (FIG. 7N) H&E staining and immunofluorescent staining (shown in inset) of allogeneic islets from retrieved device after 170 days (red, INS; green, GCG; blue, DAPI). (FIG. 7O) H&E staining and immunofluorescent staining (shown in inset) of xenogeneic islets from retrieved device after 120 days (red, INS; green, GCG; blue, DAPI). (FIG. 7P) Quantification of the thickness of fibrotic layer in three different groups: autograft, allograft and xenograft at one month. (FIG. 7Q) Percentage of hormone expression (insulin and glucagon) quantified from immunofluorescent staining images of encapsulated islets retrieved from three different groups: autograft, allograft and xenograft. One point represents one islet. The two-tailed Student's t-test was performed when the data consisted of only two groups. The one-way ANOVA followed by Tukey's test was performed for comparing the multi-group data. *P<0.05, **P<0.01, ****P<0.0001, n.s., non-significant. Scale bars: 200 μm (FIG. 7I), 100 μm (FIG. 7K, FIG. 7M, FIG. 7N and FIG. 7O) and 50 μm (FIG. 7J and FIG. 7L). IPGTT, intraperitoneal glucose tolerance test; AUC, area under curve; GSIS, glucose stimulation insulin secretion; H&E, hematoxylin and eosin; INS, insulin; GCG, glucagon.

FIG. 8 depicts the blood glucose measurement in IPGTT test of engrafted recipients at 4 months. IPGTT, intraperitoneal glucose tolerance test.

FIGS. 9A-9D depict the analysis of rodent islets before and after transplantation. Live (green) and dead (red) staining of rodent islets before transplantation (FIG. 9A) and one month after transplantation (FIG. 9B) (from left to right are syngeneic, allogeneic and xenogeneic islets). (FIG. 9C) Quantification of live cells in rodent islets before and after transplantation by calculating intensity of fluorescence in live and dead staining images (n=4). (FIG. 9D) Percentage of hormone expression (insulin and glucagon) quantified from immunofluorescent staining images of rodent islets used in three different groups: autograft, allograft and xenograft before transplantation. One point represents one islet. The two-tailed Student's t-test was performed when the data consisted of two groups. *P<0.05, ***P<0.001, ****P<0.0001, respectively. Scale bars: 200 μm (FIG. 9A and FIG. 9B).

FIGS. 10A-10H depict the immunoprotective function of the device. Analysis of immune cells in the fibrotic layer around the NICE device (FIG. 10A through FIG. 10D) and antibody concentrations (FIG. 10E through FIG. 10H) in three different groups: autograft, allograft and xenograft. (FIG. 10A) Percentage of immune cells and non-immune cells in live cells, mean±SD (n=5). (FIG. 10B) Percentage of CD3⁺ cells in leukocytes, mean±SD (n=5). (FIG. 10C) Percentage of CD4⁺ cells in leukocytes, mean±SD (n=5). (FIG. 10D) Percentage of CD8⁺ cells in leukocytes, mean±SD (n=5). (FIG. 10E) Measurement of mouse total IgG in serum extracted from recipients with autografts (red line), allografts (blue line) and xenografts (orange line) before transplantation and at 1 w, 2 w, 3 w and 4 w post-transplantation, mean±SD (n=5). (FIG. 10F) Measurement of mouse total IgM in serum extracted from recipients with autografts (red line), allografts (blue line) and xenografts (orange line) before transplantation and at 1 w, 2 w, 3 w and 4 w post-transplantation, mean±SD (n=5). (FIG. 10G) Measurement of mouse IgG in retrieved device and in intraperitoneal (I.P.) fluid extracted from recipients after device retrieval, mean±SD (n=4). (FIG. 10H) Measurement of mouse IgM in retrieved device and in I.P. fluid extracted from recipients after device retrieval, mean±SD (n=4). The two-tailed Student's t-test was performed when the data consisted of only two groups. The one-way ANOVA followed by Tukey's test was performed for comparing the multi-group data. *P<0.05, **P<0.01, ****P<0.0001, n.s., non-significant.

FIGS. 11A-11B depict the histological analysis of fibrotic layer surrounding device. Immunofluorescent staining of fibrotic layer around device with CD3 (red) and F4/80 (green) antibodies under two conditions: allograft (FIG. 11A) and xenograft (FIG. 11B) (higher magnifications in the top right corner). Scale bars: 50 μm (FIG. 11A and FIG. 11B). White dash lines show the boundary of the nanofibrous membrane.

FIGS. 12A-12D depict the analysis of immune cells in the fibrotic layer surrounding the NICE device in three different groups: autograft, allograft and xenograft. Percentage of macrophages (FIG. 12A), B cells (FIG. 12B), neutrophils (FIG. 12C) and dendritic cells (FIG. 12D) in leukocytes, mean±SD (n=5). The one-way ANOVA followed by Tukey's test was performed for comparing the multi-group data. *P<0.05, n.s., non-significant.

FIGS. 13A-13B depict the analysis of donor specific antibodies. Analysis of allo-antibodies IgG (FIG. 13A) and IgM (FIG. 13B) in serum extracted from C57BL/6 mice with allogeneic islets encapsulated in NICE devices at day 0 (orange curve) and day 28 (purple curve), in serum extracted from sensitized mice with allogeneic islets transplanted in kidney capsule at day 28 (green curve), and in devices retrieved from mice engrafted with encapsulated allogeneic islets at day 28 (blue curve).

FIGS. 14A-14E depicts the analysis of human islets before transplantation. (FIG. 14A) Live (green) and dead (red) staining of human islets before transplantation. (FIG. 14B and FIG. 14C) Immunofluorescent staining of human islets before transplantation. Co-immunofluorescent staining of insulin (red), glucagon (green) and DAPI (blue) (FIG. 14B). Co-immunofluorescent staining of C-peptide (red), NKX6.1 (green) and DAPI (gray) (FIG. 14C). (FIG. 14D) Percentage of hormone expression of insulin, glucagon and insulin/glucagon before transplantation. Each dot represents one islet (n=24). (FIG. 14E) Percentage of β cell marker expression of C-peptide, NKX6.1 and C-peptide/NKX6.1 before transplantation. Each point represents one islet (n=12). Scale bars: 200 μm (FIG. 14A) and 50 μm (FIG. 14B and FIG. 14C). INS, insulin; GCG, glucagon.

FIGS. 15A-15N depict the device function with human islets in diabetic immunodeficient SCID-beige mice. (FIG. 15A) Bright field image of human islets before transplantation. (FIG. 15B) Measurement of non-fasting blood glucose (n=11). Arrows indicate the time points when implants were retrieved from recipients. (FIG. 15C) Measurement of human C-peptide in serum of the mice at 0 min and 90 mins following IPGTT test after 2, 8 and 14 weeks of transplantation, mean±SD (n=3-4). (FIG. 15D) Typical blood glucose measurement in IPGTT test of engrafted recipients (week 8). (FIG. 15E through FIG. 15G) H&E staining (FIG. 15E) and immunofluorescent staining (FIG. 15F and FIG. 15G) of human islets from retrieved device after 40 days (higher magnifications on the right). (FIG. 15H through FIG. 15J) H&E staining (FIG. 15H) and immunofluorescent staining (FIG. 15I and FIG. 15J) of human islets from retrieved device after 105 days (higher magnifications on the right). (FIG. 15F and FIG. 15I) Co-immunofluorescent staining of insulin (INS, red), glucagon (GCG, green) and DAPI (blue). (FIG. 15G and FIG. 15J) Co-immunofluorescent staining of C-peptide (red), NKX6.1 (green) and DAPI (gray). (FIG. 15K) Percentage of hormone expression (insulin, glucagon and insulin/glucagon) quantified from immunofluorescent staining images. One point represents one islet (n=15). (FIG. 15L) Percentage of 3 cell marker expressions (C-peptide, NKX6.1 and C-peptide/NKX6.1) quantified from immunofluorescent staining images. One point represents one islet (n=15). (FIG. 15M) Measurement of C-peptide concentration of retrieved devices from engrafted mice following ex vivo GSIS test, mean±SD (n=4). (N) Measurements of dynamic normalized Fluo-4 fluorescence intensity for retrieved human islets challenged sequentially with 2, 20, 2 mM glucose and 30 mM KCl, mean SEM (n=11). The two-tailed Student's t-test was performed when the data consisted of two groups. *P<0.05, **P<0.01. Scale bars: 200 μm (FIG. 15A), 100 μm (FIG. 15E and FIG. 15H) and 50 μm (FIG. 15I and FIG. 15J). IPGTT, intraperitoneal glucose tolerance test; H&E, hematoxylin and eosin; INS, insulin; GCG, glucagon; GSIS, glucose stimulation insulin secretion; KCl, potassium chloride.

FIGS. 16A-16T depict the device function with SC-β cells in diabetic immunodeficient NSG mice. (FIG. 16A) Bright field image of SC-β cell aggregates before transplantation. (FIG. 16B and FIG. 16C) Representative flow cytometric dot plots of dispersed stage 6 SC-β cells immunostained for the indicated markers. (FIG. 16D) Dynamic glucose-stimulated human insulin secretion of cells in stage 6 in a perfusion GSIS assay. Data for each individual time point is shown as mean±SEM (n=3). Cells are perfused with low glucose (2 mM) and high glucose (20 mM) as indicated. (FIG. 16E) Oxygen consumption rate of human islets (HI, n=8) and SC-β cells (n=17) at high glucose (20 mM) normalized to DNA content, mean±SD. (FIG. 16F) Measurement of non-fasting blood glucose of the mice transplanted with SC-β cells in the device (n=16). Arrows indicate the time points when implants were retrieved from recipients. (FIG. 16G) Measurement of human C-peptide in mouse serum at 0 min and 90 mins following IPGTT test after 2 and 8 weeks of transplantation, mean±SD (n=3-4). (FIG. 16H) Representative blood glucose measurement in IPGTT test of engrafted recipients (n=3-4). (FIG. 16I) Quantification of AUC from (FIG. 16H) for different groups, mean±SD. (FIG. 16J) Measurement of C-peptide concentration of retrieved devices from engrafted mice following ex vivo GSIS test, mean±SD (n=4). (FIG. 16K) Measurements of dynamic normalized Fluo-4 fluorescence intensity for retrieved SC-β cells challenged sequentially with 2, 20, 2 mM glucose and 30 mM KCl, mean SEM (n=14). (FIG. 16L through FIG. 16N) H&E staining (FIG. 16L) and immunofluorescent staining (FIG. 16M and FIG. 16N) of SC-β cells from retrieved device after 40 days (higher magnification on the right). (FIG. 16O through FIG. 16R) H&E staining (FIG. 16O) and immunofluorescent staining (FIG. 16P through FIG. 16R) of SC-β cells from retrieved device after 120 days. (FIG. 16M and FIG. 16P) Co-immunofluorescent staining of insulin (red), glucagon (green) and DAPI (blue). (FIG. 16N and FIG. 16Q) Co-immunofluorescent staining of C-peptide (red), NKX6.1 (green) and DAPI (gray). (FIG. 16R) High magnification images of cell aggregates from (FIG. 16P) and (FIG. 16Q). (FIG. 16S) Percentage of hormone expression (insulin, glucagon and insulin/glucagon) per cell aggregate quantified from immunofluorescent staining images. One point represents one aggregate (n=44). Samples were collected from four animals. (FIG. 16T) Percentage of β cell marker expressions (C-peptide, NKX6.1 and C-peptide/NKX6.1) per cell aggregate quantified from immunofluorescent staining images. One point represents one aggregate (n=43). Samples were collected from four animals. The two-tailed Student's t-test was performed when the data consisted of two groups. The one-way ANOVA followed by Tukey's test was performed for comparing the multi-group data. *P<0.05, **P<0.01, n.s., non-significant. Scale bars: 250 μm (A), 100 μm (FIG. 16L through FIG. 16Q), 25 μm (FIG. 16R). SC-β, stem cell derived β cell; GSIS, glucose stimulation insulin secretion; IPGTT, intraperitoneal glucose tolerance test; AUC, area under curve; H&E, hematoxylin and eosin; INS, insulin; GCG, glucagon; KCl, potassium chloride.

FIGS. 17A-17H depict the function of NICE device in reversing diabetes using human SC-β cells in immunodeficient mice. (FIG. 17A and FIG. 17B) Immunofluorescent staining of SC-β cells before transplantation. Co-immunofluorescent staining of insulin (red), glucagon (green) and DAPI (blue) (FIG. 17A). Co-immunofluorescent staining of C-peptide (red), NKX6.1 (green) and DAPI (gray) (FIG. 17B). (FIG. 17C) Percentage of hormone expression of insulin, glucagon and insulin/glucagon before transplantation. Each point represents one cluster (n=13). (17D) Percentage of β cell marker expression of C-peptide, NKX6.1 and C-peptide/NKX6.1 before transplantation. Each point represents one cluster (n=13). (FIG. 17E) Static human insulin secretion of cells in stage 6 in a GSIS assay, mean±SD (n=6). (FIG. 17F) Stimulation index in IPGTT at 2 weeks and 8 weeks (the ratio of C-peptide concentration at 90 mins to that at 0 mins), mean±SD (n=3-4). (FIG. 17G) Blood glucose measurement in IPGTT at 8 weeks (n=3). (FIG. 17H) Measurement of total insulin content of the pancreas in different groups, mean±SD (n=4-5). The two-tailed Student's t-test was performed when the data consisted of two groups. The one-way ANOVA followed by Tukey's test was performed for comparing the multi-group data. *P<0.05, ****P<0.0001, n.s., non-significant. Scale bars: 50 μm (FIG. 17A and FIG. 17B). SC-β, stem cell derived β cell; INS, insulin; GCG, glucagon; GSIS, glucose stimulation insulin secretion; IPGTT, intraperitoneal glucose tolerance test.

FIGS. 18A-18N depict the explanted devices from individual recipient mice engrafted with human SC-β cells. (FIG. 18A through FIG. 18C) H&E staining (FIG. 18A) and immunofluorescent staining (FIG. 18B and FIG. 18C) of SC-β cells from device retrieved after 40 days (higher magnification provided below). (FIG. 18D through FIG. 18F) H&E staining (FIG. 18D) and immunofluorescent staining (FIG. 18E and FIG. 18F) of SC-β cells from device retrieved after 50 days. (FIG. 18F) Higher magnification images of (FIG. 18E). (FIG. 18G) H&E staining and immunofluorescent staining of SC-β cells from device retrieved after 60 days. (FIG. 18H and FIG. 18I) Higher magnification images of (FIG. 18G). (FIG. 18J through FIG. 18L) H&E staining (FIG. 18J) and immunofluorescent staining (FIG. 18K and FIG. 18L) (higher magnification shown on the right) of SC-β cells from device retrieved after 85 days. (FIG. 18M and FIG. 18N) Immunofluorescent staining of SC-β cells from device retrieved after 120 days. Co-immunofluorescent staining of insulin (red), glucagon (green) and DAPI (blue) (FIG. 18B, FIG. 18E (top), FIG. 18F (left), FIG. 18G (middle), FIG. 18H, FIG. 18K and FIG. 18M). Co-immunofluorescent staining of C-peptide (red), NKX6.1 (green) and DAPI (gray) (FIG. 18C, FIG. 18E (bottom), FIG. 18F (right), FIG. 18G (right), FIG. 18I, FIG. 18L and FIG. 18N). Scale bars: 100 μm (FIG. 18A, FIG. 18B, FIG. 18D, FIG. 18E, FIG. 18G and FIG. 18J) and 25 μm (FIG. 18C, FIG. 18F, FIG. 18H, FIG. 18I, and FIG. 18K through FIG. 18N). SC-β, stem cell derived β cell; H&E, haemotoxylin and eosin, INS, insulin; GCG, glucagon.

FIGS. 19A-19D depict the analysis of MAFA expression in SC-β cells before and after transplantation. Representative immunofluorescent images of human SC-β cells before transplantation (FIG. 19A) and retrieved from immunodeficient mice (FIG. 19B) and immunocompetent mice (FIG. 19C). (C-peptide, red; MAFA, green and DAPI, gray). White triangles indicate the C-peptide⁺/MAFA⁺ β cells. (FIG. 19D) Percentage of MAFA⁺ cells in different groups (n=5). Scale bars: 25 μm (FIG. 19A through FIG. 19C). The one-way ANOVA followed by Tukey's test was performed for comparing the multi-group data. ****P<0.0001.

FIGS. 20A-20P depict the device function with SC-β cells in xenogeneic mouse and dog models. Device function with SC-β cells in diabetic immunocompetent C57BL/6 mice (FIG. 20A through FIG. 20H), and scalability, retrievability and SC-β cell survival in healthy dogs (I-P). (FIG. 20A) Measurement of non-fasting blood glucose of the mice transplanted with SC-β cells in device (n=16). Arrow indicates the time point when implants were retrieved from recipients. (FIG. 20B) Measurement of human C-peptide in mouse serum at 0 min and 90 mins following IPGTT test after 2 and 4 weeks of transplantation, mean±SD (n=3-4). (FIG. 20C) Representative blood glucose measurement in IPGTT test of engrafted recipients with human SC-β cells (black line), human islets (blue line) and failed human SC-β cells (red line) 14 days post-transplant. (FIG. 20D) Measurement of C-peptide concentration of retrieved devices from engrafted mice following ex vivo GSIS test, mean±SD (n=4). (FIG. 20E) Measurements of dynamic normalized Fluo-4 fluorescence intensity for retrieved SC-β cells challenged sequentially with 2, 20, 2 mM glucose and 30 mM KCl, mean±SEM (n=6). (F-H) H&E staining (FIG. 20F) and immunofluorescent staining (FIG. 20G and FIG. 20H) of SC-β cells from retrieved device after 35 days (higher magnification on the right). Red arrows in (FIG. 20F) indicated cells penetrating from outside to inside of the outer surface of the nanofibrous membrane. (FIG. 20G) Co-immunofluorescent staining of insulin (red), glucagon (green) and DAPI (blue). (FIG. 20H) Co-immunofluorescent staining of C-peptide (red), NKX6.1 (green) and DAPI (gray). (FIG. 20I) Photograph showing the laparoscopic implantation of device (n=3). Red arrows point to the device in a 10 mL pipette during implantation. (FIG. 20J) Laparoscopic image showing the device implanted in I.P. space near the liver in a dog. White arrows point to the device. (FIG. 20K) Laparoscopic images showing the device being pulled out from a dog during retrieval. (FIG. 20L) Photograph of a device before transplantation and a device retrieved from a dog after 2 weeks. (FIG. 20M) Bright field image of encapsulated SC-β cells after retrieval (red arrows point to SC-β cells). (FIG. 20N) Live (green) and dead (red) staining of the SC-β cells in (FIG. 20M). (FIG. 20O and FIG. 20P) H&E staining (FIG. 20O) and immunofluorescent staining (FIG. 20P) of SC-β cells from retrieved device after 2 weeks. (FIG. 20P) Co-immunofluorescent staining of insulin (red), glucagon (green) and DAPI (blue). The two-tailed Student's t-test was performed. **P<0.01, ***P<0.001, n.s., non-significant. Scale bar: 100 μm (FIG. 20F, FIG. 20G, FIG. 20M and FIG. 20N), 50 μm (FIG. 20O) and 25 μm (FIG. 20H and FIG. 20P). SC-β, stem cell derived β cell; IPGTT, intraperitoneal glucose tolerance test; GSIS, glucose stimulation insulin secretion; KCl, potassium chloride; H&E, hematoxylin and eosin; INS, insulin; GCG, glucagon; I.P., intraperitoneal.

FIGS. 21A-21B depict the function of NICE device in reversing diabetes using human islets in immunocompetent mice. (FIG. 21A) Measurement of non-fasting blood glucose of diabetic mice transplanted with human islets in device (n=4). Arrow indicates the time point when implants were retrieved from recipients. (FIG. 21B) Measurement of C-peptide concentration of retrieved devices from engrafted mice following ex vivo glucose stimulation insulin secretion (GSIS) test, mean±SD (n=3). The two-tailed Student's t-test was performed. *P<0.05.

FIG. 22 depicts the thickness of fibrotic layer around the NICE devices with SC-β cells implanted in immunodeficient mice and immunocompetent mice. mean SD (n=6). The two-tailed Student's t-test was performed. ****P<0.0001.

FIGS. 23A-23H depict the function of NICE device in reversing diabetes using human SC-β cells in immunocompetent mice. (A-C) H&E staining (FIG. 23A) and immunofluorescent staining (FIG. 23B and FIG. 23C) of SC-β cells from device retrieved after 30 days. (FIG. 23D and FIG. 23E) H&E staining (FIG. 23D) and immunofluorescent staining (E) of SC-β cells from device retrieved after 30 days. (FIG. 23F through FIG. 23H) H&E staining (FIG. 23F) and immunofluorescent staining (FIG. 23G and FIG. 23H) of SC-β cells from device retrieved after 35 days (higher magnification images on the right). Co-immunofluorescent staining of insulin (red), glucagon (green) and DAPI (blue) (FIG. 23B, FIG. 23D (right), FIG. 23E (left), FIG. 23F (bottom) and FIG. 23G). Co-immunofluorescent staining of C-peptide (red), NKX6.1 (green) and DAPI (gray) (FIG. 23C, FIG. 23E (right), and FIG. 23H). Scale bars: 100 μm (FIG. 23A, FIG. 23D and FIG. 23F) and 25 μm (FIG. 23B, FIG. 23C, FIG. 23E, FIG. 23G and FIG. 23H). H&E, haemotoxylin and eosin; INS, insulin; GCG, glucagon.

FIGS. 24A-24B depict the analysis of antibodies in serum in xenografts. Mouse total IgG concentration (FIG. 24A) and mouse total IgM concentration (FIG. 24B) in serum of recipients with discordant xenograft (human SC-β cells to mouse), before transplantation and 1 w, 2 w, 3 w and 4 w post-transplantation, mean±SD (n=6).

FIG. 25 depicts the function of NICE device in reversing diabetes using human SC-β cells in immunosuppressed immunocompetent mice. Measurement of non-fasting blood glucose of the mice transplanted with SC-β cells in device (n=8). Arrow indicates the time point when implants were retrieved from recipients.

FIGS. 26A-26D depict the implantation and retrieval of NICE device encapsulating human SC-β cells in dogs. (FIG. 26A) Photographs showing the laparoscopic implantation process. Red arrows point to the device in a 10 mL pipette. (FIG. 26B) A laparoscopic image of the implantation process in the intraperitoneal cavity of dog. (FIG. 26C) Photographs of the other two retrieved devices with minimal tissue adhesion after two weeks. (FIG. 26D) H&E staining of dead SC-β cells in retrieved device. Scale bar: 100 μm (FIG. 26D). SC-β, stem cell derived β cell; H&E, haemotoxylin and eosin.

FIGS. 27A-27B depict the Possible reasons for early transplantation failure. (FIG. 27A) Cells penetrating the device along the arrow direction through a sealing defect. (FIG. 27B) Cells penetrating the device through defects on the nanofiber membrane as indicated by the arrow. Scale bars: 50 μm (FIG. 27A and FIG. 27B).

FIG. 28 depicts the flow chart of quality control strategies.

### DETAILED DESCRIPTION OF THE INVENTION

The present disclosure relates to an implantable nanofiber-enabled therapeutic delivery device, methods of producing the delivery devices, and methods of using the same.

A first aspect of the disclosure is directed to an implantable therapeutic delivery device. This device comprises a hydrogel core; one or more therapeutic agents suspended within the hydrogel core; and an elongated nanofibrous substrate having proximal and distal ends, said nanofiber substrate having an interior nanofiber wall defining an internal space that extends longitudinally between the proximal and distal ends of the substrate, wherein the hydrogel core comprising the one or more therapeutic agents is positioned within the internal space of the nanofibrous substrate.

The nanofibrous substrate of the implantable therapeutic delivery device described herein is constructed to meet the following criteria: i) mechanical toughness to ensure long-term stability and safety upon implantation; ii) a tubular form or the like for high surface area and minimally invasive implantation and retrieval; iii) biocompatibility with host tissue (minimal foreign body reaction); iv) sufficiently small pores to prevent cell escape but high porosity to ensure facile mass transfer; and v) low complexity of design to facilitate translation.

In any embodiment, the nanofiber core substrate of the implantable therapeutic delivery device as described herein comprises a robust, biocompatible material that is tunable and elicits a minimal or no foreign body reaction upon implantation. Suitable materials of the nanofiber core substrate include, without limitation, polyurethanes materials. Suitable polyurethanes include, without limitation, thermoplastic polyester urethanes, polyether urethanes, polycaprolactone urethanes, aromatic polyurethane, aliphatic polyurethanes, and combinations thereof. In one embodiment, the nanofibrous substrates comprises thermoplastic silicone-polycarbonate-urethane (TSPU). An exemplary TSPU comprises CarboSil®, a medical grade thermoplastic silicone-polycarbonate-urethane.

Other polymeric materials suitable for producing the nanofibrous substrate of the implantable device, used either alone or in combination with the polyurethane materials described above include, without limitation, nylon, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(F-caprolactone), poly(lactic-co-glycolic acid), poly(1-lactide-co-F-caprolactone), and combinations thereof.

The nanofibrous substrate can be produced via the electrospinning method described herein to produce a substrate structure comprising a plurality of nanofibers assembled into a plurality of non-woven nanofibers layers. The plurality of non-woven nanofiber layers form the interior and exterior walls of the substrate.

In accordance with this embodiment, the nanofibers of the nanofibrous substrate comprise a fiber diameter of less than 500 nm, i.e., less than 450 nm, less than 400 nm, less than 350 nm, less than 300 nm, less than 250 nm, less than 200 nm, less than 150 nm, or less than 100 nm. In any embodiment, the nanofiber diameter is between 200 and 500 nm, e.g., 225 nm to 475 nm, 250 nm to 450 nm, 250 nm to 425 nm, 250 nm to 400 nm, 250 nm to 375 nm, 250 nm to 350 nm, 250 nm to 325 nm, 250 nm to 300 nm. In any embodiment, the average nanofiber diameter of the substrate is about 270 nm.

In any embodiment, the nanofiber composition of the nanofiber core substrate of the implantable therapeutic delivery device as described herein is homogeneous. In other words, the nanofiber composition comprises the same polymeric material, same fiber diameter, and/or same fiber density throughout the plurality of non-woven layers of substrate. In any embodiment, the nanofiber composition of the nanofiber core substrate is heterogeneous, i.e., the nanofiber composition comprises a mixture of polymeric substrate materials, a heterogeneous mix of fiber diameter, and/or heterogeneous mix of fiber density throughout the plurality of non-woven layers of substrate.

The plurality of non-woven nanofiber layers produce an interior wall having an average thickness of between 10 and 1000 μm. The interior wall may, for example, have a thickness of about 1 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm. In any embodiment, the interior wall of the substrate has a thickness ranging from about 20 μm to about 900 μm, from about 30 μm to about 800 μm, from about 40 μm to about 700 μm, from about 50 μm to about 600 μm, from about 60 μm to about 500 μm, from about 70 μm to about 400 μm, from about 80 μm to about 300 μm, from about 90 μm to about 200 μm. In a preferred embodiment, the interior wall of the nanofibrous substrate has an average thickness of 100 μm.

The nanofibrous substrate is a porous substrate which allows for mass transfer of nutrients into the hydrogel core of the device along with transfer of therapeutic agents and metabolic wastes produced by cells within the hydrogel core out of the device. Suitable average pore size, i.e., average pore diameter, for the nanofibrous substrate is about 0.1 μm to about 5 μm. In any embodiment, the average pore size of the nanofibrous substrate is about 0.1 μm; 0.2 μm, 0.3 μm, 0.4 μm, 0.5 μm, 0.6 μm, 0.7 μm, 0.8 μm, 0.9 μm, 1 μm, 1.1 μm; 1.2 μm, 1.3 μm, 1.4 μm, 1.5 μm, 1.6 μm, 1.7 μm, 1.8 μm, 1.9 μm, 2 μm, 2.1 μm; 2.2 μm, 2.3 μm, 2.4 μm, 2.5 μm, 2.6 μm, 2.7 μm, 2.8 μm, 2.9 μm, 3 μm, 3.1 μm; 3.2 μm, 3.3 μm, 3.4 μm, 3.5 μm, 3.6 μm, 3.7 μm, 3.8 μm, 3.9 μm, 4 μm, 4.1 μm; 4.2 μm, 4.3 μm, 4.4 μm, 4.5 μm, 4.6 μm, 4.7 μm, 4.8 μm, 4.9 μm, or 5 μm. In a preferred embodiment, the nanofibrous substrate has an average pore diameter of about 1 μm.

In any embodiment, the interior nanofiber wall of the nanofibrous substrate forms a tube. In any embodiment, the tube is a cylindrical tube and has a tube diameter of about 0.5 mm to about 3 mm. In any embodiment, the cylindrical tube has a diameter of about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, and about 5 mm.

In any embodiment, the implantable therapeutic delivery device described herein has a rupture strain of ≥2.

In any embodiment, the implantable therapeutic delivery device described herein is elastically deformable up to about 4 MPa, up to about 5 MPa, up to about 6 MPa, or up to about 7 MPa under a 0.5 strain. In one embodiment, the device is elastically deformable up to about 5 MPa under a 0.5 strain.

In any embodiment, the implantable therapeutic delivery device described herein the device has an ultimate tensile strength of between 10 MPa and 20 MPa under a strain of greater than 2. In any embodiment, the device has an ultimate tensile strength of between 13 MPa and 17 MPa. Ultimate tensile strength is a measure of the maximum stress that a material can withstand while being stretched or pulled before breaking. In any embodiment, the device has an ultimate tensile strength of up to about 15 MPa under a strain of greater than 2.

In any embodiment, the implantable therapeutic delivery device described herein the device has a Young's modulus, i.e., a measure of how easily the nanofibrous material of the device stretches and deforms, of about 10.0 MPa, about 10.5 MPa, about 11.0 MPa, about 11.5 MPa, about 12.0 MPa, about 12.5 MPa, about 13.0 MPa, about 13.5 MPa when calculated from 10-20% of its stress-strain curve. In one embodiment, the device has a Young's modulus of about 12.6 MPa when calculated from 10-20% of its stress-strain curve.

In accordance with all aspects of the present disclosure, the nanofiber core substrate of the implantable therapeutic delivery device as described herein has a length of 0.5 cm to 1000 m. The nanofiber core substrate may, for example, have a length ranging from about 0.5 cm, 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, 10 m, 20 m, 30 m, 40 m, 50 m, 60 m, 70 m, 80 m, 90 m, 100 m, 200 μm, 300 m, 400 m, 500 m, 600 m, 700 m, 800 m, 900 m to about 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, 10 m, 20 m, 30 m, 40 m, 50 m, 60 m, 70 m, 80 m, 90 m, 100 m, 200 μm, 300 m, 400 m, 500 m, 600 m, 700 m, 800 m, or 900 m, respectively. In any embodiment, the delivery device has a length of about 1 cm, 10 cm, 20 cm, 30 cm, 40 cm, 50 cm, 60 cm, 70 cm, 80 cm, 90 cm, 1 m, 2 m, 3 m, 4 m, 5 m, 6 m, 7 m, 8 m, 9 m, or 10 m. In any embodiment, the nanofiber core substrate has a length of 1 cm to 1 m.

In any embodiment, the nanofiber substrate of the implantable therapeutic delivery device as described herein comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and combinations thereof. In any embodiment, the nanofiber substrate comprises an anti-inflammatory agent to reduce and/or minimize the foreign body response upon implantation. Suitable anti-inflammatory agents include, without limitation, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

The implantable therapeutic delivery device, as described herein, comprises a hydrogel core that is positioned within the internal space of the tubular nanofibrous substrate. The one or more therapeutic agents delivered via the device are suspended within this hydrogel core.

In any embodiment, one or more of the therapeutic agents is an agent released from a preparation of cells suspended in the hydrogel. Accordingly, in any embodiment, the hydrogel is cell growth matrix material. In any embodiment, the cell growth matrix material compromises a synthetic polymer selected from the group consisting of polyethylene glycol (PEG), poly(acrylic acid), poly(ethylene oxide), poly(vinyl alcohol), polyphosphazene, poly(hydroxyethyl methacrylate), triazole-zwitterion hydrogels, poly(sulfobetaine methacrylate), carboxybetaine methacrylate, poly[2-methacryloyloxyethyl phosphorylcholine, N-Hydroxyethyl acrylamide, copolymers thereof, derivatives thereof, and combinations thereof. In any embodiment, the cell growth matrix material compromises a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, and derivatives or combinations thereof.

In any embodiment, the hydrogel core comprises an alginate. In any embodiment, the alginate of the hydrogel core comprises SLG100 alginate. In any embodiment, the hydrogel core comprises a 0.5% to 4% (w/v) alginate solution. In any embodiment, the hydrogel core comprises a 1% to 3% (w/v) alginate solution. In any embodiment, the hydrogel core comprises a 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0% (w/v) alginate solution. In any embodiment, the hydrogel core comprises a 2% (w/v) alginate solution.

In any embodiment, the cell growth hydrogel material further comprises one or more cell factors to enhance cell growth, differentiation, and/or survival of the cells positioned within the hydrogel material. Suitable cell factors include, without limitation glutamine, non-essential amino acids, epidermal growth factors, fibroblast growth factors, transforming growth factor/bone morphogenetic proteins, platelet derived growth factors, insulin growth factors, cytokines, fibronectin, laminin, heparin, collagen, glycosaminoglycan, proteoglycan, elastin, chitin derivatives, fibrin, and fibrinogen, FGF, bFGF, acid FGF (aFGF), FGF-2, FGF-4, EGF, PDGF, TGF-beta, angiopoietin-1, angiopoietin-2, placental growth factor (PlGF), VEGF, PMA (phorbol 12-myristate 13-acetate), and combinations thereof.

In any embodiment, the preparation of cells positioned in the hydrogel core of the implantable therapeutic delivery device as described herein, is a preparation of single cells. In any embodiment, the preparation of a cells is a preparation of cell aggregates. In any embodiment, the preparation of cells is a preparation of single cells and cell aggregates.

In any embodiment, the preparation of cells positioned in the hydrogel core of the implantable therapeutic delivery device is a preparation of primary cells or a preparation of immortalized cells. In any embodiment, the preparation of cells is a preparation of mammalian cells. In any embodiment, the preparation of cells is a preparation of primate cells, rodent cells, canine cells, feline cells, equine cells, bovine cells, and porcine cells. In any embodiment, the preparation of cells is a preparation of human cells.

In any embodiment, the preparation of cells positioned in the hydrogel core of the implantable therapeutic delivery device as described herein is a preparation of stem cells or stem cell derived cells. In any embodiment, the stem cells are pluripotent, multipotent, oligopotent, or unipotent stem cells. In any embodiment, the preparation of stem cells is selected from a preparation of embryonic stem cells, epiblast cells, primitive ectoderm cells, primordial germ cells, and induced pluripotent stem cells.

In any embodiment, the preparation of cells positioned in the hydrogel core of the implantable therapeutic delivery device is a preparation of cells selected from a preparation of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme overexpressed feline kidney epithelial cells, myogenic cells, embryonic stem cell-derived neural progenitor cells, irradiated tumor cells, proximal tubule cells, neural precursor cells, astrocytes, genetically engineered cells.

In any embodiment, the preparation of cells positioned in the hydrogel core of the implantable therapeutic delivery device comprises a cell density of between 1×10³ to 1×10¹⁰ cells/mL. For example, the cell density may range from about 1×10³ cells/mL, 1×10⁴ cells/mL, 1×10⁵ cells/mL, 1×10⁶ cells/mL, 1×10⁷ cells/mL, 1×10⁸ cells/mL, or 1×10⁹ cells/mL up to about 1×10⁴ cells/mL, 1×10⁵ cells/mL, 1×10⁶ cells/mL, 1×10⁷ cells/mL, 1×10⁸ cells/mL, 1×10⁹ cells/mL or 1×10¹⁰ cells/mL, respectively.

In any embodiment, the preparation of cells positioned in the hydrogel core of the implantable therapeutic delivery device as described herein is a preparation comprising islet cells that release insulin and glucagon. In any embodiment, the preparation of insulin producing cells is a preparation of human SC-β cells. In any embodiment, the preparation comprising islet cells and/or SC-β cells is a preparation of human islets and/or SC-β cells, porcine islets and/or SC-β cells, or rodent islets and/or SC-β cells.

In any embodiment, the preparation of cells comprises an islet density between 1×10³ to 6×10⁵ islet equivalents (IEQs)/mL. In any embodiment, the preparation of cells comprises an islet density between 1×10³ to 6×10⁴ islet equivalents (IEQs)/mL. For example, the islet equivalents may range from about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, or 5×10⁵ up to about 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, or 6×10⁵ islet equivalents (IEQs)/mL

In any embodiment, the hydrogel core of the implantable delivery device described herein further comprises a biologically active agent. Suitable biologically active agents include, without limitation, a protein, peptide, antibody or antibody fragment thereof, antibody mimetic, a nucleic acid, a small molecule, a hormone, a growth factor, an angiogenic factor, a cytokine, an anti-inflammatory agent, and any combination thereof. In one embodiment, the biologically active agent is an anti-inflammatory agent. Suitable anti-inflammatory agents are described supra.

In any embodiment, the hydrogel core of the implantable delivery device described herein further comprises an elongated polymeric scaffold that is positioned within and along the length of the hydrogel core. In any embodiment, the elongated polymeric scaffold comprises a rod, tube, or film. In any embodiment, the elongated polymeric scaffold comprises a material selected from the group consisting of silicone, PDMS, rubber, nylon, polyurethane, polysulfone, polyacrylonitrile, polyester such as polyethylene terephthalate and polybutester, polyvinylidene difluoride, polyacrylamide, poly (ethyl methacrylate), poly(methyl methacrylate), polyvinyl chloride, polyoxymethylene, polycarbonate, polypropylene, polyethylene, polybenzimidazole, polyaniline, polystyrene, polyvinylcarbazole, polyamide, poly vinyl phenol, cellulose acetate, polyacrylamide, poly(2-hydroxyethyl methacrylate), polyether imide, poly(ferrocenyldimethylsilane), poly(ethylene-co-vinylacetate), polyethylene-co-vinyl acetate, polyacrylic acid-polypyrene methanol, poly(ethylene-co-vinyl alcohol), polymetha-phenylene isophthalamide, poly(lactic acid), poly(F-caprolactone), poly(lactic-co-glycolic acid), poly(1-lactide-co-F-caprolactone), and combinations thereof.

In any embodiment, the elongated polymeric scaffold comprises an internal fluidic space containing an oxygen carrier. In any embodiment, the oxygen carrier comprises a perfluorinated compound. Suitable perfluorinated compounds include, without limitation, perfluorotributylamine (FC-43), perfluorodecalin, perfluorooctyl bromide, bis-perfluorobutyl-ethene, perfluoro-4-methylmorpholine, perfluorotriethylamine, perfluoro-2-ethyltetrahydrofuran, perfluoro-2-butyltetrahydrofuran, perfluoropentane, perfluoro-2-methylpentane, perfluorohexane, perfluoro-4-isopropylmorpholine, perfluorodibutyl ether, perfluoroheptane, perfluorooctane, and mixtures thereof.

In any embodiment, the elongated polymeric scaffold of the implantable therapeutic delivery device as described herein comprises one or more therapeutic agents selected from the group consisting of therapeutic proteins, peptides, antibodies or fragments thereof, antibody mimetics, and other binding molecules, nucleic acids, small molecules, hormones, growth factors, angiogenic factors, cytokines, anti-inflammatory agents, and combinations thereof. Suitable anti-inflammatory agents are described supra.

In any embodiment, the proximal and distal ends of the nanofibrous substrate of the implantable therapeutic delivery device as described herein are sealed. In any embodiment, the proximal and distal ends of the nanofiber core substrate are sealed by a heat seal, a suture knot, a clamp, a rubber seal, or a screw closure.

In any embodiment, the implantable device described herein comprises one or more contrast agents to facilitate in vivo monitoring of the implanted device placement, location of the implanted device at some time point after implantation, health of the implanted device, deleterious effects on non-target cell types, inflammation, and/or fibrosis. Suitable contrast agents include, without limitation, nanoparticles, nanocrystals, gadolinium, iron oxide, iron platinum, manganese, iodine, barium, microbubbles, fluorescent dyes, and others known to those of skill in the art.

Methods of in vivo monitoring include but are not limited to confocal microscopy, 2-photon microscopy, high frequency ultrasound, optical coherence tomography (OCT), photoacoustic tomography (PAT), computed tomography (CT), magnetic resonance imaging (MRI), single photon emission computed tomography (SPECT), and positron emission tomography (PET). These alone or combined can provide useful means to monitoring the implantable device.

Another aspect of the present disclosure is directed to a method of delivering a therapeutic agent to a subject in need thereof. This method involves implanting any one of the implantable therapeutic delivery device as described herein into the subject.

In some embodiments, the subject in need of treatment thereof, is a subject having diabetes, and the method of delivering a therapeutic agent to the subject involves implanting an implantable therapeutic delivery device into the subject having diabetes.

In accordance with this embodiment, the one or more therapeutic agents of the implantable therapeutic delivery device is insulin, glucagon, or a combination thereof. In any embodiment, the insulin, glucagon, or combination thereof is released from a preparation of cells positioned in the internal space of the nanofiber core substrate. In any embodiment, the preparation of cells comprises a preparation of islets. In any embodiment, the preparation of islets is a preparation of primate islets, rodent islets, canine islets, feline islets, equine islets, bovine islets, or porcine islets. In any embodiment, the preparation of islets is derived from a preparation of stem cells. In any embodiment, the preparation of stem cells is a preparation of pluripotent, multipotent, oligopotent, or unipotent stem cells. In any embodiment, the preparation of stem cells is a preparation comprising embryonic stem cells, epiblast cells, primitive ectoderm cells, primordial germ cells, and induced pluripotent stem cells.

In another embodiment, the subject in need of treatment thereof is a subject having a bleeding disorder, and the method of delivering a therapeutic agent to the subject involves implanting an implantable therapeutic delivery device as described herein into the subject having the bleeding disorder. In accordance with this embodiment, the bleeding disorder can be any bleeding disorder, such as hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency.

In accordance with this embodiment, the one or more therapeutic agents is a blood clotting factor released from a preparation of cells positioned in the internal space of the nanofiber core substrate. In any embodiment, the preparation of cells comprises recombinant myoblasts, mesenchymal stromal cells, endothelial cells, induced pluripotent stem cell derived endothelial cells, induced pluripotent stem cell derived mesenchymal stromal cells, or a combination thereof. In any embodiment, the blood clotting factor is selected from the group consisting of Factor I, Factor II, Factor V, Factor VII, Factor VIII, Factor IX, Factor X, Factor XI, Factor XII, Factor XIII, and combinations thereof.

In another embodiment, the subject in need of treatment thereof is a subject having a lysosomal storage disorder, and the method of delivering a therapeutic agent to the subject involves implanting an implantable therapeutic delivery device as described herein into the subject having the lysosomal storage disorder. In any embodiment, the one or more therapeutic agents is an enzyme released from a preparation of cells positioned in the internal space of the nanofiber core substrate. In any embodiment, the preparation of cells comprises hematopoietic stem cells, fibroblasts, myoblasts, Baby Hamster Kidney (BHK) cells, Chinese Hamster Ovary cells, Human Amniotic Epithelial (HAE) cells, mesenchymal stromal cells, induced pluripotent stem cell derived mesenchymal stromal cells, or combinations thereof. In any embodiment, the enzyme is selected from the group consisting of α-L-iduronidase, Iduronate-2-sulfatase, α-glucuronidase, Arylsulfatase A, alpha-Galactosidase A, and combinations thereof.

In another embodiment, the subject in need of treatment thereof is a subject having a neurological disorder, and the method of delivering a therapeutic agent to the subject involves implanting an implantable therapeutic delivery device as described herein into the subject having the neurological disorder. In any embodiment, neurological disorder is a sensory disorder. In any embodiment, the neurological disorder is selected from the group consisting of Parkinson's disorder, Alzheimer's disease, epilepsy, Huntington's disease, Amyotrophic lateral sclerosis, chronic pain, visual and hearing loss. In any embodiment, the one or more therapeutic agents is a therapeutic molecule released from a preparation of cells positioned in the internal space of the nanofiber core substrate.

In accordance with this embodiment, the preparation of cells comprises choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, and combinations thereof. In any embodiment, the therapeutic molecule is selected from the group consisting of cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof.

In another embodiment, the subject in need of treatment thereof is a subject having cancer, and the method of delivering a therapeutic agent to the subject involves implanting an implantable therapeutic delivery device as described herein into the subject having cancer disorder. In any embodiment, the one or more therapeutic agents is a therapeutic molecule released from a preparation of cells positioned in the internal space of the nanofiber core substrate. In any embodiment, the preparation of cells comprises IL-2-secreting myoblasts, endostatin-secreting cells, Chinese Hamster Ovary cells, and cytochrome P450 enzyme overexpressed feline kidney epithelial cells. In any embodiment, the therapeutic molecule is selected from IL-2, endostatin, cytochrome P450 enzyme, and combinations thereof.

In another embodiment, the subject in need of treatment thereof is a subject having chronic eye disease and the method of delivering a therapeutic agent to the subject involves implanting an implantable therapeutic delivery device as described herein into the subject having a chronic eye disease. In any embodiment, this method further involves administering one or more trophic factors to the subject to protect compromised retinal neurons and to restore neural circuits. In any embodiment, the chronic eye disease is selected from the group consisting of age-related macular degeneration, diabetic retinopathy, retinitis pigmentosa, glaucoma, macular telangiectasia, and combinations thereof.

In accordance with this embodiment, the one or more therapeutic agents is a therapeutic molecule released from a preparation of cells positioned in the internal space of the nanofiber core substrate. In any embodiment, the preparation of cells comprises human retinal pigment epithelium cells, recombinant human retinal pigment epithelium cells, or a combination thereof. In any embodiment, the therapeutic molecule is selected from the group consisting of ciliary neurotrophic factor, antagonists against vascular endothelial growth factor and platelet-derived growth factor, and combinations thereof.

In another embodiment, the subject in need of treatment thereof is a subject having kidney failure and the method of delivering a therapeutic agent to the subject involves implanting an implantable therapeutic delivery device as described herein into the subject having kidney failure. In any embodiment, the one or more therapeutic agents is a therapeutic molecule released from a preparation of cells positioned in the internal space of the nanofiber core substrate. In any embodiment, the preparation of cells comprises renal proximal tubule cells, mesenchymal stem cells, and a combination thereof.

In another embodiment, the subject in need of treatment thereof is a subject having chronic pain and the method of delivering a therapeutic agent to the subject involves implanting an implantable therapeutic delivery device as described herein into the subject having chronic pain. In any embodiment, chronic pain is chronic pain caused by degenerative back and knee, neuropathic back and knee, or cancer. In any embodiment, the one or more therapeutic agents is a therapeutic molecule released from a preparation of cells positioned in the internal space of the nanofiber core substrate. In any embodiment, the preparation of cells comprises chromaffin cells, neural precursor cells, mesenchymal stem cells, astrocytes, and genetically engineered cells, or a combination thereof. In any embodiment, the therapeutic molecule is selected from the group consisting of catecholamine, opioid peptides, enkephalins, and combinations thereof.

In accordance with aspects of the disclosure related to delivering a therapeutic agent to a subject in need thereof, the method of delivering the therapeutic agent involves implanting an implantable therapeutic delivery device as described herein using a laparoscopic procedure. In some embodiments, the therapeutic delivery device is implanted intraperitoneally, percutaneously, or subcutaneously. In some embodiments, implanting the therapeutic delivery device involves suturing the delivery device to a body wall of the subject. In some embodiments, implanting the therapeutic delivery device involves anchoring the delivery device to a body wall of the subject via a transabdominal portal. In some embodiments, implanting the therapeutic delivery device involves wrapping the delivery device in omentum of the subject. In some embodiments, implanting the therapeutic delivery device involves positioning the delivery device in a cavity between the liver and the diaphragm. In some embodiments, implanting the therapeutic delivery device involves anchoring the delivery device to the diaphragm.

In accordance with the methods of delivering a therapeutic agent to a subject in need thereof or treating one of the various conditions as described above, the method further involves retrieving the implantable therapeutic delivery device from the subject when no longer needed or when the device needs replacement. Accordingly, these methods can further involve implanting a replacement implantable therapeutic delivery device after the initial device is retrieved.

Another aspect of the present disclosure is directed to a method of producing a tubular nanofibrous substrate. This method involves preparing a polyurethane solution in a solvent; and electrospinning the polyurethane solution onto a rotating target to produce a tubular nanofibrous substrate.

In accordance with this aspect of the disclosure, the polyurethane solution comprises any of the exemplary polyurethanes as described supra. In one embodiment, the polyurethane solution is a thermoplastic silicone-polycarbonate-urethane (TSPU) solution. In any embodiment, the TSPU is a medical grade TSPU, such as CarboSil®.

In any embodiment, the TSPU solution comprises 5-20% (w/v) TSPU, i.e., the polymer comprises about 5%, 6%, 7% 8%, 9%, 1%, 110%, 12%, 13%, 14%, 15%, 16%, 17%, 18%, 19%, or 20%.

In some embodiments, the solvent comprises a mixture of tetrahydrofuran (THF) and N, N-Dimethylformamide (DMF). In one embodiment, the solvent comprises a 3:2 mixture of THE and DMF. In one embodiment, the solvent comprises THF. In one embodiment, the solvent comprises DMF.

In another embodiment, the solvent is pure HFIP. In any embodiment, the solvent comprises a mixture of HFIP and formic acid. Other suitable organic solvents include, for example, and without limitation, dichloromethane, N,N-dimethyl formamide, ethanol, methanol, or any combination thereof.

In accordance with this embodiment, the electrospinning is carried out at about 10 kV to about 15 kV. In one embodiment, the electrospinning is carried out at about 13 kV. In any embodiment, the polyurethane solution is pumped at a rate of about 0.25 ml/h to about 1 ml/hl. In any embodiment, the polyurethane solution is pumped at a rate of about 0.5 ml/h.

In any embodiment, the polyurethane solution is ejected through a spinneret. In any embodiment, the spinneret has a bore equivalent to a 23-gauge needle. In any embodiment, the spinneret is mounted on a sliding table that reciprocates at a rate of about 5 cm/s. In any embodiment, the working distance between the spinneret and target is fixed at 8 cm to 12 cm. In any embodiment, the working distance between the spinneret and target is fixed at 10 cm. In any embodiment, the rotating target comprises a rotating rod having a diameter of 0.5-3 mm. In any embodiment, the rotating target rotates at 400-500 RPM.

In accordance with this embodiment, the nanofibrous substrate generated using this method comprises electrospun nanofibers, where the electrospun nanofibers comprise a fiber diameter of less than 500 nm as described above. Additionally, the nanofibrous substrate generated in accordance with this method comprises an average wall thickness of between 10 and 1000 nm as described herein, and an average pore size of 0.1 to 5 μm.

### Embodiments

The invention provides also the following non-limiting embodiments.

Embodiment 1 is an implantable therapeutic delivery device comprising: a hydrogel core; one or more therapeutic agents suspended within the hydrogel core; and an elongated nanofibrous substrate having proximal and distal ends, said nanofiber substrate having an interior nanofiber wall defining an internal space that extends longitudinally between the proximal and distal ends of the substrate, wherein the hydrogel core comprising the one or more therapeutic agents is positioned within the internal space.

Embodiment 2 is the device of embodiment 1, wherein the nanofibrous substrate comprises a polyurethane material.

Embodiment 3 is the device of embodiment 2, wherein the polyurethane material is thermoplastic silicone-polycarbonate-urethane (TSPU).

Embodiment 4 is the device of embodiment 3, wherein the TSPU is a CarboSil®.

Embodiment 5 is the device of any one of embodiments 1-4, wherein the nanofibrous substrate comprises a plurality of non-woven nanofiber layers.

Embodiment 6 is the device of any one of embodiments 1-5, wherein nanofibers of the nanofibrous substrate comprise a fiber diameter of less than 500 nm.

Embodiment 7 is the device of embodiment 6, wherein the nanofibers of the nanofibrous substrate comprise a fiber diameter of between 200 and 500 nm.

Embodiment 8 is the device of embodiment 7, wherein the nanofibers of the nanofibrous substrate comprise an average fiber diameter of about 270 nm.

Embodiment 9 is the device of any one of embodiments 1-8, wherein the interior wall of the nanofibrous substrate has an average thickness of between 10 and 1000 μm.

Embodiment 10 is the device of embodiment 9, wherein the interior wall of the nanofibrous substrate has an average thickness of 100 μm.

Embodiment 11 is the device of any one of embodiments 1-10, wherein the nanofibrous substrate comprises pores, said pores having an average diameter of 0.1 μm to 5 μm.

Embodiment 12 is the device of embodiment 11, wherein the nanofibrous substrate has an average pore diameter of 1 μm.

Embodiment 13 is the device of any one of embodiments 1-12, wherein the device has a rupture strain of ≥2.

Embodiment 14 is the device of any one of embodiments 1-13, wherein the device is elastically deformable up to about 5 MPa under a 0.5 strain.

Embodiment 15 is the device of any one of embodiments 1-14, wherein the device has an ultimate tensile strength of up to about 15 MPa under a strain of greater than 2.

Embodiment 16 is the device of any one of embodiments 1-15, wherein the device has a Young's modulus of about 12.6 MPa when calculated from 10-20% of its stress-strain curve.

Embodiment 17 is the device of any one of embodiments 1-16, wherein the one or more therapeutic agents are secreted from a preparation of cells suspended in the hydrogel core.

Embodiment 18 is the device of embodiment 17, wherein the preparation of cells is a preparation of single cells or a preparation of cell aggregates.

Embodiment 19 is the device of embodiment 17 or embodiment 18, wherein the preparation of cells is a preparation of primary cells or a preparation of immortalized cells.

Embodiment 20 is the device of any one of embodiments 17-19, wherein the preparation of cells is a preparation of mammalian cells.

Embodiment 21 is the device of any one of embodiments 17-20, wherein the preparation of cells is selected from the group consisting of a preparation of primate cells, rodent cells, canine cells, feline cells, equine cells, bovine cells, and porcine cells.

Embodiment 22 is the device of any one of embodiments 17-21, wherein the preparation of cells is a preparation of human cells.

Embodiment 23 is the device of any one of embodiments 17-22, wherein the preparation of cells is a preparation of stem cells or stem cell derived cells.

Embodiment 24 is the device of embodiment 23, wherein the stem cells are pluripotent, multipotent, oligopotent, or unipotent stem cells.

Embodiment 25 is the device of embodiment 23, wherein the preparation of stem cells is selected from the group consisting of embryonic stem cells, epiblast cells, primitive ectoderm cells, primordial germ cells, and induced pluripotent stem cells.

Embodiment 26 is the device of any one of embodiments 17-22, wherein the preparation of cells is a preparation of cells selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells.

Embodiment 27 is the device of any one of embodiments 1-17, wherein the one or more therapeutic agents is selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and any combination thereof.

Embodiment 28 is the device of embodiment 27, wherein the one or more therapeutic agents is insulin and/or glucagon secreted from a preparation of cells suspended in the hydrogel core.

Embodiment 29 is the device of embodiment 28, wherein the preparation of cells is a preparation of islet cells.

Embodiment 30 is the device of embodiment 29, wherein the preparation of cells is a preparation of human SC-β cells.

Embodiment 31 is the device of embodiment 29 or embodiment 30, wherein the preparation is a preparation of human cells, porcine cells, or rodent cells.

Embodiment 32 is the device of embodiment 29, wherein the preparation of islet cells comprises an islet density of about 1×10³ to about 6×10⁴ islet equivalents (IEQs)/mL.

Embodiment 33 is the device of any one of embodiments 17-32, wherein the preparation of cells comprises a cell density of about 1×10³ to about 6×10¹⁰ cells/mL.

Embodiment 34 is the device of any one of embodiments 1-33, wherein the hydrogel core further comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and any combination thereof.

Embodiment 35 is the device of embodiment 34, wherein the anti-inflammatory agent is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

Embodiment 36 is the device of any one of embodiments 1-35, wherein the hydrogel core comprises an alginate.

Embodiment 37 is the device of any one of embodiments 1-36, wherein the hydrogel core comprises SLG100 alginate.

Embodiment 38 is the device of any one of embodiments 1-37, wherein the hydrogel core comprises a 0.5% to 4% (w/v) alginate solution.

Embodiment 39 is the device of any one of embodiments 1-37, wherein the hydrogel core comprises a 2% (w/v) alginate solution.

Embodiment 40 is the device of any one of embodiments 1-39, wherein the interior nanofiber wall of the nanofibrous substrate forms a tube, said tube having a diameter of about 0.5 mm to about 3 mm.

Embodiment 41 is the device of embodiment 40, wherein the nanofiber substrate forms a cylindrical tube.

Embodiment 42 is the device of any one of embodiments 1-41, wherein the proximal and distal ends of the nanofibrous substrate of the device are heat sealed.

Embodiment 43 is a method of delivering a therapeutic agent to a subject in need thereof, said method comprising: implanting the implantable therapeutic delivery device according to any one of embodiments 1-42 into the subject.

Embodiment 44 is a method of treating diabetes in a subject, said method comprising: implanting the implantable therapeutic delivery device according to any one of embodiments 1-42 into the subject having diabetes.

Embodiment 45 is the method of embodiment 44, wherein the one or more therapeutic agents of the implantable therapeutic delivery device is insulin, glucagon, or a combination thereof released from a preparation of cells suspended in the hydrogel core of said device.

Embodiment 46 is the method of embodiment 45, wherein the preparation of cells comprises a preparation of islets.

Embodiment 47 is the method of embodiment 46, wherein the preparation of islets is a preparation of primate islets, rodent islets, canine islets, feline islets, equine islets, bovine islets, or porcine islets.

Embodiment 48 is the method of embodiment 46, wherein the preparation of islets is derived from a preparation of stem cells.

Embodiment 49 is the method of embodiment 48, wherein the preparation of stem cells is a preparation of pluripotent, multipotent, oligopotent, or unipotent stem cells.

Embodiment 50 is the method of embodiment 48, wherein the preparation of stem cells is selected from the group consisting of embryonic stem cells, epiblast cells, primitive ectoderm cells, primordial germ cells, and induced pluripotent stem cells.

Embodiment 51 is a method of treating a bleeding disorder in a subject, said method comprising: implanting the implantable therapeutic delivery device of any one of embodiments 1-42 into the subject having a bleeding disorder.

Embodiment 52 is the method of embodiment 51, wherein the bleeding disorder is selected from the group consisting of hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency.

The method of embodiment 53, wherein the one or more therapeutic agents is a blood clotting factor released from a preparation of cells suspended in the hydrogel core of said device.

Embodiment 54 is the method of embodiment 53, wherein the preparation of cells comprises recombinant myoblasts, mesenchymal stromal cells, endothelial cells, induced pluripotent stem cell derived endothelial cells, induced pluripotent stem cell derived mesenchymal stromal cells, or a combination thereof.

Embodiment 55 is the method of embodiment 53, wherein the blood clotting factor is selected from the group consisting of Factor I, Factor II, Factor V, Factor VII, Factor VIII, Factor IX, Factor X, Factor XI, Factor XII, Factor XIII, and combinations thereof.

Embodiment 56 is a method of treating a lysosomal storage disease in a subject, said method comprising: implanting the implantable therapeutic delivery device of any one of embodiments 1-42 into the subject having the lysosomal storage disease.

Embodiment 57 is the method of embodiment 56, wherein the one or more therapeutic agents is an enzyme released from a preparation of cells suspended in the hydrogel core of said device.

Embodiment 58 is the method of embodiment 57, wherein the preparation of cells comprises hematopoietic stem cells, fibroblasts, myoblasts, Baby Hamster Kidney (BHK) cells, Chinese Hamster Ovary cells, Human Amniotic Epithelial (HAE) cells, mesenchymal stromal cells, induced pluripotent stem cell derived mesenchymal stromal cells or combinations thereof.

Embodiment 59 is the method of embodiment 57, wherein the enzyme is selected from the group consisting of α-L-iduronidase, Iduronate-2-sulfatase, α-glucuronidase, Arylsulfatase A, alpha-Galactosidase A, and combinations thereof.

Embodiment 60 is a method of treating a neurological disorder in a subject, said method comprising: implanting the implantable therapeutic delivery device of any one of embodiments 1-42 into the subject having the neurological disorder.

Embodiment 61 is the method of embodiment 60, wherein the neurological disorder is a sensory disorder.

Embodiment 62 is the method of embodiment 61, wherein the neurological disorder is selected from the group consisting of Parkinson's disorder, Alzheimer's disease, epilepsy, Huntington's disease, Amyotrophic lateral sclerosis, chronic pain, visual loss, hearing loss, peripheral nerve injury, and spinal cord injury.

Embodiment 63 is the method of embodiment 60, wherein the one or more therapeutic agents is a therapeutic molecule released from a preparation of cells suspended in the hydrogel core of said device.

Embodiment 64 is the method of embodiment 63, wherein the preparation of cells comprises choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, myogenic cells, embryonic stem cell-derived neural progenitor cells, and combinations thereof.

Embodiment 65 is the method of embodiment 60, wherein the therapeutic molecule is selected from the group consisting of cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof.

Embodiment 66 is a method of treating a cancer in a subject, said method comprising: implanting the implantable therapeutic delivery device of any one of embodiments 1-42 into the subject having cancer.

Embodiment 67 is the method of embodiment 66, wherein the one or more therapeutic agents is a therapeutic molecule released from a preparation of cells suspended in the hydrogel core of said device.

Embodiment 68 is the method of embodiment 67, wherein the preparation of cells comprises IL-2-secreting myoblasts, endostatin-secreting cells, Chinese Hamster Ovary cells, and cytochrome P450 enzyme overexpressed feline kidney epithelial cells, irradiated tumor cells, and combinations thereof.

Embodiment 69 is the method of embodiment 68, wherein the therapeutic molecule is selected from IL-2, endostatin, cytochrome P450 enzyme, tumor antigens, a cytokine, and combinations thereof.

Embodiment 70 is a method of treating a chronic eye disease in a subject, said method comprising: implanting the implantable therapeutic delivery device of any one of embodiments 1-42 into the subject having a chronic eye disease.

Embodiment 71 is the method of embodiment 70 further comprising: administering one or more trophic factors to the subject to protect compromised retinal neurons and to restore neural circuits.

Embodiment 72 is the method of embodiment 70, wherein the chronic eye disease is selected from the group consisting of age-related macular degeneration, diabetic retinopathy, retinitis pigmentosa, glaucoma, macular telangiectasia, and combinations thereof.

Embodiment 73 is the method of embodiment 70, wherein the one or more therapeutic agents is a therapeutic molecule released from a preparation of cells suspended in the hydrogel core of said device.

Embodiment 74 is the method of embodiment 73, wherein the preparation of cells comprises human retinal pigment epithelium cells, recombinant human retinal pigment epithelium cells, or a combination thereof.

Embodiment 75 is the method of embodiment 73, wherein the therapeutic molecule is selected from the group consisting of ciliary neurotrophic factor, antagonists against vascular endothelial growth factor and platelet-derived growth factor, and combinations thereof.

Embodiment 76 is a method of treating a kidney failure in a subject, said method comprising: implanting the implantable therapeutic delivery device of any one of embodiments 1-42 into the subject having a kidney failure.

Embodiment 77 is the method of embodiment 76, wherein the one or more therapeutic agents is a therapeutic molecule released from a preparation of cells suspended in the hydrogel core of said device.

Embodiment 78 is the method of embodiment 77, wherein the preparation of cells comprises renal proximal tubule cells, mesenchymal stromal cells, endothelial cells, and a combination thereof.

Embodiment 79 is a method of treating a chronic pain in a subject, said method comprising: implanting the implantable therapeutic delivery device of any one of embodiments 1-42 into the subject having a chronic pain.

Embodiment 80 is the method of embodiment 79, wherein the chronic pain is chronic pain caused by degenerative back and knee, neuropathic back and knee, or cancer.

Embodiment 81 is the method of embodiment 79, wherein the one or more therapeutic agents is a therapeutic molecule released from a preparation of cells suspended in the hydrogel core of said device.

Embodiment 82 is the method of embodiment 81, wherein the preparation of cells comprises chromaffin cells, neural precursor cells, mesenchymal stromal cells, astrocytes, and genetically engineered cells, or a combination thereof.

Embodiment 83 is the method of embodiment 81, wherein the therapeutic molecule is selected from the group consisting of catecholamine, opioid peptides, enkephalins, and combinations thereof.

Embodiment 84 is the method according to any one of embodiments 43-83, wherein said implanting is carried out via a laparoscopic procedure.

Embodiment 85 is the method according to any one of embodiments 43-83, wherein said therapeutic delivery device is implanted intraperitoneally, percutaneously, or subcutaneously.

Embodiment 86 is the method according to any one of embodiments 43-83, wherein said implanting involves suturing the delivery device to a body wall of the subject.

Embodiment 87 is the method according to any one of embodiments 43-83, wherein said implanting involves anchoring the delivery device to a body wall of the subject via a transabdominal portal.

Embodiment 88 is the method according to any one of embodiments 43-83, wherein said implanting involves wrapping the delivery device in omentum of the subject.

Embodiment 89 is the method according to any one of embodiments 43-83, wherein said implanting involves positioning the delivery device in a cavity between the liver and the diaphragm.

Embodiment 90 is the method according to any one of embodiments 43-83, wherein said implanting involves anchoring the delivery device to the diaphragm.

Embodiment 91 is the method according to any one of embodiments 43-90, wherein said method further comprises: retrieving the implantable therapeutic delivery device from the subject.

Embodiment 92 is the method according to embodiment 91, wherein said method further comprises: implanting a replacement implantable therapeutic delivery device after said retrieving.

Embodiment 93 is a method of producing a tubular nanofibrous substrate, the method comprising: preparing a polyurethane solution in a solvent; and electrospinning the polyurethane solution onto a rotating target to produce a tubular nanofibrous substrate

Embodiment 94 is a method of embodiment 93, wherein the polyurethane solution is a thermoplastic silicone-polycarbonate-urethane (TSPU) solution.

Embodiment 95 is the method of embodiment 94, wherein the TSPU solution comprises 5-20% (w/v) TSPU.

Embodiment 96 is the method of any one of embodiments 93-95, wherein the solvent comprises a 3:2 mixture of THE and DMF.

Embodiment 97 is the method of any one of embodiments 93-96, wherein electrospinning is carried out at 13 kV.

Embodiment 98 is the method of any one of embodiments 93-97, wherein the polyurethane solution is pumped at a rate of 0.5 ml/h.

Embodiment 99 is the method of any one of embodiments 93-98, wherein the polyurethane solution is ejected through a spinneret.

Embodiment 100 is the method of embodiment 99, wherein the spinneret has a bore equivalent to a 23-gauge needle.

Embodiment 101 is the method of embodiments 99 and 100, wherein the spinneret is mounted on a sliding table that reciprocates at a rate of 5 cm/s.

Embodiment 102 is the method of embodiment 101, wherein a working distance between the spinneret and target is fixed at 10 cm.

Embodiment 103 is the method of any one of embodiments 93-102, wherein the rotating target comprises a rotating rod having a diameter of 0.5-3 mm.

Embodiment 104 is the method of any one of embodiments 93-103, wherein the rotating target rotates at 400-500 RPM.

Embodiment 105 is the method of any one of embodiments 94-103, wherein the TSPU is a CarboSil®.

Embodiment 106 is the method of any one of embodiments 93-105, wherein the nanofibrous substrate comprises electrospun nanofibers.

Embodiment 107 is the method of embodiment 106, wherein the electrospun nanofibers comprise a fiber diameter of less than 500 nm.

Embodiment 108 is the method of embodiment 107, wherein the electrospun nanofibers comprise a fiber diameter of between 200 and 500 nm.

Embodiment 109 is the method of embodiment 107, wherein the electrospun nanofibers comprise an average fiber diameter of about 270 nm.

Embodiment 110 is the method of any one of embodiments 93-109, wherein the nanofibrous substrate has an average wall thickness of between 10 and 1000 nm.

Embodiment 111 is the method of embodiment 110, wherein the nanofibrous substrate has an average wall thickness of 100 nm.

Embodiment 112 is the method of any one of embodiments 93-111, wherein the nanofibrous substrate has pores, wherein the pores have an average pore size of 0.1 to 5 μm.

Embodiment 113 is the method of embodiment 112, wherein the nanofibrous substrate has pores, wherein the pores have an average pore size of 1 μm.

### EXAMPLES

### Example 1—Design, Fabrication and Characterization of the NICE Device

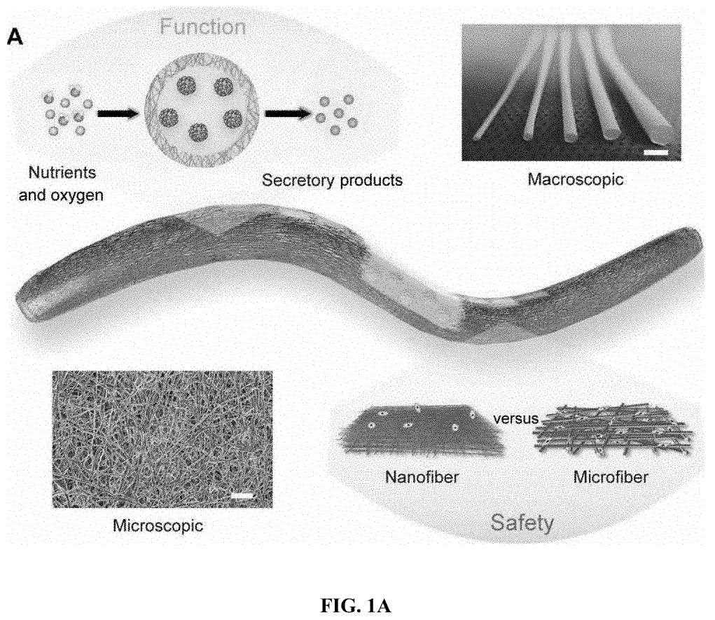

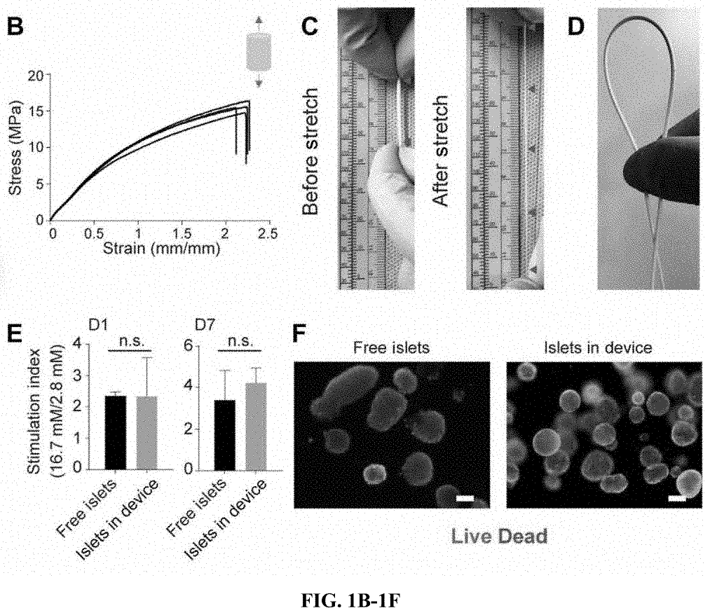

The NICE device was designed to meet several criteria: i) mechanical toughness to ensure long-term stability and safety; ii) tubular form for high surface area and minimally invasive implantation and retrieval; iii) biocompatibility with host tissue (minimal foreign body reaction); iv) sufficiently small pores to prevent cell escape but high porosity to ensure facile mass transfer; and v) low complexity of design to facilitate translation (FIG. 1A). The applicants chose a biocompatible medical grade thermoplastic silicone-polycarbonate-urethane (TSPU) as the polymer and used electrospinning to fabricate the device due to its ability to produce tough, nanofibrous membranes with tunable and interconnected pore structures.

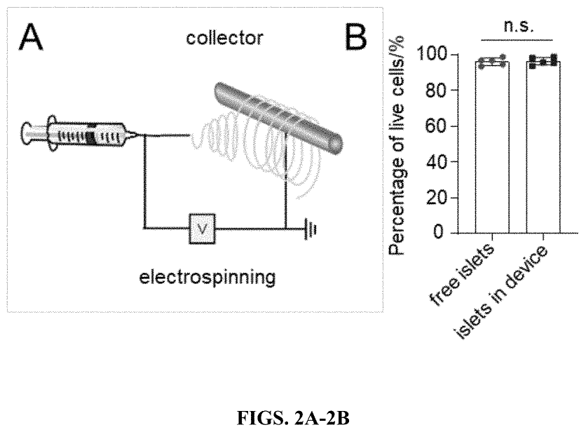

Nanofibers were collected on a rotating, sugar-coated mandrel template (FIG. 2) and nanofibrous tubes were obtained after dissolution of the sugar coating. The tubes had inner diameters ranging from 500 μm to 3 mm and a wall thickness of ˜100 μm (FIG. 1A). Examination using a scanning electron microscope (SEM) verified the non-woven fibrous nature of the NICE membrane with an average fiber diameter of 270 nm (FIG. 1A). Tensile tests demonstrated that the nanofibrous tube remained elastically deformable up to about 5 MPa under a 0.5 strain. The ultimate tensile strength was about 15 MPa at a device strain greater than 2. The Young's modulus calculated from 10%-20% of the curve is ˜12.6±0.6 MPa (n=4) (FIG. 1). Qualitatively, the device can be stretched more than three times its original length (FIG. 1C), bent without kinks (FIG. 1D) and handled using forceps without damage.

Knowing the mechanical strength of the device, the mass transfer properties and compatibility with islets was investigated. A static glucose stimulated insulin secretion (GSIS) test showed that mouse islets encapsulated in the NICE device were capable of responding to low and high glucose, with a mean stimulation index (SI) (insulin secretion at high glucose to that at low glucose) of 2.33 and 4.23 after 1 day and 7 days of culture, respectively (FIG. 1E). The SI of islets cultured inside the device was not significantly different from that of freely floating islets after 1 day (2.33±1.23 vs. 2.34±0.13) (P=0.9888) or 7 days (4.23±0.72 vs. 3.39±1.44) (P=0.5794) of culture (FIG. 1E). The live/dead staining and corresponding quantitative analysis indicated similar viability (˜95%) of islets inside the device to free islets after 24 h culture (FIG. 1F and FIG. 2B). These in vitro experiments demonstrated that the NICE device met the basic requirements as a cell delivery vehicle, providing sufficient mass transfer and cell compatibility to ensure the function of islets.

### Example 2—Biocompatibility of the NICE Device in the Intraperitoneal Space of Mice

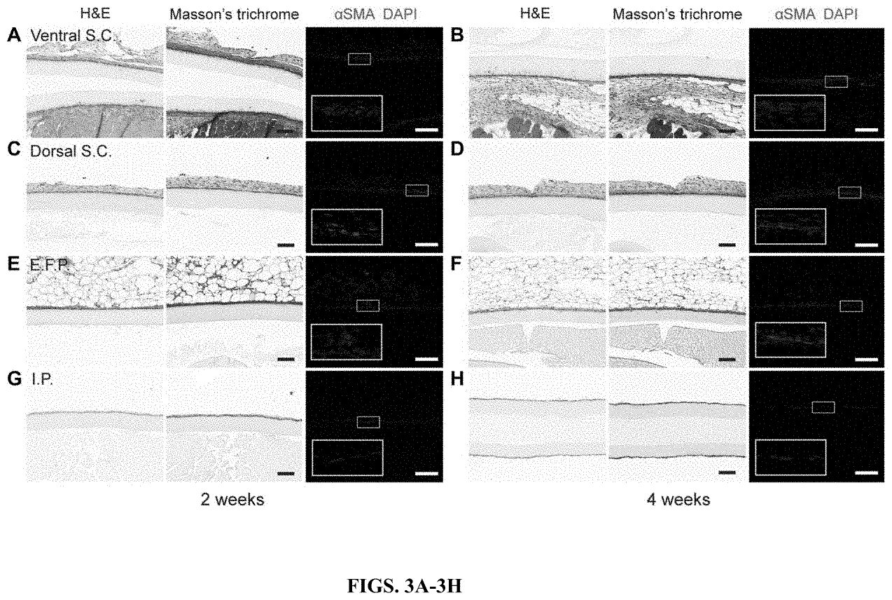

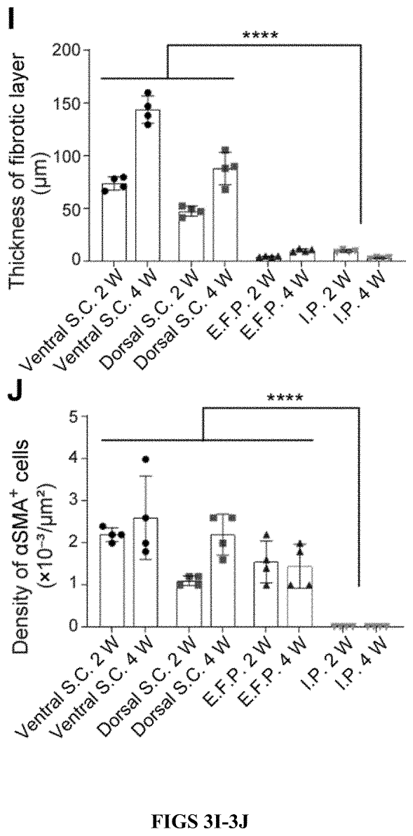

The in vivo biocompatibility of an encapsulation device is an important factor; foreign body reaction (FBR)-induced fibrotic deposition can diminish the mass transfer and affect the function of encapsulated cells. The inventors implanted and compared the FBR to the blank (cell-free) NICE devices in three clinically relevant transplant sites: the intraperitoneal (I.P.) space, the epididymal fat pad (E.F.P.) (as a model of the omentum in large mammals) and the subcutaneous (S.C.) space in C57BL/6 mice. Haemotoxylin and eosin (H&E) staining and Masson's Trichrome staining demonstrated that the devices implanted in either the ventral or dorsal S.C. space formed a thick fibrotic capsule (over 40 μm) (FIGS. 3A to 3D and 3I). The thickness of the fibrotic layer at the 4-week time point was significantly (P<0.0001) increased compared to that at 2 weeks in both the dorsal S.C. space (87.7±13.2 vs. 47.3±4.1 μm) and ventral S.C. space (143.5±11.3 vs. 73.6±5.4 μm) (FIG. 3I). In contrast, devices implanted in the I.P. space or the E.F.P. had much thinner cellular overgrowth (<˜10 μm) than in the S.C. space (FIGS. 3E to 3H and 3I). Although there was no statistically significant difference between the I.P. space and E.F.P. at either 2 weeks (P=0.9754) or 4 weeks (P=0.8610), the cellular overgrowth on the device in the I.P. space at 4 weeks was only a single layer of cells with thickness of 2.9±0.6 μm. Devices implanted in the E.F.P. or the S.C. space were wrapped tightly within tissue. It was noted that the nanofibrous membranes remained impenetrable to cells in all locations.

Myofibroblasts that are present in fibrotic capsules are identifiable by their expression of alpha smooth muscle actin (αSMA). These cells can arise from cell populations that enter the vicinity of a foreign material such as macrophages (Mooney et al., “Cellular Plasticity of Inflammatory Myeloid Cells in the Peritoneal Foreign Body Response,” Am J Pathology 176:369-380 (2010), which is hereby incorporated by reference in its entirety) and may be of bone marrow origin (Campbell et al., “Haemopoietic Origin of Myofibroblasts Formed in the Peritoneal Cavity in Response to a Foreign Body,” J Vasc Res 37:364-371 (2000), which is hereby incorporated by reference in its entirety). The number and density of myofibroblasts trended with the fibrotic layer thickness (FIGS. 3I and 3J). More αSMA+ cells were observed in the S.C. space than in the I.P. space. Interestingly, there was no expression of αSMA in the I.P. group and no foreign body giant cells (FBGCs) were found adjacent to the implant surface. Thus, the FBR was determined to be the least severe in the I.P. space, motivating the choice to investigate this implantation site in this study.

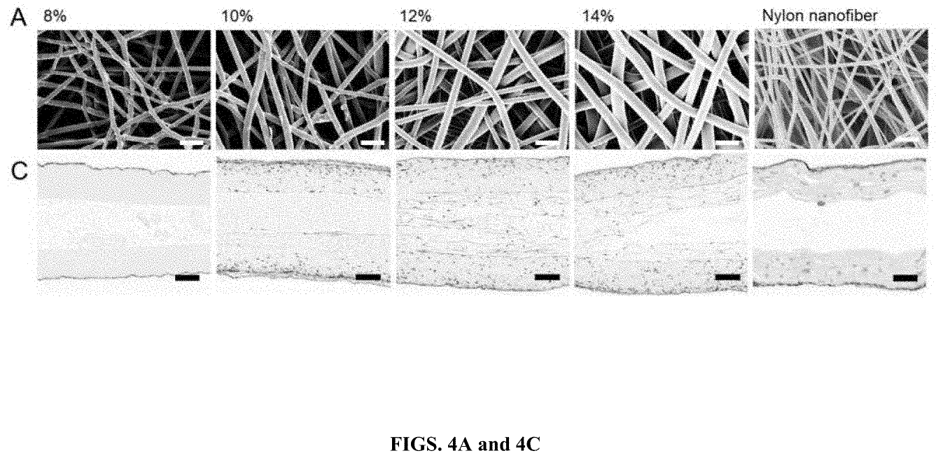

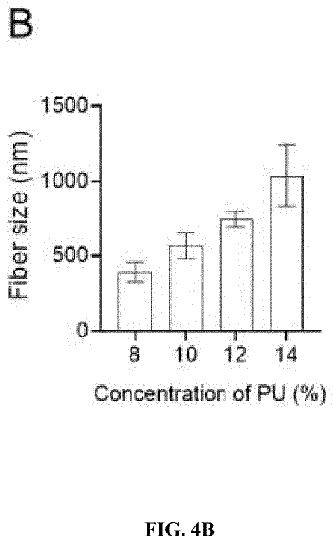

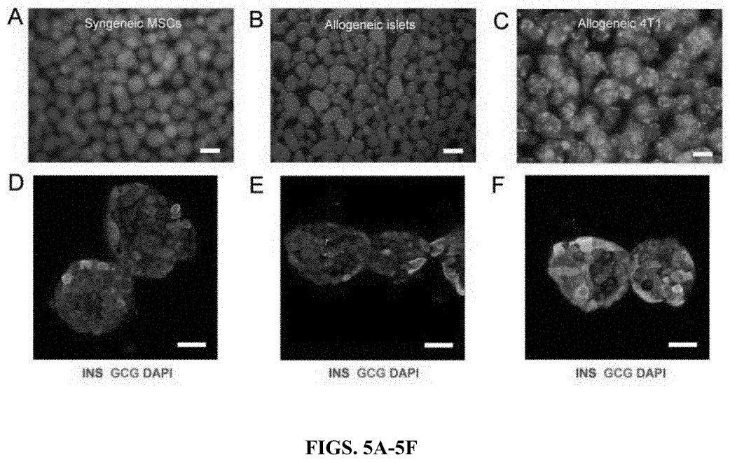

It was hypothesized that the low FBR was due to unique properties of TSPU and the small fiber size. To test this hypothesis, the fiber sizes ranging from 270 nm to 1.04 μm were investigated by changing the concentration of TSPU from 8% to 14% during electrospinning (FIGS. 4A and 4B). Histological examination of devices with different fiber sizes implanted in I.P. space for four weeks revealed that devices with fiber size below ˜500 nm had no cell penetration and induced only thin (<10 μm) cellular overgrowth, whereas those above 500 nm allowed cell penetration (FIG. 4C). Then, devices made from a different material (nylon) were tested for comparison. With a similar fiber size (˜250 nm), the nylon devices also prevented cell penetration after four weeks of I.P. implantation (FIGS. 4A and 4C), but the delamination of different layers in the nanofiber membrane was observed (FIG. 4C). These results confirmed the TSPU nanofibrous device with proper nanofiber sizes was uniquely suitable for cell encapsulation.

### Example 3—Protection of Syngeneic/Allogeneic Cells and Confinement of Proliferative Cells

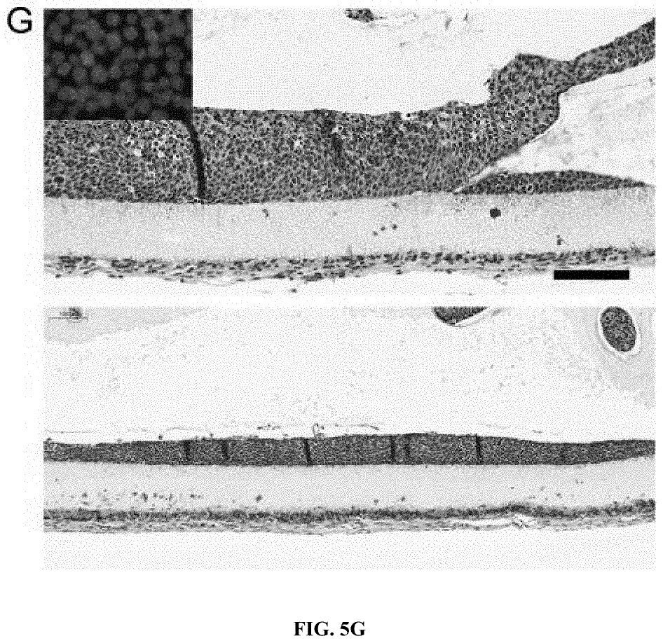

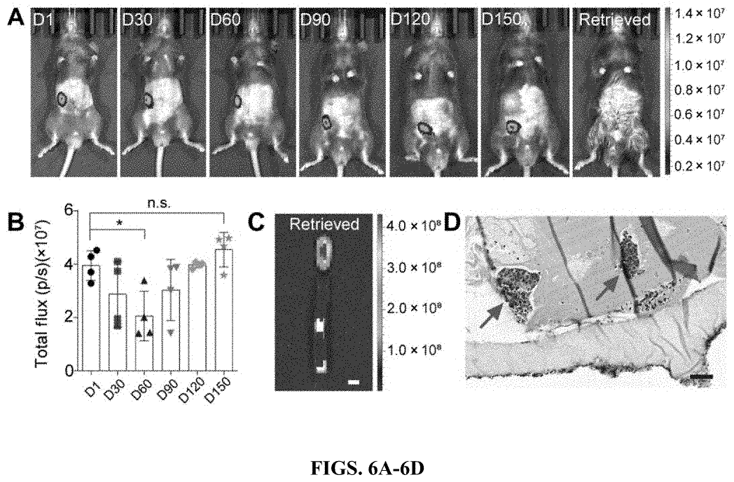

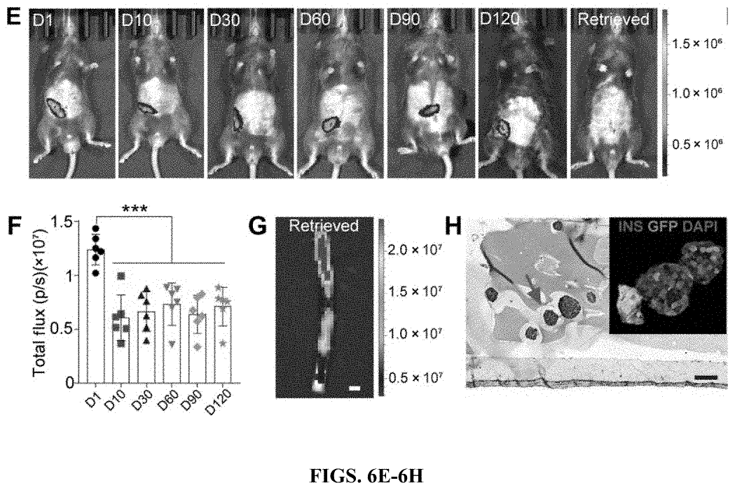

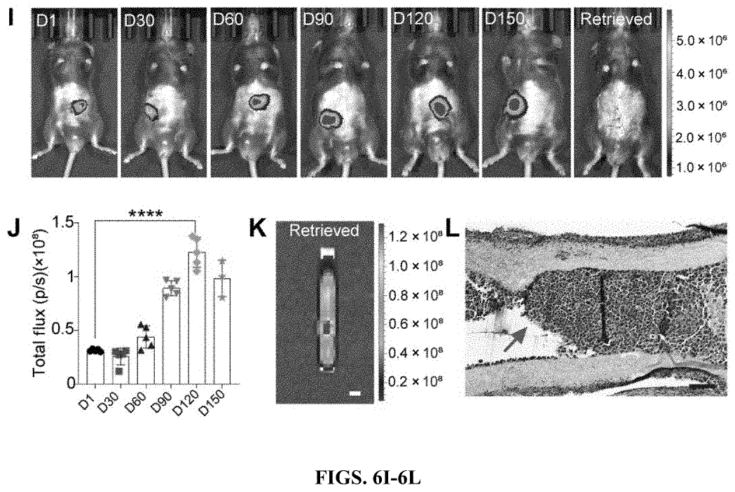

The ability of a cell encapsulation device to prevent cell escape is of paramount importance in SC-β cell delivery due to risks of teratoma formation from undifferentiated cells. However, too tight pore structures will diminish the mass transfer that is necessary for cell viability and function. To test whether the NICE device can provide a solution to the safety/function balance, the inventors encapsulated and implanted luciferase-expressing cells and continuously monitored them over time in C57BL/6 mice. First, syngeneic mesenchymal stem cell (MSC) aggregates (˜100-150 μm in diameter) with green fluorescent protein (GFP)/luciferase expression (FIG. 5A) were encapsulated in NICE devices and implanted in the I.P. space. The bioluminescent signals were localized to the implantation site during the duration (5 months) of implantation and remained steady after an initial drop (FIGS. 6A and 6B). No signal was detected in the recipient animals after device retrieval (FIG. 6A) whereas the retrieved devices were bioluminescent (FIG. 6C), suggesting that the cells were confined and viable in the NICE device for 5 months. To confirm these imaging results, histological analysis was performed on retrieved samples. H&E images showed the confinement and viability of the MSC aggregates within the device (FIG. 6D).

Next, allogeneic GFP/luciferase islets with a size of about 100-150 μm (FIG. 5B) were monitored within the device over time. It was found that 48.9% radiance was detected on day 10 compared to day 1 (FIG. 5E), similar to the initial signal drop in the MSC case. The acute loss of encapsulated cells following implantation was considered common and most likely caused by hypoxia (Carlsson et al., “Markedly Decreased Oxygen Tension in Transplanted Rat Pancreatic Islets Irrespective of the Implantation Site,” Diabetes 50:489-495 (2001) and Komatsu et al., “Impact of Oxygen on Pancreatic Islet Survival,” Pancreas 47:533-543 (2018), which are hereby incorporated by reference in their entirety). However, there was no significant difference (P=0.7798) among day 10, 30, 60, 90 and 120, which demonstrated that the islets remained stable after 10 days (FIGS. 6E and 6F). Post-retrieval imaging of the recipients and devices (FIGS. 6E and 6G) as well as H&E and insulin/glucagon staining confirmed the robust function of the islets after 120 days (FIG. 6H and FIG. 5).

To challenge the device, whether the device could protect invasive cells with a metastatic phenotype and prevent them from escaping was investigate. Allogeneic cancer cell spheroids (4T1) with GFP/luciferase expression (FIG. 5C) were encapsulated in the NICE device. In 2 out of the 6 mice, the bioluminescent signal disappeared after 2 weeks, suggesting early failure possibly due to unintentional defects of the device and/or biological variation. However, in the other 4 mice, the signal increased significantly (P<0.0001) after 60 days and lasted for up to 150 days, indicating the cells were protected (i.e. no immune cell penetration) and proliferated in the device (FIGS. 6I and 6J). In all cases, no bioluminescence outside the implantation site or device was observed. The imaging of retrieved device and H&E staining confirmed that the NICE device was able to support cell viability and prevent them from escape (FIGS. 6K and 6L). Another GFP/luciferase-expressing, allogeneic cell line, NIT-1 β cell spheroids, were also confined and protected by the NICE device for 2 months (FIG. 5G). Taken together, these data showed that the NICE device was cell protective and safe.

### Example 4—Diabetes Correction in Mice with Diverse Rodent Islet Sources

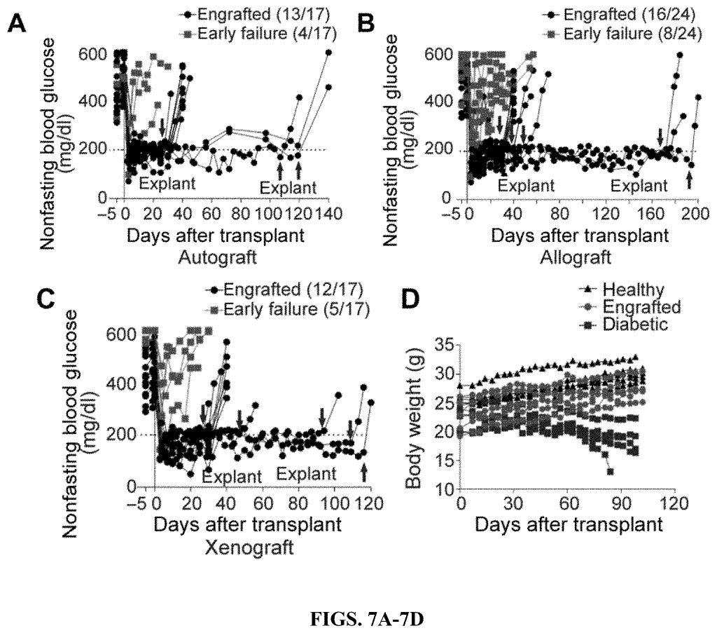

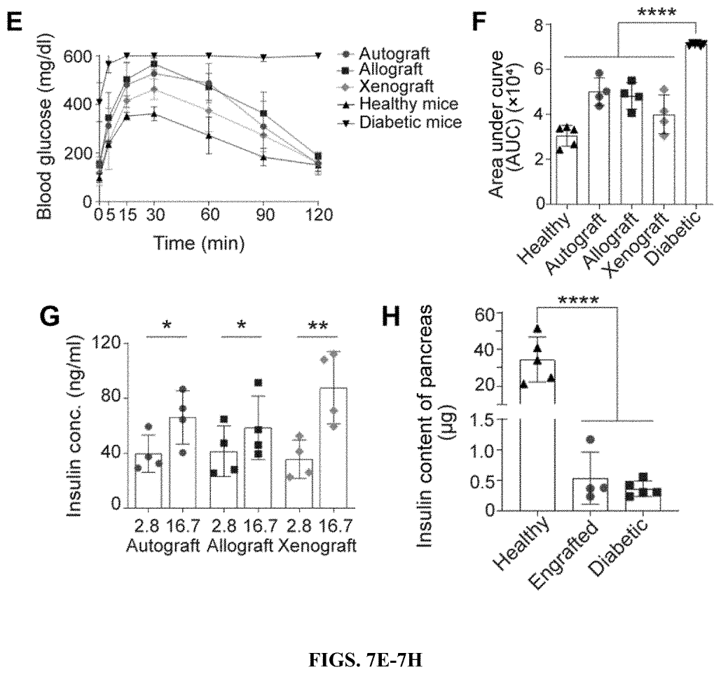

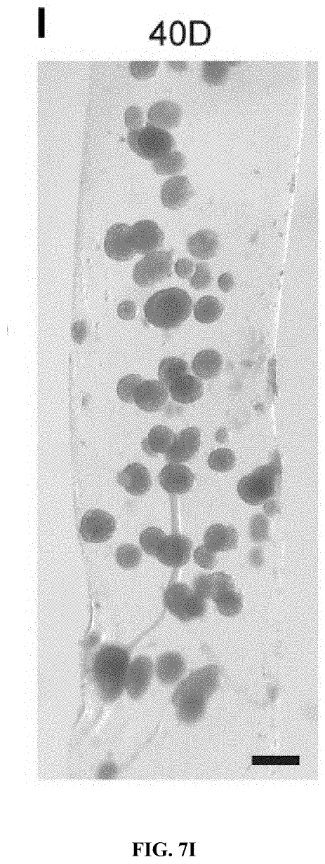

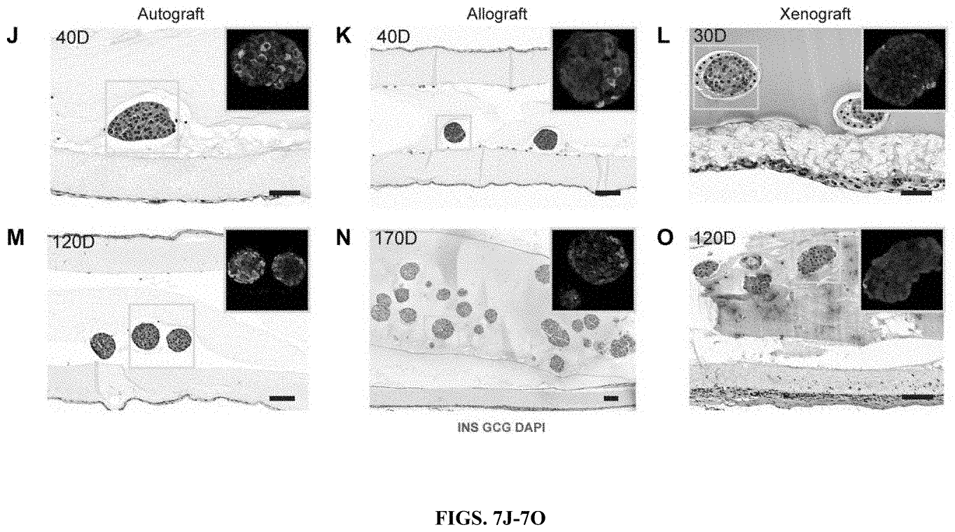

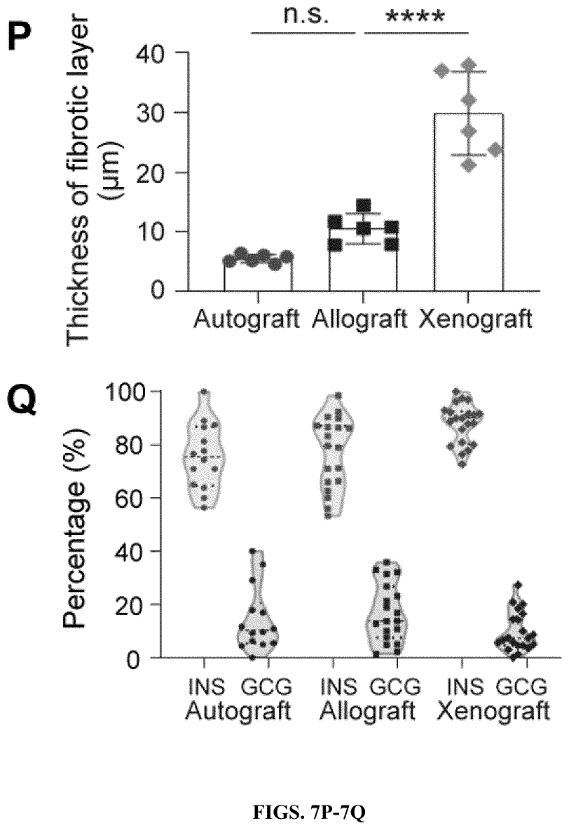

To investigate the function of the NICE device in reversing diabetes, different types of rodent islets (syngeneic, allogeneic and xenogeneic) were encapsulated in the device and implanted in diabetic C57BL/6 mice. Rodent islets were dispersed evenly and embedded in alginate in the NICE device before transplantation. With 600-700 islet equivalent (IEQ) syngeneic islets, the NICE device corrected diabetes in 13 out of 17 mice. The engrafted mice maintained blood glucose within a normal range until device retrieval for as long as 120 days (FIG. 7A). The device encapsulating allogeneic BALB/c islets (600-700 IEQ) engrafted in 16 out of 24 mice, with function for up to 200 days (FIG. 7B). Increasing immunological incompatibility of the transplanted cells by using rat islets (600-700 IEQ) demonstrated function for longer than 100 days (FIG. 7C). In all engrafted animals, the blood glucose concentration dropped within a week of transplantation and increased after graft retrieval at different time points (FIGS. 7A to 7C), confirming that the glucose concentrations were controlled by the grafts. The devices were easily retrievable and there was no tissue adhesion in most cases. The increased body weight of all engrafted mice, similar to healthy mice, showed metabolic recovery, whereas that of diabetic mice dropped continuously (FIG. 7D).

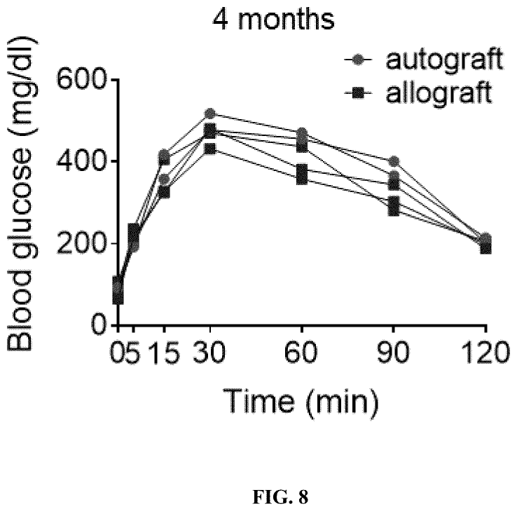

To evaluate the metabolic control of blood glucose, an intraperitoneal glucose tolerance test (IPGTT) was performed on day 30. The IPGTT measurements indicated that all engrafted mice cleared glucose 2 hours post bolus, similar to healthy mice, while the peak value of blood glucose post stimulation in engrafted mice was higher than that of healthy mice. The rate of glucose clearance in all the engrafted mice was significantly (P<0.05) faster than that of the diabetic group (FIG. 7E). Quantitatively, the area under curve (AUC) for glucose clearance in the engrafted mice receiving syngeneic, allogeneic and xenogeneic islets were similar to the healthy mice (FIG. 7F). Notably, there was no difference (P=0.1516) in the AUC among the engrafted mice implanted with different sources of islets. The IPGTT carried out on the engrafted mice with grafts lasting over 4 months also indicated long-term function (FIG. 8). To provide additional proof that the transplanted islets were responsible for glucose control, an ex vivo GSIS test was carried out (FIG. 7G). The responsiveness of islets inside the device indicated their viability and normal function. Lastly, an approximately 60× decrease of insulin content in the pancreas of engrafted mice compared to healthy mice confirmed again that the STZ induction had successfully depleted the β cells in pancreas and that the transplanted grafts were responsible for the observed metabolic control (FIG. 7H).

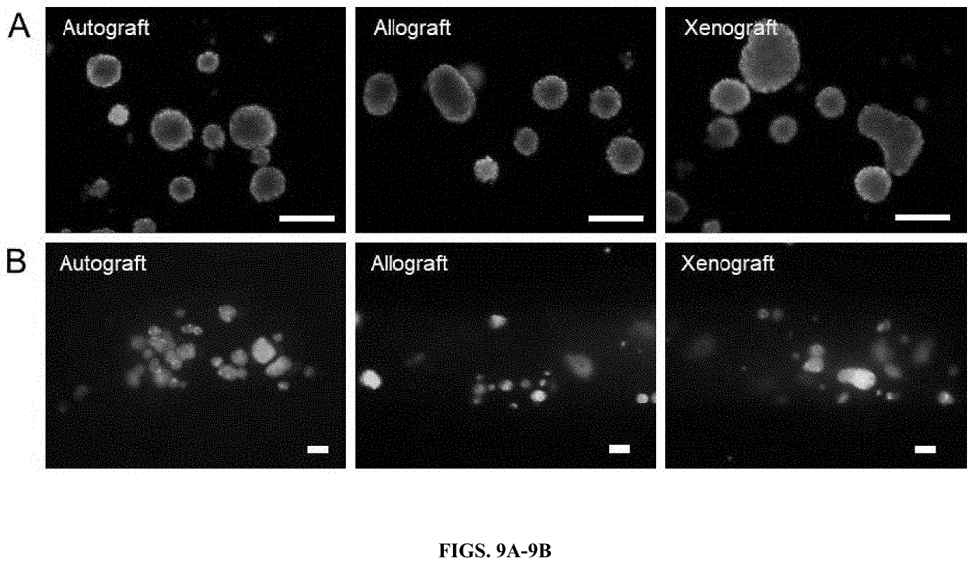

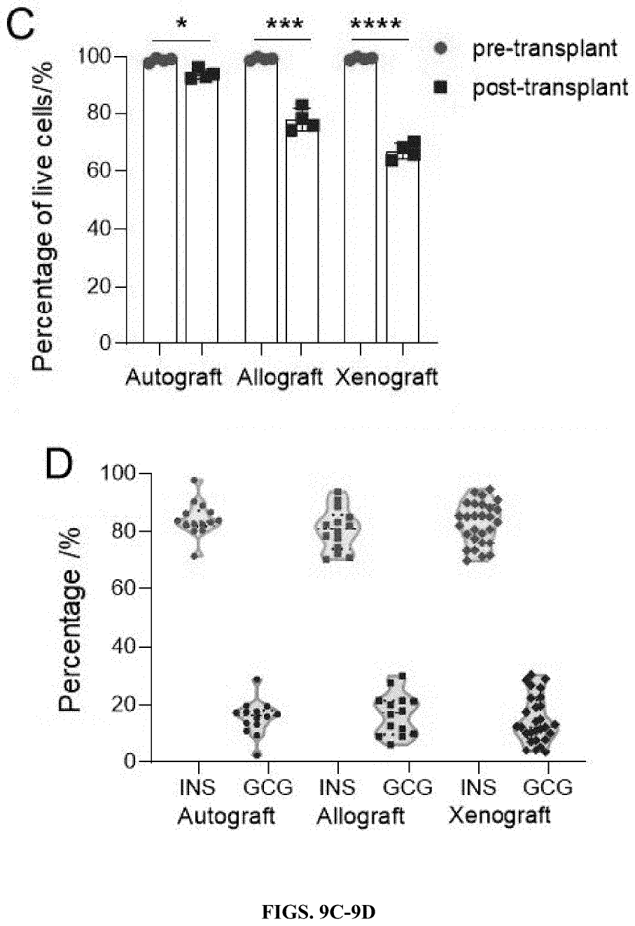

Allogeneic islets in devices retrieved after 40 days appeared healthy, similar to those before transplantation (FIG. 7I). The histological studies showed no tissue adhesion around the device and no cell penetration into the device (FIGS. 7J to 7O). Quantitative analysis of the cellular overgrowth in H&E staining images showed that while there was a relatively thicker fibrotic layer (˜ 30 μm) for xenografts, the fibrotic deposition was minimal (about 10 μm in thickness) for autografts and allografts (FIG. 7P). Islets from different sources encapsulated within the device were viable in the short term (<40 days) (FIGS. 7J to 7L and FIGS. 9A to 9C) and in the long term (>100 days) (FIGS. 7M to 7O). Moreover, islets inside the device were morphologically normal with a round shape, even at long time points (170 days) with a high density in an allogeneic environment (FIG. 7N). Immunofluorescent staining for insulin and glucagon demonstrated that cells maintained their individual hormone identities, many of which were insulin-expressing β cells (FIGS. 7J to 7O). Quantitative analysis of the expression of insulin and glucagon after transplantation showed that on average about 80% of the cells within one islet were insulin-expressing β cells and 15% of cells were glucagon-expressing a cells in autografts and allografts (FIG. 7Q), which were similar to the percentages of α and β cells before transplantation (FIG. 9D). About 88% of insulin+ cells and ˜10% of glucagon+ cells were observed in rat islets after transplantation, also similar to those before transplantation (FIG. 7Q and FIG. 9D). Together these data demonstrated that the NICE device was biocompatible and long-term functional in correcting diabetes in mice with diverse rodent islets.

### Example 5—Immunoprotective Function of NICE Device in Autografts, Allografts and Xenografts

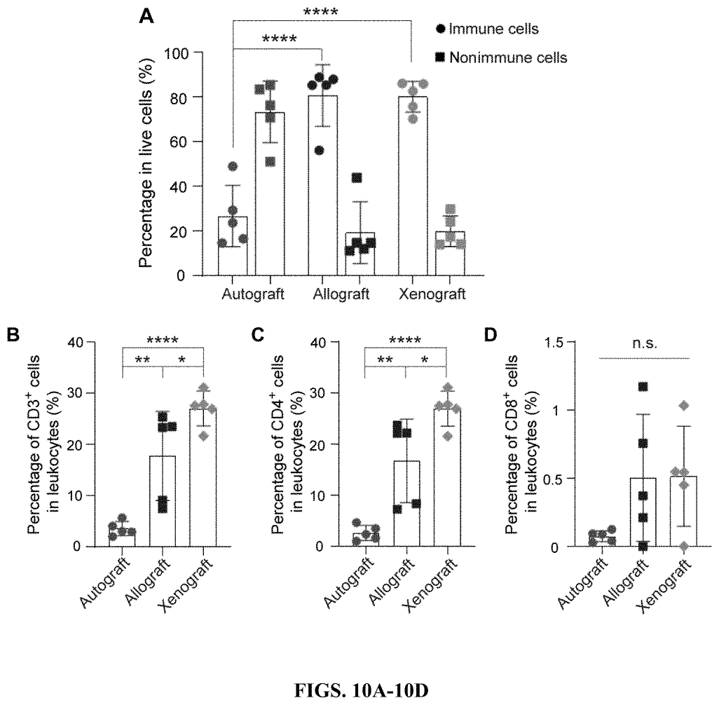

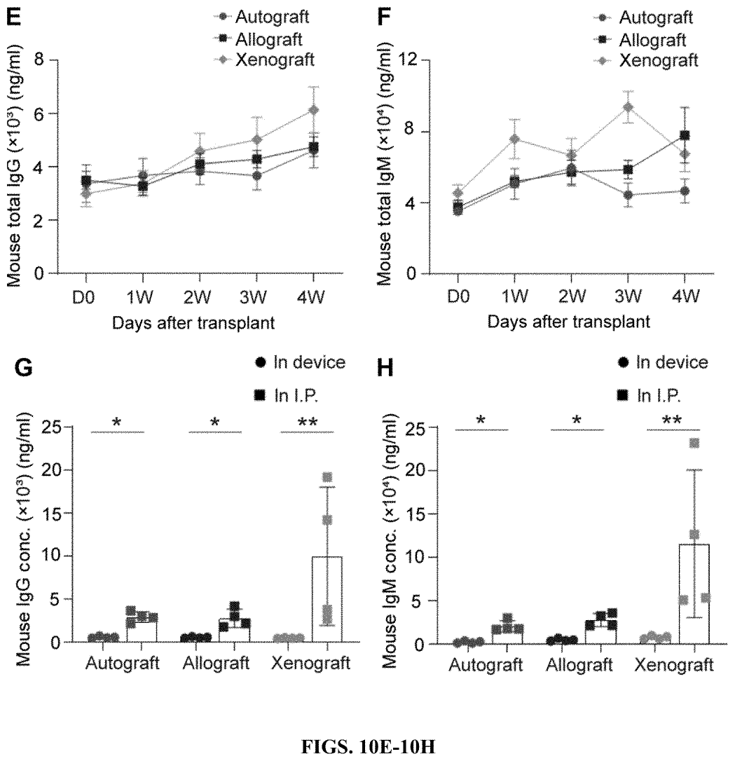

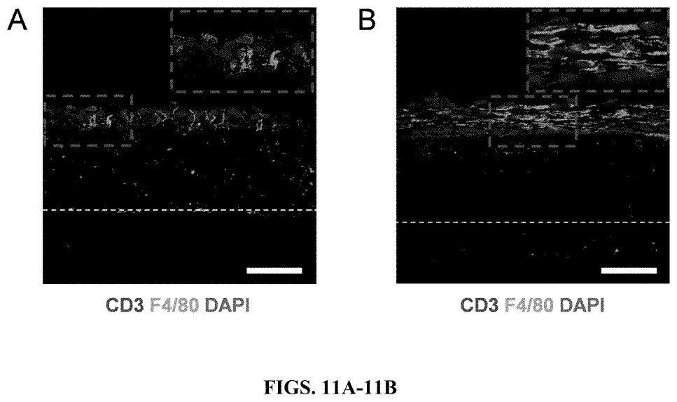

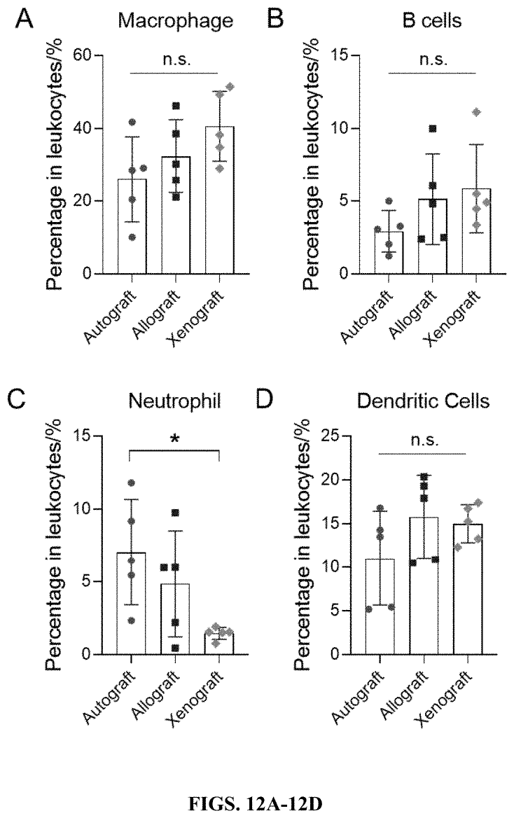

To analyze the immune responses and understand the immunoprotective function of the device under different transplantation scenarios (i.e., autografts, allografts and xenografts), devices were retrieved after one month of implantation and analyzed for the cellular composition surrounding the device and the antibodies within the device. Flow cytometry analysis of the cells deposited on the device showed that 25% of the cells around the encapsulated device were immune cells in autograft group (FIG. 10A). In contrast, 80% of the cells deposited on the device were immune cells in allografts and xenografts, most of which were CD3+ or CD4+ T cells and macrophages (FIGS. 10A to 10C, FIGS. 11 and 12A). Less than 1.5% of cells were CD8+ T cells, which suggested that CD8+ T cells were not recruited to the graft (FIG. 10D). Other types of immune cells found in the fibrotic layer included B cells (about 5%), neutrophils (less than 5%) and dendritic cells (about 15%) (FIGS. 12B to 12D). These data showed that, despite the presence of various types of immune cells, there were very few CD8+ T cells around the device even in xenografts.

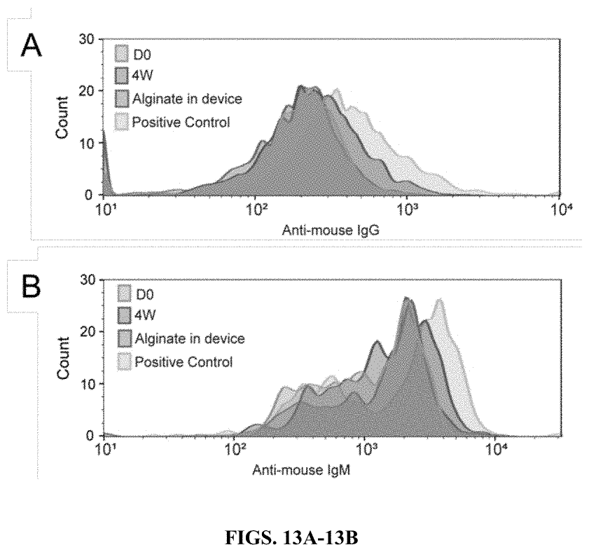

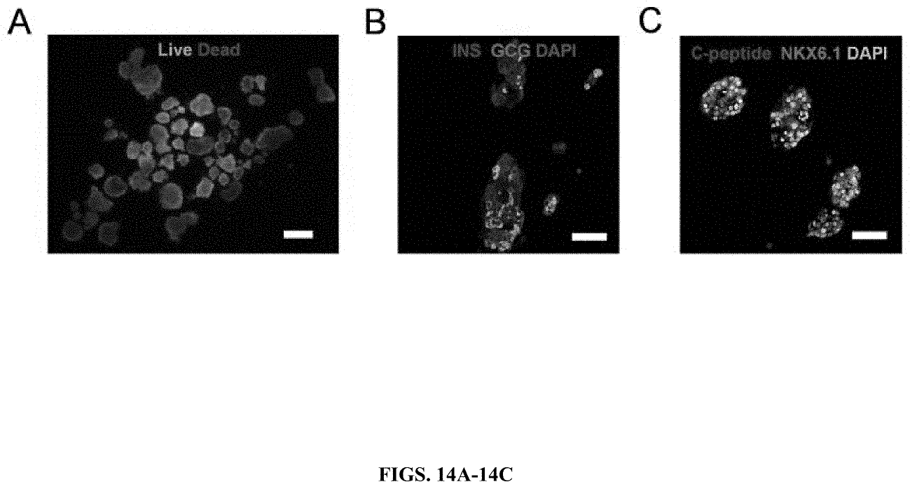

To study the humoral immune response to the encapsulated cells, mouse total antibodies (IgG and IgM) were analyzed in serum at different time points post-transplantation, in I.P. fluid, and inside the device after retrieval. Mouse IgG antibody in xenografts increased significantly (P<0.05) over the 4-week period, while it changed slightly in autografts and allografts (FIG. 10E). Elevated IgM antibodies were observed in the mouse serum in xenografts as early as 7 days after transplantation, with slight decrease at 2 weeks and another peak at 3 weeks (FIG. 10F). Different from xenografts, IgM increased over time in allografts (FIG. 10F). Moreover, it appeared that the antibody response slightly increased in the mice with devices encapsulating xenogeneic islets relative to those with devices encapsulating syngeneic or allogeneic islets (FIGS. 10E and 10F). This response could be elicited by antigens shed from inside the device. The mouse total antibody (IgG and IgM) concentrations in the devices were significantly (P<0.05) lower than those in I.P. fluids in all grafts including xenografts (FIGS. 10G and 10H). To determine whether the IgG and IgM detected in allografts were donor specific alloantibodies, BALB/c T cells were incubated with serum withdrawn from the recipients with allografts and labeled with FITC anti-mouse IgG or anti-mouse IgM. Serum extracted from sensitized C57BL/6 mice with BALB/c islets transplanted in kidney capsules at 4 weeks post-transplantation was used as positive control. The flow cytometry data showed that there were anti-BALB/c alloantibodies (IgG and IgM) detected in C57BL/6 serum with allografts at 28 days post-transplantation, although with much lower concentration than in the serum of sensitized mice (FIG. 13). However, almost no alloantibodies were present inside the device (FIG. 13). These data confirmed the immunoprotective function of the device.

### Example 6—the NICE Device that Supports the Function of Human Islets in Immunodeficient Mice

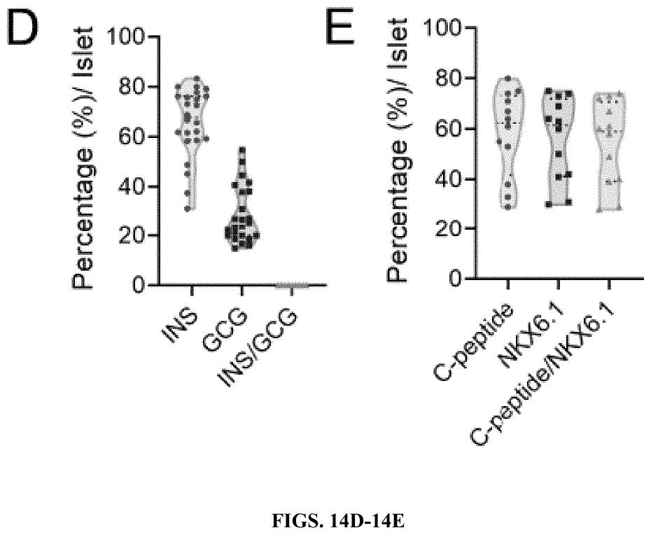

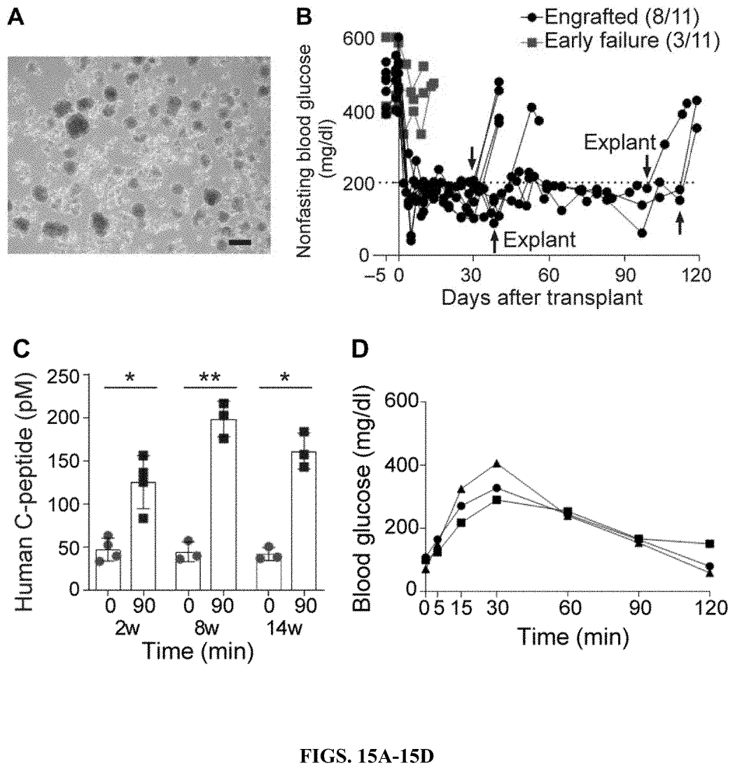

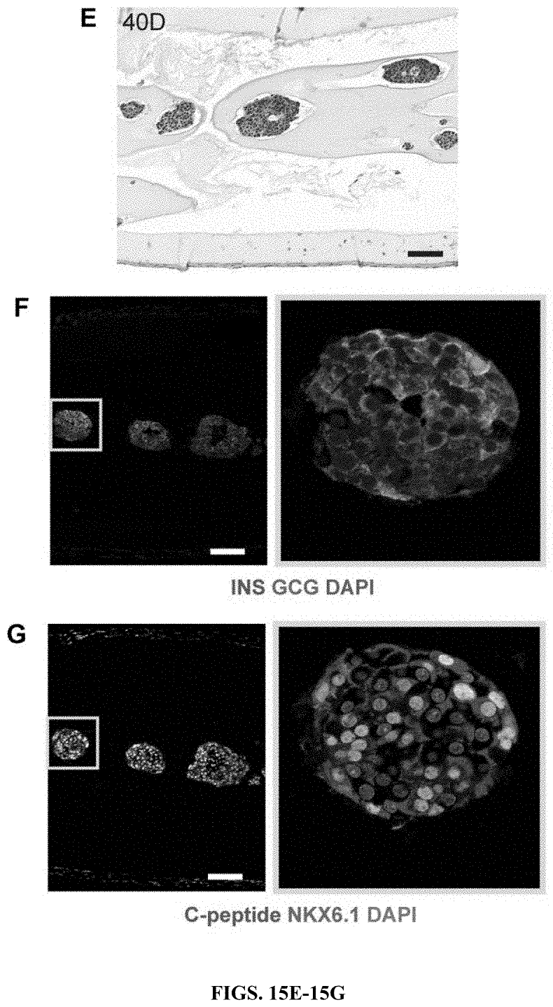

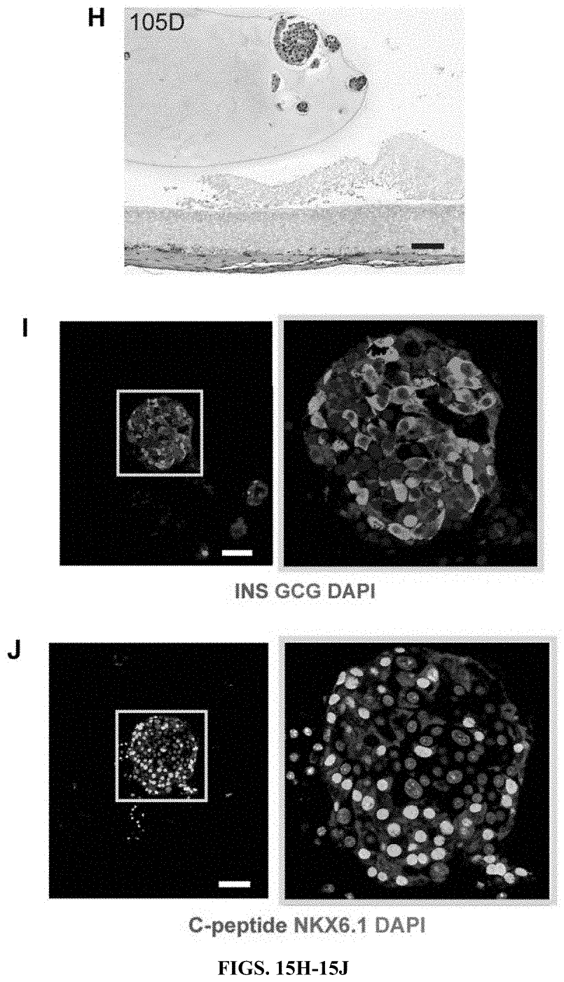

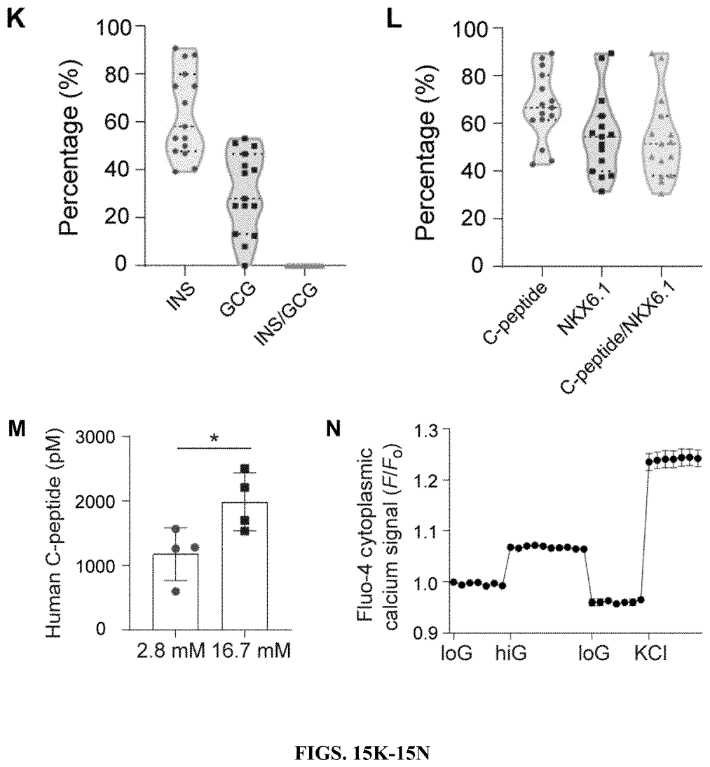

To demonstrate the potential of the NICE device for human islet encapsulation, the device was transplanted into diabetic, immunodeficient SCID-beige mice. Human islets with variable sizes but acceptable viability (70-90%) (FIG. 11A and FIG. 14A) were received, encapsulated (˜1700 IEQ per mouse) and transplanted. From immunofluorescent staining studies, before transplantation the human islets expressed D cell markers such as insulin, C-peptide and transcription factor NKX6.1 (FIGS. 14B and 14C). Eight out of eleven recipient mice recovered from diabetes immediately (within a week) and maintained normoglycemia for up to 100 days before device retrieval (FIG. 15). Human C-peptide after IPGTT at 2-, 8- and 14-week time points was detectable in the serum and the concentration was significantly (P<0.05) increased at 90 mins compared to 0 min (FIG. 15C), suggesting that human islets inside the device were responsive to glucose and secreted insulin. Blood glucose measurements during an IPGTT at week 8, representative of other time points, showed that the engrafted mice cleared glucose within 2 hours (FIG. 15D). The retrieved device had minimal cellular overgrowth and healthy islets were observed in the device in both the short term (FIGS. 15E to 15G) and long term (FIGS. 15H to 15J). Immunofluorescent staining showed insulin and glucagon expression (FIGS. 15F and 15I) and C-peptide and NKX6.1 expression (FIGS. 15G and 15J) at 40 days and 105 days, although some small necrotic areas were present in larger islets. Quantitative analysis of the expression of insulin and glucagon after transplantation showed that about 64% of cells within one islet were insulin-expressing β cells and 31% of cells were glucagon-expressing a cells (FIG. 15K), which were similar to the percentages of α and β cells of human islets before transplantation (FIG. 14D). Also, no polyhormonal cells were observed either before or after transplantation. Further analysis revealed that about 67% of cells within one islet were C-peptide+ cells, 55% of cells were NKX6.1+ and 54% were C-peptide+/NKX6.1+ β cells (FIG. 15L), which were also similar to those before transplantation (FIG. 14E). To further verify that the transplanted islets were responsible for glucose control, the engrafted devices were retrieved one month after implantation and an ex vivo GSIS test was carried out. The retrieved devices secreted significantly (P<0.05) more human C-peptide in high glucose solution than that in low glucose solution (FIG. 15M). In addition, dynamic calcium imaging was conducted on extracted, dissociated single cells. The cells responded with increased calcium influx when exposed to high glucose solution or potassium chloride (KCl), an agent to depolarize the cell membrane (FIG. 15N), confirming their functions.

### Example 7—the NICE Device that Supports the Function of Human SC-β Cells in Immunodeficient Mice

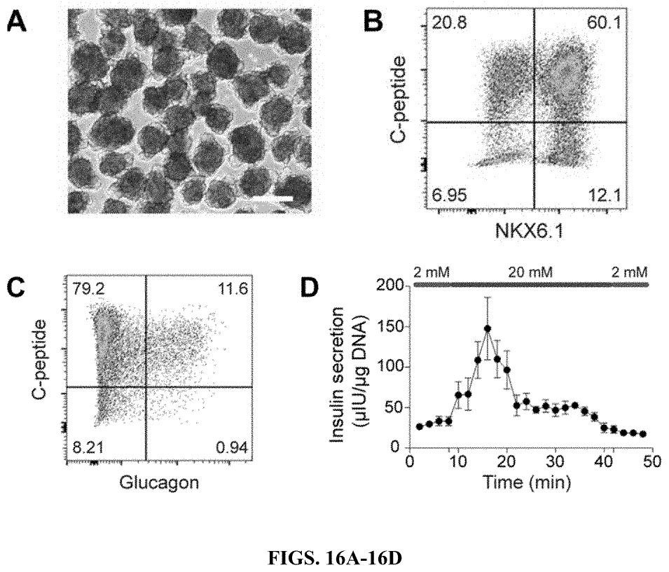

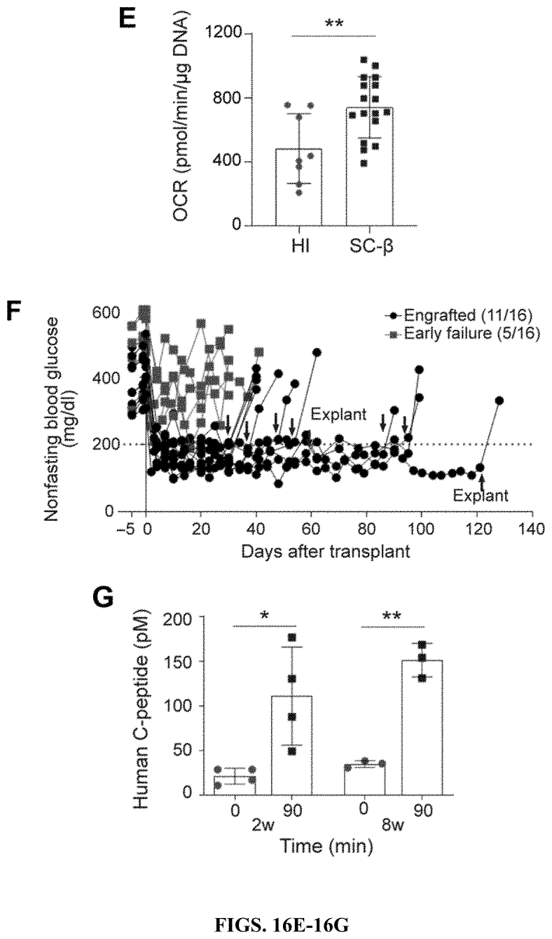

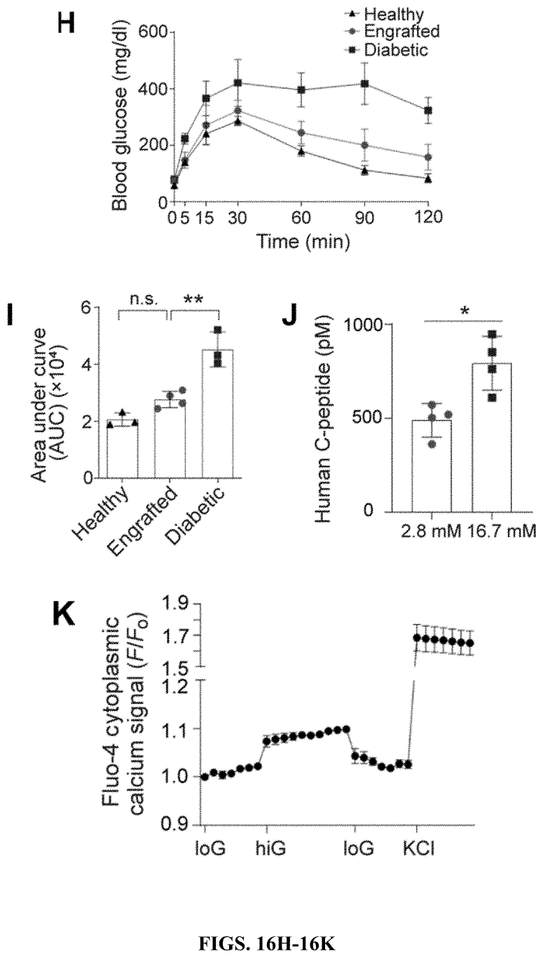

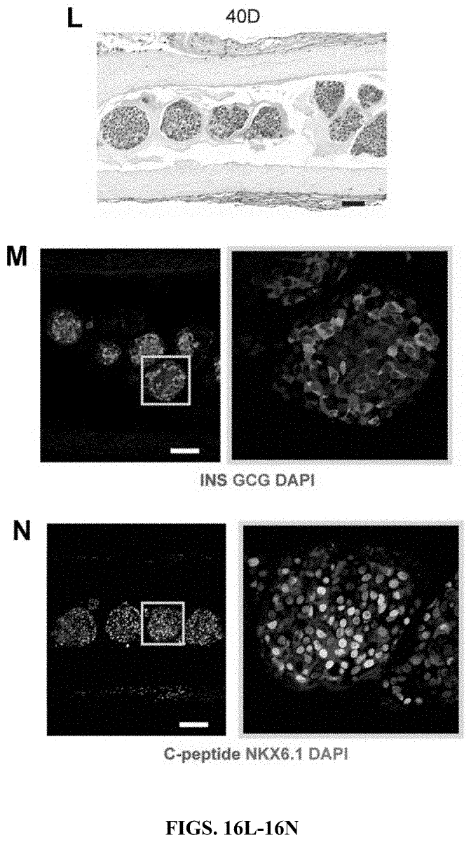

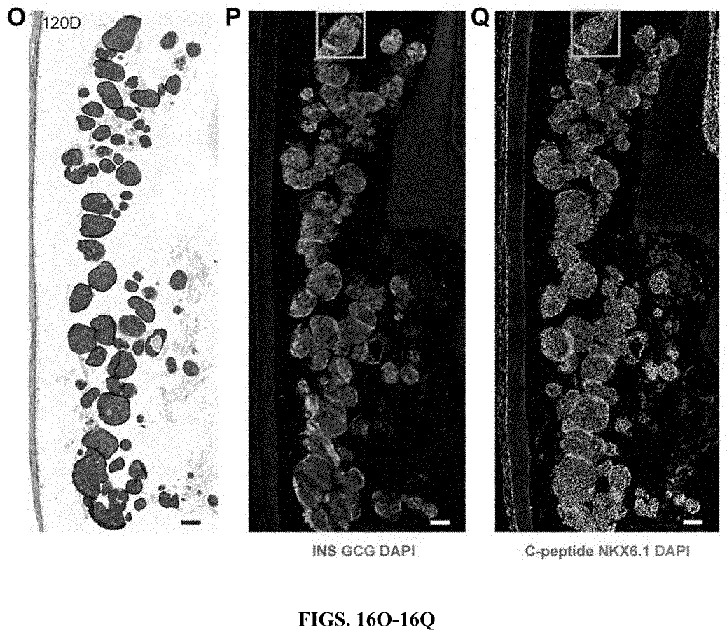

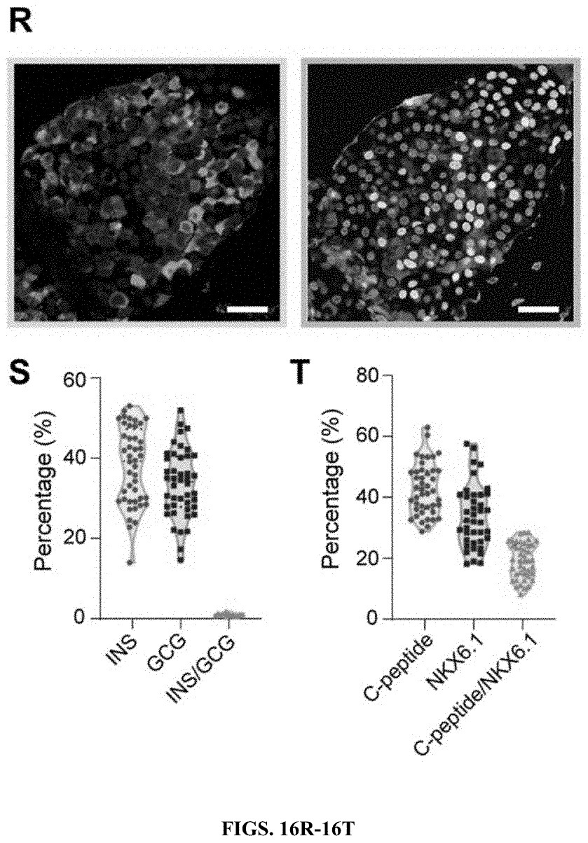

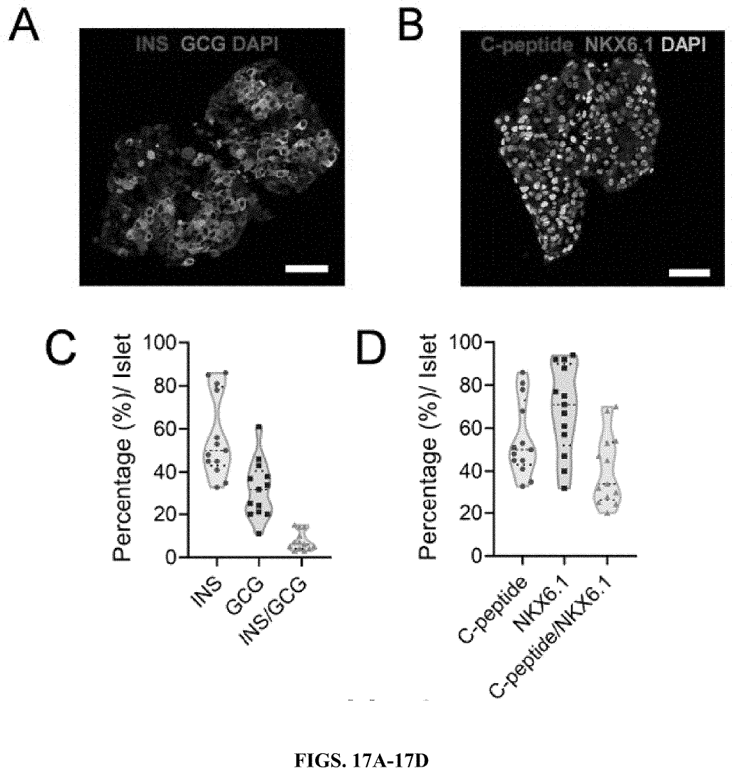

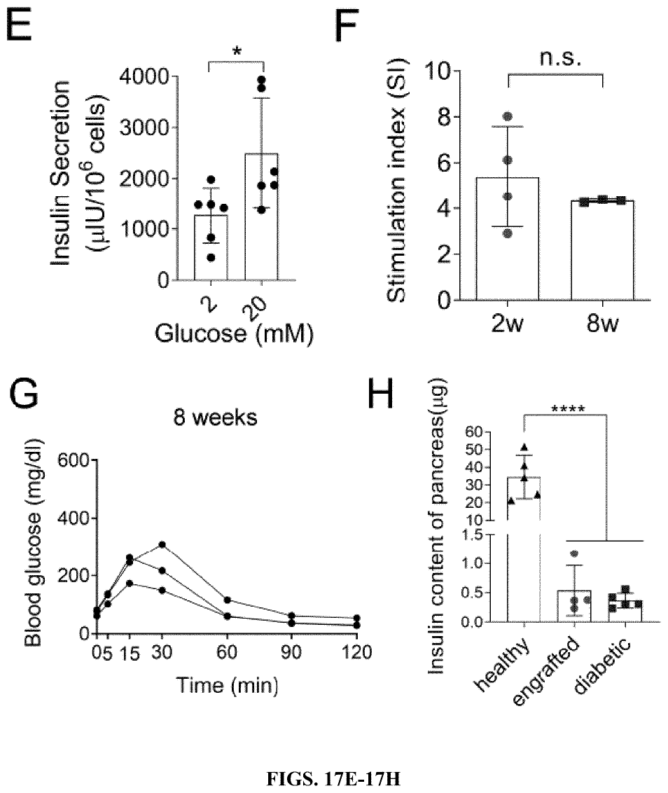

From a broad point of view, the highest impact of a cell encapsulation device that is both safe and functional would be its use for delivery of human SC-β cells. To provide evidence that the NICE device has this potential, human SC-β cells were encapsulated (Pagliuca et al., “Generation of Functional Human Pancreatic β Cells In Vitro,” Cell 159:428-439 (2014), and Velazco-Cruz et al., “Acquisition of Dynamic Function in Human Stem Cell-Derived β Cells,” Stem Cell Rep Stem Cell Rep 12:012 (2019), which are hereby incorporated by reference in their entirety) and implanted in diabetic immunodeficient NSG mice (˜2500 clusters per mouse). The SC-β cells had a more uniform size (˜100-150 μm in diameter) compared to the human islets (FIG. 16A). From immunofluorescent staining studies, before transplantation the SC-β cells expressed β cell markers such as insulin, C-peptide and NKX6.1 (FIGS. 17A and 17B). About 60% of cells expressed both C-peptide and NKX6.1 as measured by flow cytometry (FIG. 12B) and 12% were polyhormonal cells with expression of both C-peptide and glucagon (FIG. 12C). Slightly lower C-peptide expression (˜55%) was obtained by quantifying the immunofluorescent staining images (FIGS. 17C and 17D). In a dynamic GSIS assay, SC-β cells secreted higher amounts of insulin with a clear first- and second-phase response when exposed to increased glucose (FIG. 16D). Static GSIS also demonstrated that before transplantation SC-β cells can secrete more insulin in high glucose solution than that in low glucose solution (FIG. 17E). Furthermore, oxygen consumption rate (OCR) measurement showed that SC-β cells had a higher OCR normalized to DNA than human islets, indicating SC-β cells might have a higher metabolism than human islets (FIG. 16E).

The device with SC-β cells corrected blood glucose immediately (within a week) in 11 out of 16 mice, which was maintained for up to 120 days (FIG. 16F). Recipients became diabetic again after explanting the devices, showing that blood glucose was controlled by the NICE device with SC-β cells (FIG. 16F). Human C-peptide in the blood at 90 minutes post glucose challenge (111±21.3 pM) was significantly (P<0.05) higher than the fasting value (21.4±7.7 pM) 2 weeks after implantation (FIG. 16G). At 8 weeks, there was also glucose responsive C-peptide secretion at 90 min (151.2±15.4 pM) compared to that at 0 min (34.8±3.1 pM) (FIG. 16G). There was no significant difference (P=0.4511) in terms of the stimulation index at 2 weeks (5.4±1.9) and that at 8 weeks (4.3±0.1), suggesting stable device function during that period (FIG. 17F). For the engrafted mice studied at 30 days, glucose clearance after IPGTT was completed by 120 minutes (FIG. 16H); the AUC of the glucose concentration curve was similar to that of healthy mice, while significantly (P<0.01) less than that of diabetic mice (FIGS. 16H and 16I). An IPGTT study carried out at 8 weeks also demonstrated the long-term function of SC-β cells in the device (FIG. 17G). In addition, the total insulin content in the pancreas of the engrafted mice was significantly (P<0.0001) lower than that of healthy mice, which again confirmed that the transplanted grafts were responsible for the observed metabolic control (FIG. 17H).

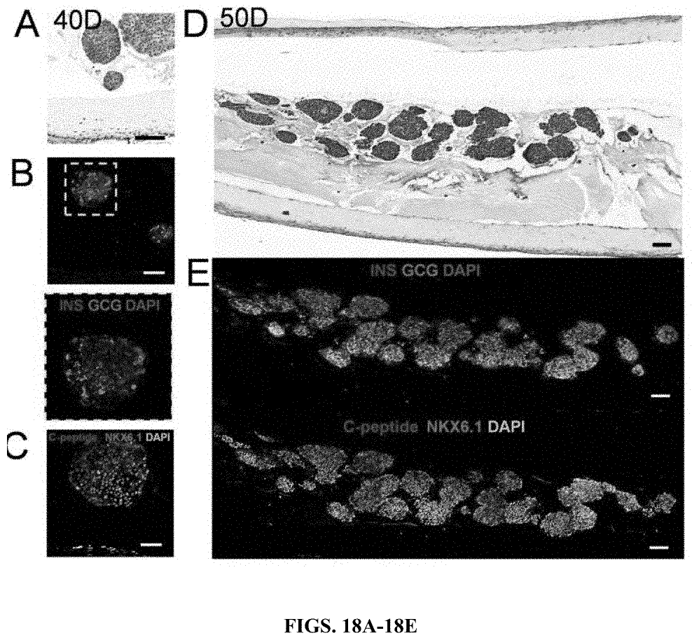

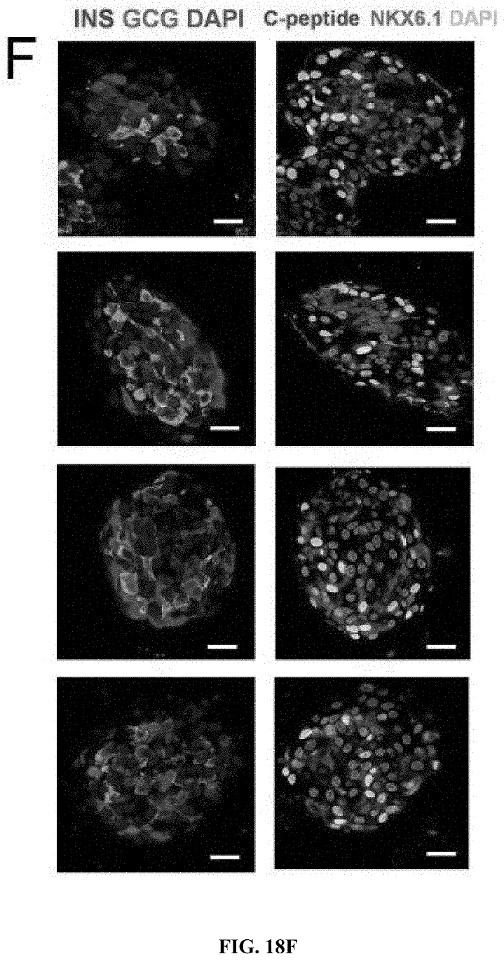

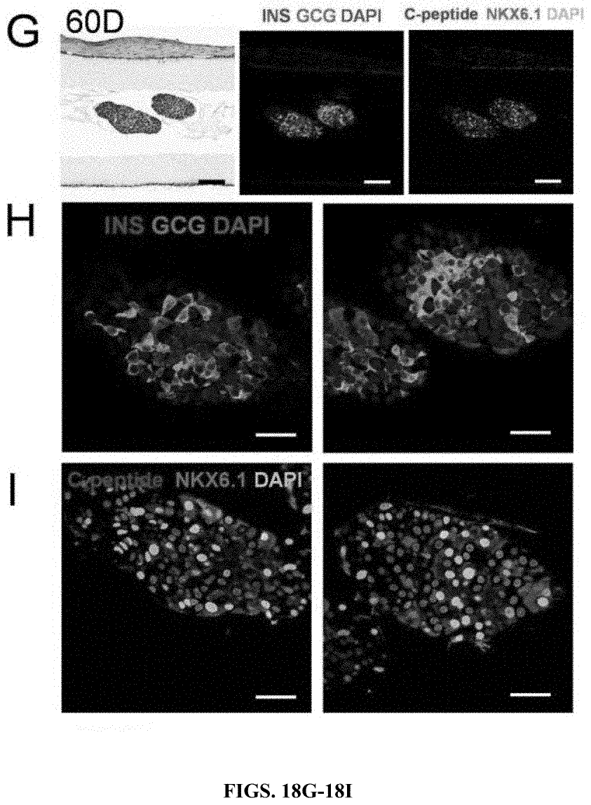

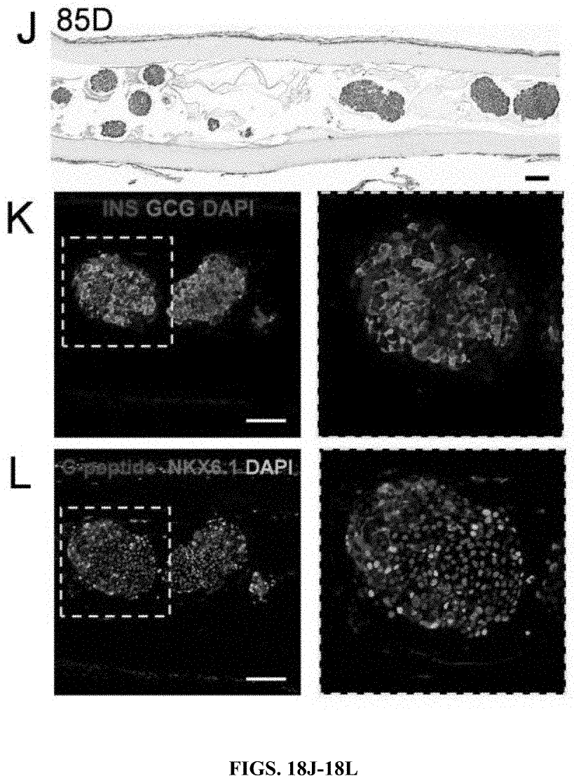

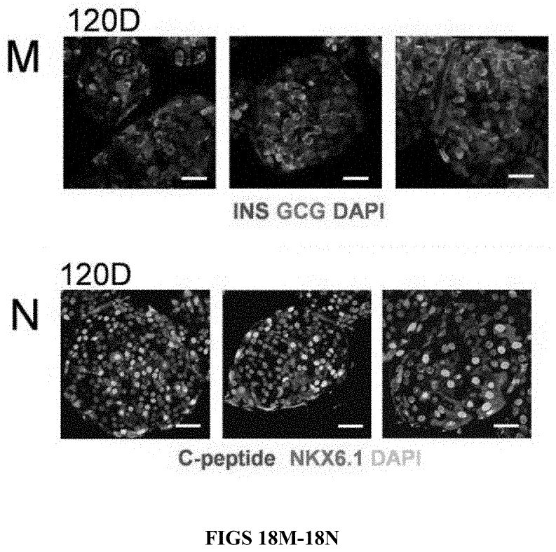

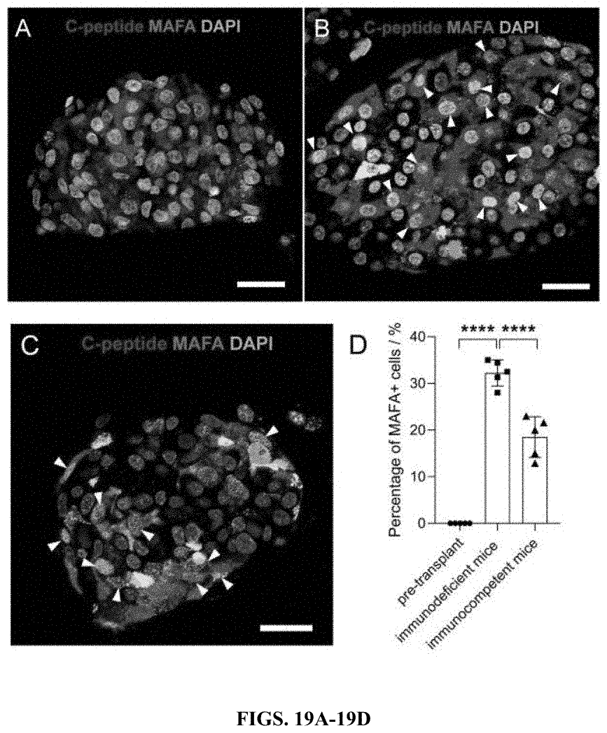

Ex vivo GSIS test showed that devices retrieved at one month secreted significantly (P<0.05) more human C-peptide in high glucose solution than that in low glucose solution (FIG. 16J). Dynamic calcium imaging conducted on dissociated cells corroborated the GSIS results: the retrieved SC-β cells exhibited glucose- and KCl-responsive calcium influx (FIG. 16K). SC-β cells explanted after 40 days and 120 days remained viable with a healthy and round morphology, although the cells were packed at a high density within the device (FIG. 16L). The expression of insulin, glucagon, C-peptide and NKX6.1 of the cells retrieved at different time points (up to 120 days) indicated device function in both short and long terms (FIG. 16 and FIG. 18). Quantitative analysis of the hormone expression within each cell aggregate post transplantation averaged over four recipients showed that about 38% of cells were insulin-expressing β cells and 34% of cells were glucagon-expressing a cells (FIG. 16S). Polyhormonal cells were observed prior to transplantation, but less than 1% of cells expressed both insulin and glucagon after retrieval (FIG. 16S). About 43% of cells within one aggregate expressed C-peptide, 37% of cells expressed NKX6.1 and 19% expressed both markers (FIG. 16T). About 32% of cells within the device expressed transcription factor MAFA, a maturation marker of β cells, after transplantation (FIG. 19). The decline of C-peptide- or insulin-expressing cells after transplantation may be due to the maturation of polyhormonal cells observed before transplant into a cells in vivo (Pagliuca et al., “Generation of Functional Human Pancreatic β Cells In Vitro,” Cell 159:428-439 (2014), and Rezania et al., “Reversal of diabetes with insulin-producing cells derived in vitro from human pluripotent stem cells,” Nat Biotechnol 32:1121 (2014), which are hereby incorporated by reference in its entirety). Despite a lower percentage of β cells, the percentage of a cells in SC-β cells was similar to that in human islets; a cells are important in the crosstalk with β cells and controlling blood glucose concentration (Rodriguez-Diaz et al., “Paracrine Interactions within the Pancreatic Islet Determine the Glycemic Set Point,” Cell Metab 27: 549-558.e4 (2018), and Svendsen et al., “Insulin Secretion Depends on Intra-islet Glucagon Signaling,” Cell Reports 25:1127-1134.e2 (2018), which are hereby incorporated by reference in their entirety). These results show that the NICE device supported the function of the human SC-β cells in immunodeficient mice.

### Example 8—Testing of the NICE Device with Human SC-β Cells in Immunocompetent Models

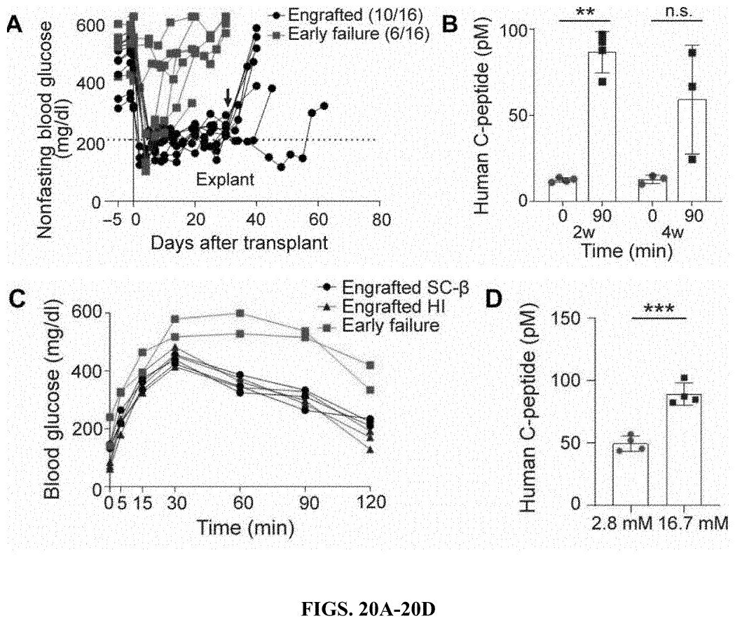

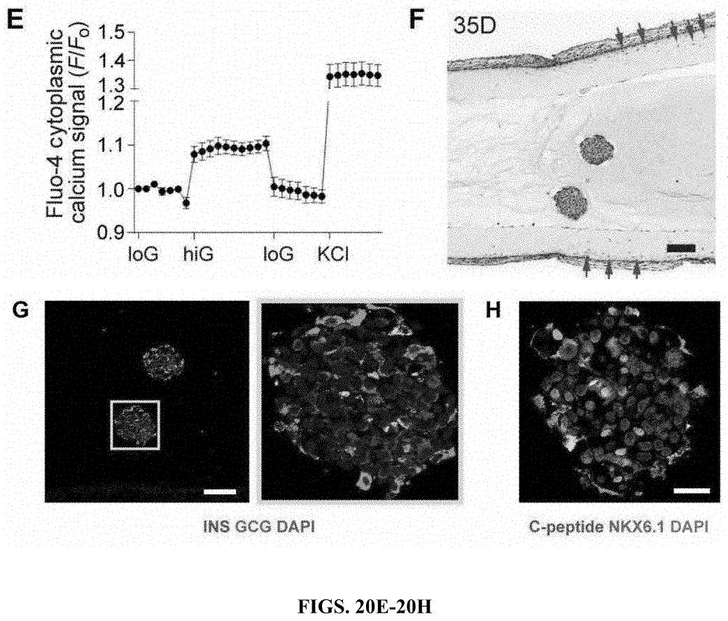

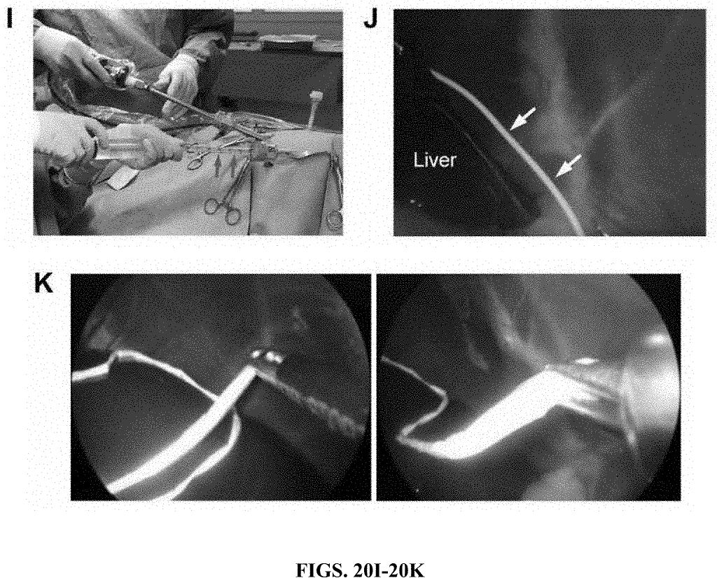

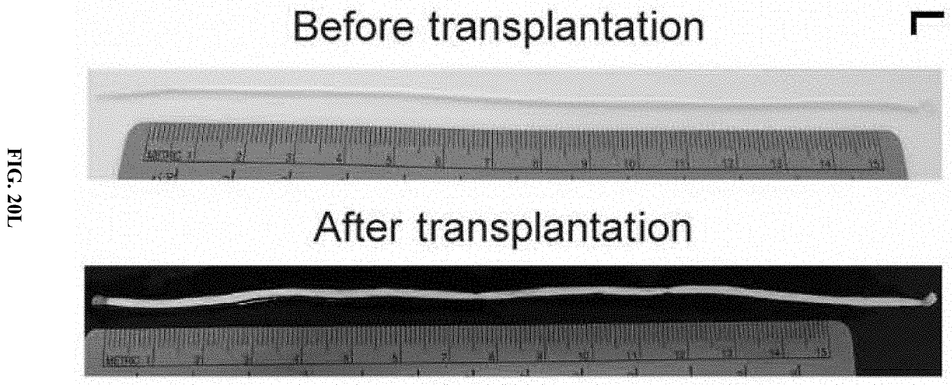

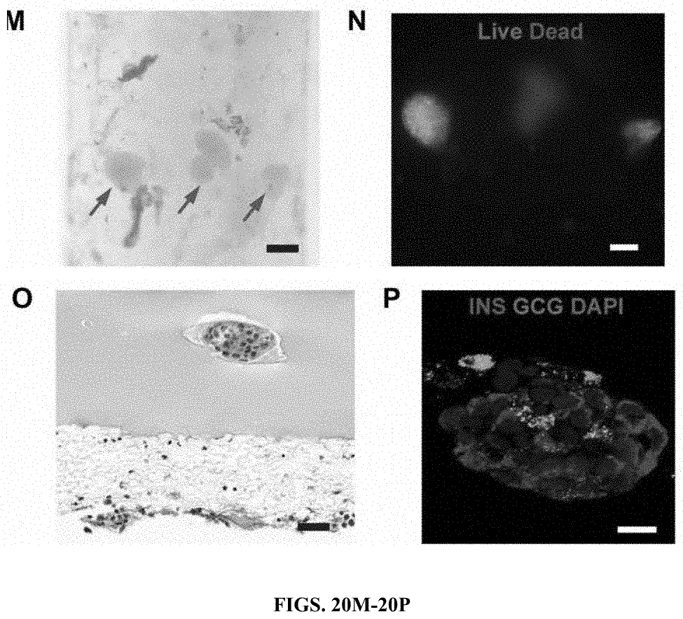

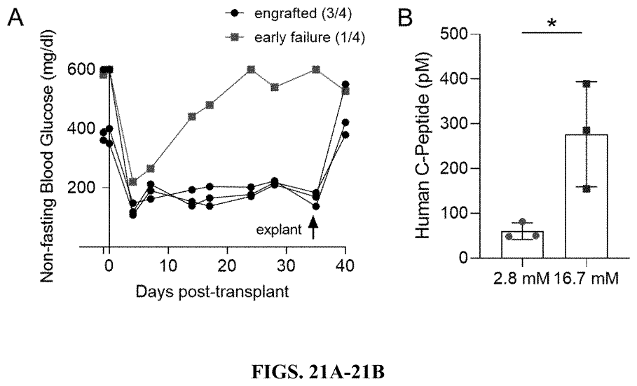

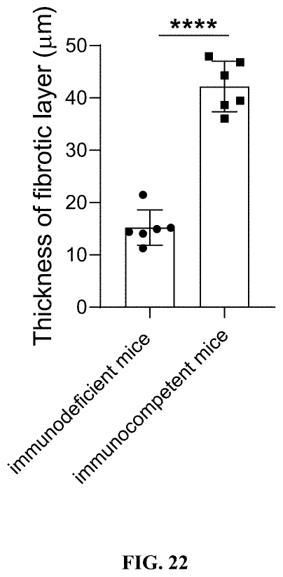

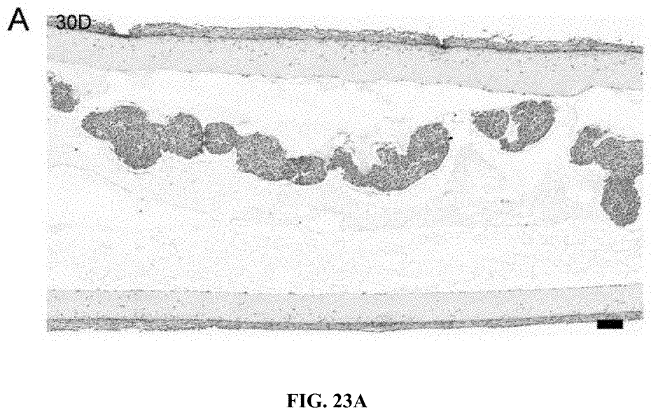

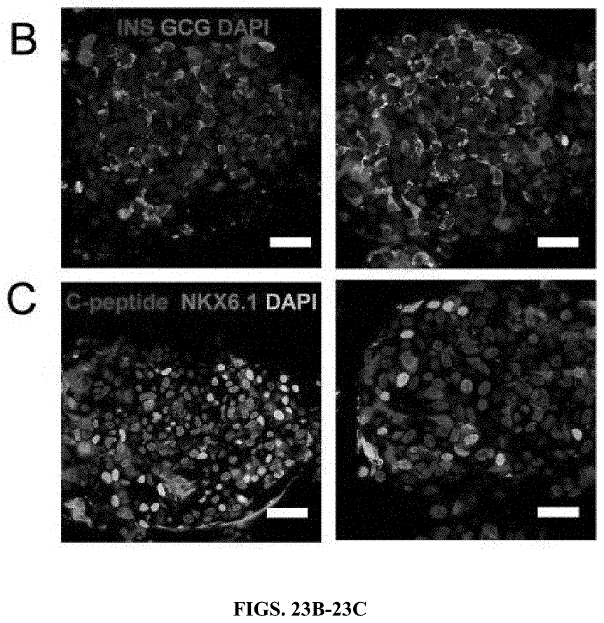

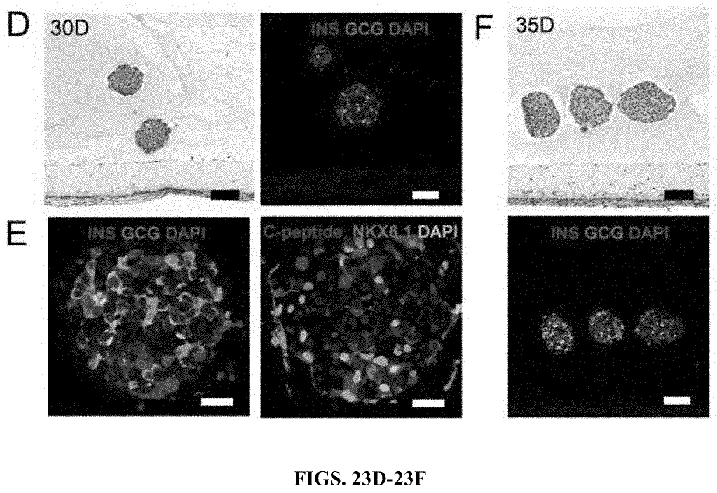

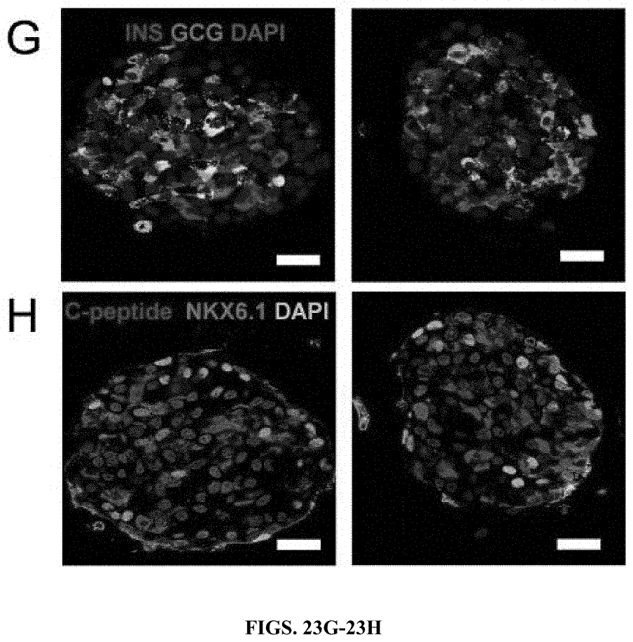

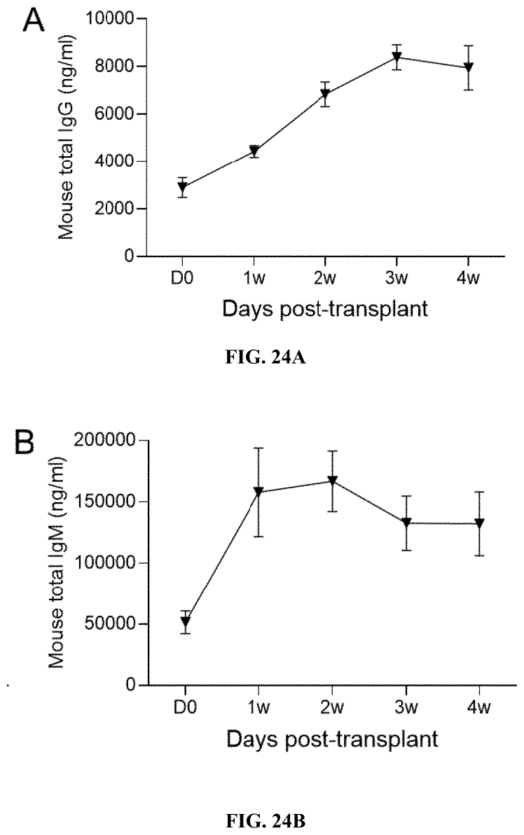

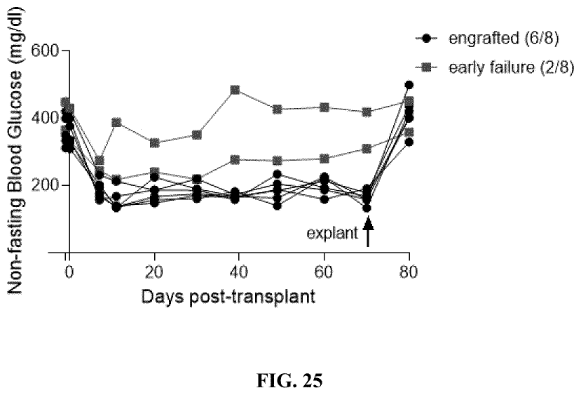

After confirming the function of encapsulated human SC-β cells in immunodeficient mice, the applicants explored the use of the NICE device for delivering cells in immunocompetent diabetic mice and healthy dogs. It is noted that these xenotransplantation models may be more challenging than allogeneic, human SC-β cells-to-human transplantation, and the explorations here are intended to test the robustness, safety, scalability and retrievability in the setting of SC-β cell delivery but not necessarily the full, long-term function of the device. Nevertheless, when devices with the human SC-β cells (˜2500 clusters) were implanted in diabetic C57BL/6 mice, 10 out of 16 mice became normoglycemic immediately (within a week) after transplantation and maintained normoglycemia for up to 8 weeks (FIG. 20A), a result better than anticipated. Human C-peptide in the blood of mice with engrafted SC-β cells at 90 minutes post glucose challenge (86.7±10.4 pM) was significantly (P<0.01) higher than the fasting value (12.4±1.1 pM) 2 weeks after implantation (FIG. 20B). However, at 4 weeks, there was no significant difference (P=0.11) of C-peptide secretion before and after glucose injection, which indicated that the function of cells declined after 4 weeks (FIG. 20B). Blood glucose measurement during IPGTT at 2 weeks showed that the engrafted mice cleared glucose within 2 hours, whereas the mice with early failed grafts remained at high glucose concentrations (FIG. 20C). The mice engrafted with SC-β cells showed blood glucose clearance following IPGTT similar to those engrafted with human islets (FIG. 20C), which were included as a control (FIG. 21A). The SC-β cells in retrieved devices secreted significantly (P<0.001) more human C-peptide in high glucose solution than that in low glucose solution following ex vivo GSIS test (FIG. 20D). In addition, the dynamic calcium imaging conducted on retrieved cells showed glucose-responsive calcium influx (FIG. 20E). Histological studies showed that the SC-β cells were reasonably healthy after 35 days, despite the formation of a more severe FBR than that observed in immunodeficient mice and the presence of some cells having penetrated into the membrane (FIG. 20F and FIG. 22). Immunofluorescent staining also confirmed the expression of insulin, glucagon, C-peptide, NKX6.1 (FIGS. 20G and 20H and FIG. 23) and MAFA. It is noted that human SC-β cells elicited much stronger (P<0.01) immune responses than rodent islets in mice (FIGS. 24A-24B). Applying immunosuppression—such as treatment of rapamycin which suppresses T cell activation and proliferation—led to improved engraftment of encapsulated SC-β cells in immunocompetent mice (FIG. 25).

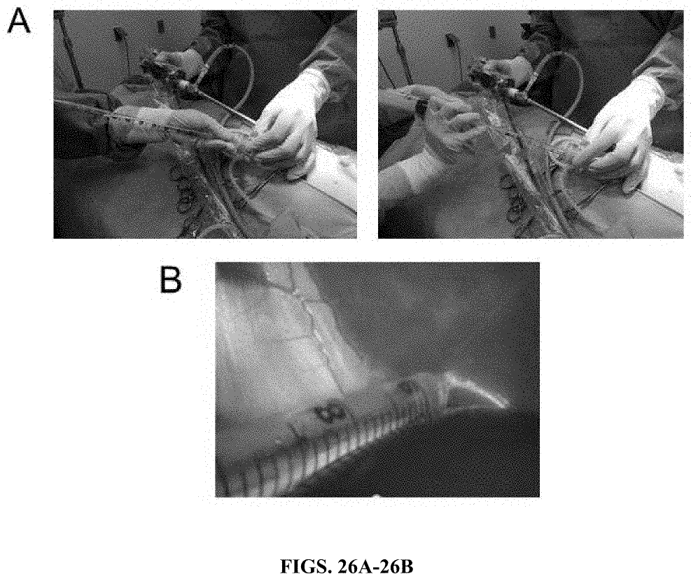

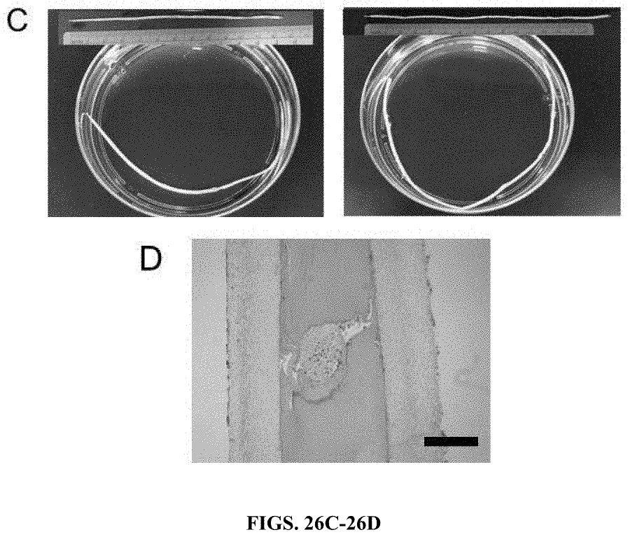

To support the concept that the NICE device is scalable, easy to handle and implant, and retrievable, devices containing human SC-β cells were implanted into healthy dogs (n=3) without administering immunosuppressive drugs. TA sub-therapeutic dose (2500 clusters) of SC-β cells was loaded into 1 mm-diameter, 17 cm-long devices. In a typical procedure, the device was placed in a 10 mL pipette, which was then inserted through a trocar (FIG. 20I and FIG. 26A). Under laparoscopic visualization, the device was implanted into the dog's peritoneal cavity by flushing the device with saline through the trocar (FIG. 26B). The device was implanted freely in the I.P. cavity without anchoring and placed in the cranial abdomen near the liver (FIG. 20J). Two weeks later, the device was retrieved by grasping one end of the device using laparoscopic forceps and pulling the device through a trocar (FIG. 20K). In two dogs, minor adhesions occurred between the peritoneal wall and a short segment of the device. In another dog, there was a point adhesion to the liver. In all 3 dogs, adhesions were considered minor and easy to separate, and the devices were all retrievable in their entirety (FIG. 20L, FIG. 26C). The SC-β cells were still encapsulated in intact alginate hydrogel within retrieved devices (FIG. 20M). In addition, while many cells were dead (FIG. 26D), likely due to the severe immune responses in the challenging xenogeneic setting, it was surprising to observe viable cells through live/dead staining (FIG. 20N). The H&E staining and immunofluorescent staining of insulin and glucagon also confirmed the viability and function of some cells (FIGS. 20, O and P). These stringent, exploratory studies in immunocompetent mice and dogs, which may be more immunologically challenging than the scenario of human trial, demonstrated the feasibility of the NICE device for SC-β cell delivery from a procedural perspective.

### Example 9—Discussion of Examples 1 Through 8

In this study, a nanofibrous-skin, hydrogel-core encapsulation device suitable for the safe delivery of insulin-producing cells, especially human SC-β cells, was developed. The design takes advantage of the unique, tunable nanofibrous structure of electrospun nonwoven membranes and the biocompatible, tough and easy-to-process TSPU polymer. With proper nanofiber size (<500 nm) and membrane thickness (˜100 μm), the nanofibrous skin prevented cell escape or penetration while maintaining superb mass transfer. Inside the device, the applicants used a common, easy-to-crosslink alginate hydrogel to disperse the islets and avoid cell clumping for better access to nutrients, notably oxygen (Papas et al., “Oxygenation strategies for encapsulated islet and beta cell transplants,” Adv Drug Deliver Rev 139:139-156 (2019), which is hereby incorporated by reference in its entirety). In addition, alginate hydrogel materials are immunoprotective (Omer et al., “Survival and Maturation of Microencapsulated Porcine Neonatal Pancreatic Cell Clusters Transplanted into Immunocompetent Diabetic Mice,” Diabetes 52:69-75 (2003), which is hereby incorporated by reference in its entirety), support the survival of islets via high hydration (A. S. Hoffman, “Hydrogels for biomedical applications,” Adv Drug Deliver Rev 64:18-23 (2012), which is hereby incorporated by reference in its entirety) and reduce diffusion of some antibodies (Juirez et al., “Immunological and Technical Considerations in Application of Alginate-Based Microencapsulation Systems,” Frontiers Bioeng Biotechnology 2:26 (2014), which is hereby incorporated by reference in its entirety). The whole device is soft but tough, easy to fabricate and does not require any extra internal or external support to maintain its shape, leaving a unique nanofibrous surface as the host interface.

The long-term efficacy of cell encapsulation devices is highly dependent on the biocompatibility of the material and the transplantation site. Three clinically relevant transplant sites for the NICE device were investigated: the I.P. space, the epididymal fat pad (E.F.P.) (Wang et al., “A bilaminated decellularized scaffold for islet transplantation: Structure, properties and functions in diabetic mice,” Biomaterials 138:80-90 (2017), which is hereby incorporated by reference in its entirety), and the S.C. space (Merani et al., “Optimal implantation site for pancreatic islet transplantation,” Brit J Surg 95:1449-1461 (2008), which is hereby incorporated by reference in its entirety). Subcutaneous implantation was the least invasive transplantation site but caused the thickest FBR-induced fibrotic deposition around the device; modification of the site to induce a supporting vasculature will likely be needed for future development (Pepper et al., “A prevascularized subcutaneous device-less site for islet and cellular transplantation,” Nat Biotechnol 33:518-523 (2015); Pepper et al., “Transplantation of Human Pancreatic Endoderm Cells Reverses Diabetes Post Transplantation in a Prevascularized Subcutaneous Site,” Stem Cell Rep 8:1689-1700 (2017); Song et al., “Engineering transferrable microvascular meshes for subcutaneous islet transplantation,” Nat Commun 10:4602 (2019); Bowers et al., “Engineering the Vasculature for Islet Transplantation,” Acta Biomater 95:131-151 (2019); and Vlahos et al., “Modular tissue engineering for the vascularization of subcutaneously transplanted pancreatic islets,” P Natl Acad Sci Usa 114:9337-9342 (2017), which are hereby incorporated by reference in its entirety). Implantation in the E.F.P. (as a surrogate for the omentum) would provide access to vasculature (Weaver et al., “Vasculogenic hydrogel enhances islet survival, engraftment, and function in leading extrahepatic sites,” Sci Adv 3(6):e1700184 (2017), which is hereby incorporated by reference in its entirety), but the device-tissue adhesion observed during retrieval may become a challenge for clinical applications. On the other hand, the I.P. space was accessible, allowed facile retrieval of the device without severe adhesion and caused the least cellular overgrowth. The minimal fibrotic reaction against the NICE device is attributed to both the biocompatibility of the TSPU polymer as well as the softness and nanofibrous structures of the electrospun nanomembranes. Previous studies have demonstrated that nanofibers may more closely mimic the natural extracellular matrix (ECM) compared to microfibers and provide unique contact cues to modulate macrophages toward anti-inflammatory phenotypes in vitro and in vivo (Garg et al., “Macrophage functional polarization (M1/M2) in response to varying fiber and pore dimensions of electrospun scaffolds,” Biomaterials 34:4439-4451 (2013), and Wang et al., “Overcoming foreign-body reaction through nanotopography: Biocompatibility and immunoisolation properties of a nanofibrous membrane,” Biomaterials 102:249-258 (2016), which are hereby incorporated by reference in its entirety).

In addition to good biocompatibility, the function of the islets in the encapsulation device also highly depends on mass transfer. Pores should be sufficiently small to prevent direct contact between the host immune cells and the encapsulated cells. It is difficult to measure the precise pore sizes of an electrospun nonwoven membrane, however the average pore size for the NICE device was estimated about 1 μm, which was effective in preventing the invasion of immune cells into the device in most cases, perhaps also partly due to the nonwoven, multilayer nature of the membrane. Indeed, research has been directed at modifying the structure of electrospun membranes to increase cell infiltration when that is desirable (Chen et al., “Three-Dimensional Objects Consisting of Hierarchically Assembled Nanofibers with Controlled Alignments for Regenerative Medicine,” Nano Lett 19:2059-2065 (2019), which is hereby incorporated by reference in its entirety). It is important to note that the range of pore sizes for the NICE device is much larger than pores in other previously reported devices, which are on the order of 10 to 100 nm. The relatively large pore size allows better mass transfer of nutrients, insulin and metabolic wastes. It should also be noted that alginate hydrogel core of the device likely played a role in the immunoprotection. The results with proliferative cells confirmed that the NICE device was both safe and functional, preventing cell infiltration or escape while maintaining cell viability for 5 months.

In addition to a feasible implantation site and a safe and functional device, islet source is another factor that determines the clinical success of a cell replacement therapy. Thus, in this study, the performance of the device was assessed using multiple islet sources, to mimic different clinical scenarios. Currently, both islet auto-transplantation after total pancreatectomy for the indication of pancreatitis and allotransplantation with immunosuppression for the treatment of T1D have been demonstrated clinically feasible (Robertson et al., “Prevention of Diabetes for up to 13 Years by Autoislet Transplantation After Pancreatectomy for Chronic Pancreatitis,” Diabetes 50:47-50 (2001), and Millman and Pagliuca, “Autologous Pluripotent Stem Cell-Derived β-Like Cells for Diabetes Cellular Therapy,” Diabetes 66:1111-1120 (2017), which are hereby incorporated by reference in their entirety). The NICE device protected syngeneic and allogeneic islets to correct diabetes in immunocompetent mice for up to 200 days. Although promising results were achieved with these cell sources, the search for a more ideal cell source continues. There is much research interest in xenotransplantation of porcine islets, due to donor tissue abundance, economic considerations (Omer et al., “Survival and Maturation of Microencapsulated Porcine Neonatal Pancreatic Cell Clusters Transplanted into Immunocompetent Diabetic Mice,” Diabetes 52:69-75 (2003); Krishnan et al., “Noninvasive evaluation of the vascular response to transplantation of alginate encapsulated islets using the dorsal skin-fold model,” Biomaterials 35:891-898 (2014), and Mesmaeker et al., “Increase Functional Beta Cell Mass in Subcutaneous Alginate Capsules with Porcine Prenatal Islet Cells but Loss with Human Adult Islet Cells,” Diabetes 67(12):2640-2649 (2018), which are hereby incorporated by reference in their entirety) and particularly the recent advancement of genome editing to inactivate the porcine endogenous retrovirus (Niu et al., “Inactivation of porcine endogenous retrovirus in pigs using CRISPR-Cas9,” Science 357:1303-1307 (2017), which is hereby incorporated by reference in its entirety). However, it is likely challenging for devices alone, without any immunosuppression, to protect and support the function of porcine islets in humans. Indeed, comparisons of diabetes correction results in mice using rat islets to that using mouse islets, or the functional outcome of human SC-β cells implanted in immunodeficient mice to that in immunocompetent ones, suggest that the NICE device will be better suited for allogeneic than xenogeneic transplantation.

To better understand the immunoprotective function of the device with the different islet sources, the composition of the cells surrounding the device following transplantation and the antibody concentrations in circulation, in the I.P. fluid surrounding the device and within the device were examined. Different types of immune cells surrounded the device, including CD4+ T cells, B cells, macrophages, dendritic cells and neutrophils; however, very few CD8+ T cells were present even in xenografts. This may be important because CD8+ T cells are known to be recruited in non-immunoprotected β cell transplantation and can directly destroy allogeneic islets by releasing granzyme B and perforins (Faleo et al., “Assessment of Immune Isolation of Allogeneic Mouse Pancreatic Progenitor Cells by a Macroencapsulation Device,” Transplantation 100:1211-1218 (2016), and Harper et al., “CD8 T-cell Recognition of Acquired Alloantigen Promotes Acute Allograft Rejection,” Proc National Acad Sci 112:12788-12793 (2015), which are hereby incorporated by reference in their entirety). Moreover, antibody analysis showed that there were elevated IgG and IgM concentrations and existence of alloantibodies in the serum of recipient mice, and that a stronger antibody response was found in the mice with devices encapsulating xenogeneic islets than in those with devices encapsulating syngeneic or allogeneic islets. The antibody response is not unexpected since the device can prevent direct immune recognition but small molecular weight shed antigens can still diffuse out of the device and elicit immune responses by indirect recognition. However, results showed that almost no alloantibodies were present inside the device. This is likely because the nanofiber membrane and the alginate core in combination present diffusion barriers for the relatively large antibodies. Lastly, it is expected that small molecule cytokines such as interferon gamma (IFN-γ), interleukin-1β (IL-1β), and tumor necrosis factor alpha (TNF-α), with molecular weights of 17 to 51 kDa and hydrodynamic radii of 2 to 3 nm, can likely diffuse into the device and may cause harmful effects on islets over time (Faleo et al., “Assessment of Immune Isolation of Allogeneic Mouse Pancreatic Progenitor Cells by a Macroencapsulation Device,” Transplantation 100:1211-1218 (2016); Akerfeldt et al., “Cytokine-Induced 1-Cell Death Is Independent of Endoplasmic Reticulum Stress Signaling,” Diabetes 57:3034-3044 (2008), and Soldevila et al., “Cytotoxic Effect of IFN-7 Plus TNF-α on Human Islet Cells,” J Autoimmun 4:291-306 (1991), which are hereby incorporated by reference in their entirety). Delivery of anti-inflammatory agents has been demonstrated to reduce cytokine-induced islet death (Jiang et al., “Local Release of Dexamethasone From Macroporous Scaffolds Accelerates Islet Transplant Engraftment by Promotion of Anti-inflammatory M2 Macrophages, Biomaterials 114:71-81 (2017), and Dang et al., “Enhanced Function of Immuno-isolated Islets in Diabetes Therapy by Co-encapsulation With an Anti-inflammatory Drug,” Biomaterials 34:5792-801 (2013), which are hereby incorporated by reference in their entirety). Nevertheless, these immunological analyses confirm the immunoprotective function of the NICE device as supported by the functional data with multiple islet sources.

Human SC-β cells are one of the most promising alternative cell sources for diabetes cell replacement therapy. A critical challenge, however, is the delivery of these cells, ideally in a safe and retrievable device without immunosuppression. In particular, since the auto-immune disease process that originally destroyed β cells in the pancreas may be re-invigorated by the introduction of new insulin producing cells (Wen et al., “Innate Immunity and Intestinal Microbiota in the Development of Type 1 Diabetes,” Nature 455:1109 (2008), which is hereby incorporated by reference in its entirety), methods including development of safe encapsulation devices to prevent both allo- and auto-immunity without systemic immunosuppression are needed (Ernst et al., “Islet Encapsulation,” J Mater Chem B 6:6705-6722 (2018); Desai and Shea, “Advances in Islet Encapsulation Technologies,” Nat Rev Drug Discov 16(5):338-350 (2017); E. Dolgin, “Diabetes: Encapsulating the Problem,” Nature 540(7632):S60-S62 (2016); E. Dolgin, “Encapsulate This,” Nat Med 20(1):9-11 (2014); Orive et al., “Reply to Encapsulate This: The Do's and Don'ts,” Nat Med 20(1):9-11 (2014); Fuchs et al., “Hydrogels in Emerging Technologies for Type 1 Diabetes,” Chem Rev 121(18):11458-1152 (2020); Ernst et al., “Nanotechnology in Cell Replacement Therapies for Type 1 Diabetes,” Adv Drug Deliver Rev 139:116-138 (2019), and Desai and Tang, “Islet Encapsulation Therapy—Racing Towards the Finish Line?,” Nat Rev Endocrinol 14:630-632 (2018), which are hereby incorporated by reference in their entirety). The NICE device was effective in supporting the function of relatively mature human SC-B cells and reversing diabetes in both immunodeficient mice (for up to 120 days) and immunocompetent mice (for up to 60 days). It is also noted that diabetes correction occurred within a week after transplantation.

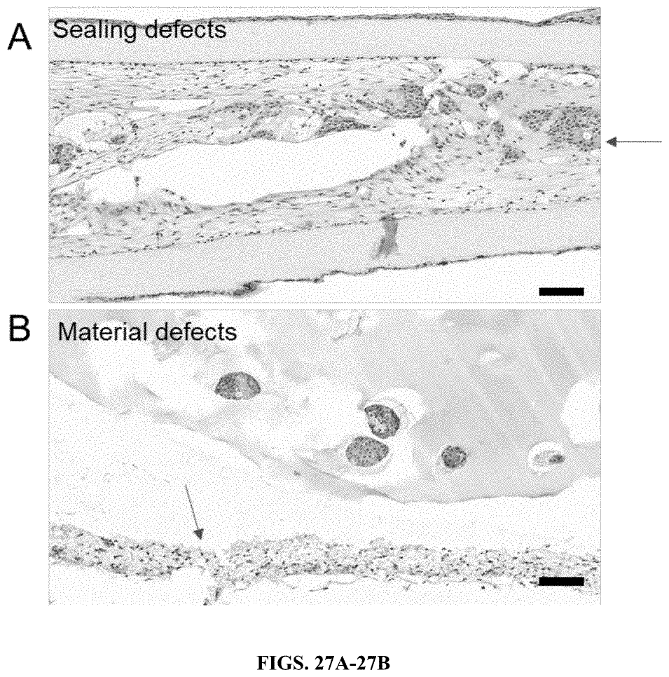

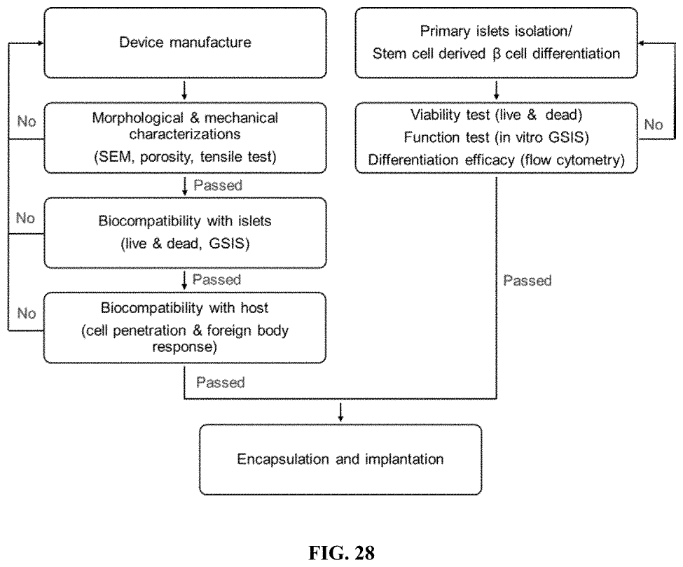

Limitations exist in this study. For example, early failures in engraftments were observed and might be due to factors including sealing defects (FIG. 27A), material defects (FIG. 27B), unintentional inconsistencies in encapsulation and surgical procedures, number and quality of islets, and biological variations of recipients. Quality control of cells and devices will be critical to minimizing early failures and to eventually making the cell encapsulation a clinical reality. More specifically, quality control strategies include standardizing the fabrication and examining representative devices from every batch including scanning electron microscopy, tensile test, porosity measurement, in vitro biocompatibility with islets and in vivo biocompatibility; only after passing these assessments could that batch of devices be used in islet and β cell encapsulation (FIG. 28). Another limitation is the lack of ideal animal models used in the study. The true potential of the NICE device for human SC-β cell-based T1D therapy would be best tested in human patients in an allogeneic setting. As a preclinical proof of concept, the applicants tested the scalability, safety and procedural feasibility (i.e. handling, implantation and retrieval) of the device with human SC-β cells in healthy dogs. Despite the challenging xenogeneic environment and use of a short, 2-week transplantation, the applicants observed viable and functional SC-β cells in retrieved devices and the device was safe, relatively easy to implant and completely retrievable using minimally invasive laparoscopic procedures. Longer-term implantation will likely lead to more tissue adhesions, rendering retrieval less smooth. The fibrotic cellular overgrowth after long-term implantation in large animal models or human patients is another major challenge for clinical applications. Given the mouse studies, which suggest that the NICE device supported long-term function of allogeneic cells even in presence of cellular overgrowth, it remains to be investigated what amount of fibrotic deposition may be permissible for cellular function in higher order animals or human patients and whether additional fibrosis-mitigating coatings (N. Bray, “Biomaterials: Modified alginates provide a long-term disguise against the foreign body response,” Nat Rev Drug Discov 15(3):158 (2016), which is hereby incorporated by reference in its entirety) may be applicable to improve device function.

In conclusion, rapid advances in stem cell technology in recent years (Pagliuca et al., “Generation of Functional Human Pancreatic 3 Cells In Vitro,” Cell 159:428-439 (2014); Rezania et al., “Reversal of diabetes with insulin-producing cells derived in vitro from human pluripotent stem cells,” Nat Biotechnol 32:1121 (2014); Millman et al., “Generation of stem cell-derived β-cells from patients with type 1 diabetes,” Nat Commun 7:11463 (2016); Velazco-Cruz et al., “Acquisition of Dynamic Function in Human Stem Cell-Derived β Cells,” Stem Cell Rep Stem Cell Rep 12:012 (2019), and Nair et al., “Recapitulating endocrine cell clustering in culture promotes maturation of human stem-cell-derived β cells,” Nat Cell Biol 21:263-274 (2019), which are hereby incorporated by reference in their entirety) have fueled hope that SC-β cells may be used in patients with diabetes to functionally cure T1D. To enable these therapies, encapsulation represents a promising, immunosuppression-free method to protect the cells from the auto-immune and allo-immune attack while mitigating potential risks such as teratoma formation by undifferentiated stem cells (An et al., “Designing a retrievable and scalable cell encapsulation device for potential treatment of type 1 diabetes,” Proc National Acad Sci 115:E263-E272 (2018), which is hereby incorporated by reference in its entirety). In this work, the applicant's report the NICE device as a translatable strategy in the delivery of insulin-producing cells, particularly human SC-β cells. The low complexity design, the relatively easy fabrication, and the balance of safety and function make the NICE device an ideal candidate for future development and eventual clinical applications.

### Materials and Methods of Examples 1-9

Study design: The purpose of this study was to design a scalable device to deliver insulin-producing cells, particularly human SC-β cells, for T1D treatment. Animals were handled and cared by trained scientists and approved by Cornell Institutional Animal Care and Use Committee. The applicants carried out islet encapsulation experiments using different types of islets in the I.P. space in mice to examine islet viability and function in this device and scaled up the device using human SC-β cells in healthy dogs to test its safety, retrievability and biocompatibility. Rodent islets were transplanted in immunocompetent diabetic mouse models, human islets were transplanted into immunodeficient diabetic mouse models, and human SC-β cells were transplanted into immunodeficient and immunocompetent diabetic mouse models as well as immunocompetent healthy dogs. The optimal number of islets used in each study was determined based on previous experience. Sample size, including number of mice per group, was chosen to ensure adequate power and was based on historical data. All mice used were males to eliminate any potential confounding influences of gender differences. All mice were randomly assigned to treatment groups, and all analyses were performed blinded to treatment conditions. No animals were excluded from analysis, and no outliers were excluded. The number of biologic replicates is specified in the figure legends.

Chemicals: Calcium chloride (CaCl2)), barium chloride (BaCl2), sodium chloride (NaCl), potassium chloride (KCl), tetrahydrofuran (THF) and N, N-Dimethylformamide (DMF) were purchased from Sigma-Aldrich Co. (St. Louis, Mo.). Glucose was purchased from Mallinckrodt Pharmaceuticals (Dublin, Ireland). Ultrapure, sterile sodium alginate (SLG100) was purchased from FMC BioPolymer Co. (Philadelphia, Pa.). Thermoplastic silicone polycarbonate urethane (TSPU), a product of DSM Biomedical (CarboSil, Exton, Pa., USA) was received as a gift.

Animals: 8 weeks-old male C57BL/6, BALB/c, FVB-Tg(CAG-luc,-GFP)L2G85Chco/J (L2G85) and NOD.Cg-Prkdcscid Il2rgtm1Wjl/SzJ (NSG) mice were purchased from The Jackson Laboratory (Bar Harbor, Me.). SCID-Beige mice were obtained from Taconic Farms (Hudson, N.Y.). Sprague-Dawley rats were obtained from Charles River Laboratories (Wilmington, Mass.). Human islets were provided by Prof. Yong Wang from Division of Transplant Surgery, University of Virginia, Prof. Chengyang Liu and Prof. Ali Naji from Department of Surgery, Hospital of the University of Pennsylvania. Beagle dogs were obtained from Marshall Bioresources (Clyde, N.Y.). All animal procedures were approved by the Cornell Institutional Animal Care and Use Committee.

Stem cell differentiation: 18×106 viable HUES8 human embryonic stem cells (hESC) were seeded into a 30-mL spinner flask. The cells were cultured in mTeSR1 for 72 hr and then differentiation initiated. The table (Table 1) detailed duration, basal media, and added supplements for each stage of differentiation. On the first day of stage 6, the cells were resized by single cell dispersion with TrypLE at 37° C. The cells were then replanted in S6 media, replenished every other day, and placed on an orbital shaker (Benchmark) set at 100 revolutions per minute (rpm) in 6 well plates (Pagliuca et al., “Generation of Functional Human Pancreatic β Cells In Vitro,” Cell 159:428-439 (2014), and Velazco-Cruz et al., “Acquisition of Dynamic Function in Human Stem Cell-Derived β Cells,” Stem Cell Rep Stem Cell Rep 12:012 (2019), which are hereby incorporated by reference in their entirety).

TABLE 1
Stem cell differentiation protocol.

| | Duration | | |
|---|---|---|---|
| Stage | (days) | Media Formulation | Added Supplements |
| 1 | 3 | 500 mL MCDB 131 (Cellgro; 15-100-CV) | 100 ng/mL Activin A (R&D |
| | | supplemented with 0.22 g glucose (MilliporeSigma; | Systems; 338-AC) + 3 μM |
| | | G7528), 1.23 g sodium bicarbonate (MilliporeSigma; | Chir99021 (Stemgent; 04-0004-10) |
| | | S3817), 10 g bovine serum albumin (BSA) (Proliant; | for 24 h |
| | | 68700), 10 μL ITS-X (Invitrogen; 51500056), 5 mL | |
| | | GlutaMAX (Invitrogen; 35050079), 22 mg vitamin C | |
| | | (MilliporeSigma; A4544), and 5 mL | |
| | | penicillin/streptomycin (P/S) solution (Cellgro; 30-002- | |
| | | CI) | |
| 2 | 3 | 500 mL MCDB 131 supplemented with 0.22 g glucose, | 50 ng/ml KGF (Peprotech; AF- |
| | | 0.615 g sodium bicarbonate, 10 g BSA, 10 μL ITS-X, 5 | 100-19) |
| | | mL GlutaMAX, 22 mg vitamin C, and 5 mL P/S | |
| 3 | 1 | 500 mL MCDB 131 supplemented with 0.22 g glucose, | 50 ng/ml KGF + 200 nM |
| | | 0.615 g sodium bicarbonate, 10 g BSA, 2.5 mL ITS-X, | LDN193189 (Reprocell; 040074) + |
| | | 5 mL GlutaMAX, 22 mg vitamin C, and 5 mL P/S | 500 nM PdBU (MilliporeSigma; |
| | | | 524390) + 2 μM Retinoic Acid |
| | | | (MilliporeSigma; R2625) + 0.25 |
| | | | μM Sant1 (MilliporeSigma; |
| | | | S4572) + 10 μM Y27632 |
| 4 | 5 | 500 mL MCDB 131 supplemented with 0.22 g glucose, | 5 ng/mL Activin A + 50 ng/mL |
| | | 0.877 g sodium bicarbonate, 10 g BSA, 2.5 mL ITS-X, | KGF + 0.1 μM Retinoic Acid + |
| | | 5 mL GlutaMAX, and 22 mg vitamin C | 0.25 μM SANT1 + 10 μM Y27632 |
| 5 | 7 | 500 mL MCDB 131 supplemented with 1.8 g glucose, | 10 μM ALK5i II (Enzo Life |
| | | 0.877 g sodium bicarbonate, 10 g BSA, 2.5 mL ITS-X, | Sciences; ALX-270-445-M005) + |
| | | 5 mL GlutaMAX, 22 mg vitamin C, 5 mL P/S, and 5 | 20 ng/mL Betacellulin (R&D |
| | | mg heparin (MilliporeSigma; 9041-08-01) | Systems; 261-CE-050) + 0.1 μM |
| | | | Retinoic Acid + 0.25 μM SANT1 + |
| | | | 1 μM T3 (Biosciences; 64245) + |
| | | | 1 μM XXI (MilliporeSigma; |
| | | | 595790) |
| 6 | 12-20 | 500 mL MCDB 131 supplemented with 0.23 g glucose, | — |
| | | 10.5 g BSA, 5.2 mL GlutaMAX, 5.2 mL P/S, 5 mg | |
| | | heparin, 5.2 mL MEM nonessential amino acids | |
| | | (Corning; MT25025Cl), 84 μg ZnSO4 | |
| | | (MilliporeSigma; 10883), 523 μL Trace Elements A | |
| | | (Corning; 25-021-CI), and 523 μL Trace Elements B | |
| | | (Corning; 25-022-CI) | |

Flow cytometry: Clusters were single cell dispersed with TrypLE in a water bath at 37° C. The cells were washed with PBS once and then fixed with 4% paraformaldehyde overnight at 4° C. The cells were blocked with staining buffer [5% donkey serum (Jackson Immunoresearch; 017-000-121) and 0.1% Triton-X 100 (Acros Organics; 327371000) in PBS)], incubated overnight at 4° C. with primary antibodies (Table 2), washed with staining buffer, stained with secondary antibodies (Table 2) for 2 hr at room temperature, and resuspended in staining buffer to analyze on a LSRII (BD Biosciences). FlowJo was used to generate dot plots and percentages.

TABLE 2
List of antibodies used in analyzing SC-β cells by flow cytometry.

| Type | Antibody | Dilution | Company | Part # |
|---|---|---|---|---|
| primary | rat anti-C-peptide | 1:300 | DSHB | GN-ID4-S |
| primary | mouse anti-glucagon | 1:300 | Abcam | ab82270 |
| primary | mouse anti-NKX6.1 | 1:100 | DSHB | F55A12-S |
| secondary | anti-rat-AF488 | 1:300 | Invitrogen | A21208 |
| secondary | anti-rat-PE | 1:300 | Jackson | 712-116- |
| | | | Immunoresearch | 153 |
| secondary | anti-mouse-AF594 | 1:300 | Invitrogen | A21203 |
| secondary | anti-mouse-AF647 | 1:300 | Invitrogen | A31571 |

Device fabrication: 8%, 10%, 12% and 14% (w/v) TSPU solutions were prepared by dissolving TSPU in a mixture of THE and DMF (3/2). The custom-built electrospinning device was equipped with a syringe pump (Harvard Apparatus, MA), a 10-mL syringe, a stainless-steel blunt needle, a constantly moving needle holder, a rotating collector and a high voltage supply (Gamma High Voltage, Ormond Beach, Fla.). The nanofibers were spun at 13 kV with a pumping rate of 0.5 mL/h and with a 23 G blunt needle as the spinneret. The spinneret was mounted on a sliding table moving back and forth with a speed of 5 cm/s. Working distance was fixed at 10 cm. A rotating target (i.e. stainless-steel rods with diameters ranging from 0.5 mm to 3 mm) was placed in the path of the polymer solution jet. The rod was connected to an AV motor controlled by rheostat (VWR) and rotated at 400-500 rpm. The rod template was pre-coated with a thin layer of sucrose. The nanofibrous tubes were removed from the template by soaking in water. The tubes were cut into desired length and sealed at one end by hand impulse sealer (Impulse Sealer Supply, CA). The devices were soaked in 70% ethanol and sterilized using ultraviolet (UV) light for further study.

Isolation of rodent pancreatic islets: Mouse pancreatic islets were isolated from 8-week-old male C57BL/6 mice, BALB/c mice or L2G85 mice. For mice, one bottle of Collagenase (CIzyme RI 005-1030, Vitacyte, Indiana, USA) was reconstituted in 30 mL M199 media (Gibco, USA). The bile duct was cannulated with a 27 G needle as described previously (Wang et al., “Scaffold-supported Transplantation of Islets in the Epididymal Fat Pad of Diabetic Mice,” J Vis Exp 125:54995 (2017), which is hereby incorporated by reference in its entirety) and the pancreas was distended with cold Collagenase. The perfused pancreases were then removed and digested in a 37° C. water bath for 17 min. Sprague Dawley rats from Charles River Laboratories weighing approximately 300 g were used for harvesting islets. For rat islet isolation, one bottle of Liberase (Research Grade, Roche) was reconstituted in 33 mL M199 media (Gibco, USA). Briefly, the bile duct was cannulated, and the pancreas was distended by an injection of Liberase solution. The pancreas was digested in a 37° C. water bath for 28 min. The purification of rodent islets was detailed in Supplementary Materials. Purified islets were hand-counted by aliquot under a stereomicroscope (Olympus SZ61). Islets were then cultured in RPMI 1640 media with 10% fetal bovine serum (FBS) and 1% penicillin/streptomycin (P/S) for further use.

Isolation of human pancreatic islets: The pancreatic organs were obtained from the organ procurement organization under the United Network for Organ Sharing (UNOS). The islets were isolated in the Human Islet Core at the University of Pennsylvania following the guidelines of Clinical Islet Transplantation (CIT) consortium protocols (Ricordi et al., “National Institutes of Health-Sponsored Clinical Islet Transplantation Consortium Phase 3 Trial: Manufacture of a Complex Cellular Product at Eight Processing Facilities,” Diabetes 65:3418-3428 (2016), which is hereby incorporated by reference in its entirety). Briefly, the pancreas was digested following intraductal injection of collagenase and neutral protease enzyme in Hanks' balanced salt solution (HBSS). Liberated islets were then purified on continuous density gradients (Cellgro/Mediatech) using the COBE 2991 centrifuge, cultured in CIT culture media and kept in a humidified 5% CO₂ incubator.

Cell encapsulation for mouse study: For a typical mouse study, the electrospun nanofiber device with a diameter of 1 mm was cut into 2.5 cm lengths. Immediately prior to encapsulation, the cultured islets (300-350 IEQ) were centrifuged at 1000 rpm for 1 min and all supernatant was aspirated. 20 μl 2% (w/v) solution of SLG100 alginate dissolved in 0.9% (w/v) NaCl solution was added into the islets pellet. The cell-loaded alginate solution was filled into the device. For human islets, ˜800 IEQ were collected and loaded with alginate as described above. For SC-β cells, ˜1250 clusters were used. The device was further submerged in a crosslinking buffer containing 100 mM CaCl₂) and 5 mM BaCl₂. The device was taken out and extra solution on the surface of the device was removed with autoclaved tissue. The end of the tube was thermally sealed with hand impulse sealer (Impulse Sealer Supply). The device was washed with 0.9% (w/v) NaCl solution and ready to use.

Bioluminescence imaging: The GFP/luciferase expressing aggregates were collected and centrifuged at 1000 rpm for 3 min. The cells were washed with saline and centrifuged again to form a pellet. Then 300-400 GFP/luciferase MSC or 4T1 spheroids were loaded with alginate into the NICE device as described above. The GFP/luciferase MSCs- or 4T1-laden devices were implanted into healthy C57BL/6 mice. The GFP/luciferase islet-laden device was prepared as described above and implanted into healthy C57BL/6 mice. The mice were injected with 150 mg/kg body weight luciferin (#122799, PerkinElmer) and imaged with IVIS Spectrum System (PerkinElmer) at the Biotechnology Resource Center at Cornell.

Transplantation and retrieval of the device in mice: For diabetes study, immunocompetent male C57BL/6 or immunodeficient male SCID-beige or male NSG mice were utilized for transplantation. To create diabetic mice, mice were injected I.P. with freshly prepared streptozotocin (STZ, Sigma Aldrich, 130 mg/kg body weight) solution (13 mg/mL in 5 mM sodium citrate buffer solution). Only mice whose non-fasted blood glucose concentrations were above 300 mg/dL with two consecutive measurements were considered diabetic and underwent transplantation. Detailed procedure of transplantation can be found in supplementary materials. Glucose was monitored twice a week. A small drop of blood was collected from the tail vein using a lancet and tested using a commercial glucometer (Contour next, Ascensia Diabetes Care, NJ). Mice changing from a diabetic state (blood glucose>˜350 mg/dl for at least two consecutive measurements) to a non-diabetic state (blood glucose<˜200 μmg/dl for at least two consecutive measurements) were considered as engrafted mice. For all the engrafted mice, devices were explanted in a survival surgery after a certain time and the mice remained alive after retrieval. Blood glucose was monitored after explanting the device to further confirm the function of the device. Some retrieved samples were examined by ex vivo GSIS test (described in Supplementary Materials) and fixed in 10% formalin for further staining. For the other devices, the nanofibrous skin was peeled off and the islet-laden alginate inside the device was imaged using the stereomicroscope.

IPGTT: Mice were fasted overnight before receiving an intraperitoneal glucose bolus (2 g/kg body weight). The healthy mice and diabetic mice were used as positive control and negative control, respectively. Blood glucose was monitored at regular intervals (time 0, 5, 15, 30, 60, 90, and 120 min) after injection, allowing for the AUC to be calculated and analyzed between groups.

In vivo GSIS: The SCID-beige mice implanted with human islet-laden devices or NSG mice implanted with SC-β cells were fasted overnight before receiving an intraperitoneal glucose bolus (2 g/kg body weight). Blood was collected from the orbital vein before injection and 90 min after injection using a tube with clotting activator (Microvette 300 Z, Sarstedt). The tubes were then centrifuged at 2000 relative centrifugal force (rcf) for 10 min. Serum was collected from the tube and frozen for further analysis. Human C-peptide concentration in the serum was detected using ultra-sensitive human C-peptide ELISA kit (Mercodia).

Cellular analysis of fibrotic layer by flow cytometry: After the engrafted devices were retrieved from the recipients, the fibrotic layer surrounding the encapsulation device was carefully peeled off using tweezers. The fibrotic layer was cut into small pieces and digested with 1 mg/ml Type I collagenase (Worthington Biochemical Corporation; LS004194) for 1 h in incubator. The digestion was stopped by adding cell culture medium containing 10% FBS. The cell solution was filtered through Falcon 40 μm strainer (Corning; 431750) to get single cell solution. Cells were centrifuged at 1000 rpm for 5 min. Supernatant was discard and cells were washed with PBS solution to remove the remaining FBS. The cells were stained with Zombie Yellow Fixable Viability Kit (BioLegend; 423103) following the manufacturer's instructions. Cells were washed one time with 2 ml Cell Staining Buffer (BioLegend; 420201) and centrifuged into a pellet. Fc receptors were blocked by pre-incubating cells with TruStain FcX PLUS (anti-mouse CD16/32) Antibody (BioLegend; 156603) in 100 μl Cell Staining Buffer for 5 min on ice. Then cells were labeled with mixed antibodies (Table 3) on ice for 15 min. The cells were washed twice with 2 ml of Cell Staining Buffer by centrifugation at 350 rcf for 5 min. The cell pellet was resuspended in 0.5 ml of Cell Staining Buffer. UltraComp eBeads Compensation Beads (Thermofisher; 01-2222-41) incubated with each antibody following manufactures' instruction were used for compensation. Finally, stained cells were analyzed using Attune NxT flow cytometer (Thermo Fisher). The data were analyzed by FlowJo software v10.7.

TABLE 3
List of antibodies used in analyzing composition
of fibrotic layer by flow cytometry.

| Antibody | Color | Dilution | Company | Part # |
|---|---|---|---|---|
| anti-mouse Ly-6G/ | AF488 | 1:100 | BioLegend | 108419 |
| Ly-6C (Gr-1) | | | | |
| anti-mouse CD45 | PerCP | 1:100 | BioLegend | 103129 |
| anti-mouse CD11c | APC | 1:100 | BioLegend | 117309 |
| anti-mouse/human | AF700 | 1:100 | BioLegend | 103231 |
| CD45R/B220 | | | | |
| anti-mouse/human | APC/Cyanine7 | 1:100 | BioLegend | 101225 |
| CD11b | | | | |
| anti-mouse CD3 | Pacific Blue | 1:100 | BioLegend | 100213 |
| anti-mouse F4/80 | PE | 1:100 | BioLegend | 123109 |
| anti-mouse CD4 | AF594 | 1:100 | BioLegend | 100446 |
| anti-mouse CD8a | PE/Cyanine7 | 1:100 | BioLegend | 100721 |

Cell encapsulation for dog experiment: Three devices about 17 cm long were prepared and sterilized as mentioned above. 2500 clusters SC-β cells were suspended in 140 μl 2% SLG100 solution and loaded into one device using PE50 tubing. The device was crosslinked and sealed as mentioned above. The devices were submerged in 0.9% saline and ready for implantation.

Laparoscopic implantation and retrieval in dogs: Dogs were premedicated with glycopyrrolate and butorphanol, induced with propofol, and anesthetized with isoflurane and oxygen. The abdomen was clipped and prepared for sterile surgery. A 10-mm laparoscopic camera port and two 5-mm instrument ports were percutaneously inserted into the abdomen. The abdomen was insufflated to 12 mm Hg pressure with CO₂. The device in a 10 mL pipette was inserted into the abdomen through the left-side instrument port. Then, the device was flushed out from the pipette using sterile saline. A laparoscopic probe was introduced through the right-sided 5-mm port and was used to manipulate the device so that it was placed between the liver and the diaphragm. The remaining ports were then removed, and the port sites were closed with 3-0 polydioxanone suture material. For retrieval of the devices, the procedure was similar using one 10-mm camera port and one or two 5-mm instrument ports. The previously implanted device was located and photographed. The device was grasped with laparoscopic Kelly forceps.

Histological analysis: The implants were harvested from the mice and fixed in 10% formalin, dehydrated with graded ethanol solutions, embedded in paraffin, and sectioned by Cornell Histology Core Facility. The samples were sliced on a microtome at a thickness of 5 μm. The sections were stained with H&E and then imaged by a microscope (IN200TC, Amscope). To conduct immunofluorescent staining, the histological slides were deparaffinized followed by antigen retrieval as described before (Wang et al., “A bilaminated decellularized scaffold for islet transplantation: Structure, properties and functions in diabetic mice,” Biomaterials 138:80-90 (2017), which is hereby incorporated by reference in its entirety). Non-specific binding was blocked via incubation with 5% donkey serum (S30-M, Sigma) for 1 h at room temperature. Sections were decanted and incubated with primary antibodies overnight at 4° C. The sections were then washed and incubated with the fluorescence-conjugated secondary antibodies for 1 h at room temperature. Nuclei were labeled with DAPI and slides were covered with fluorescent mounting medium (F6057, Sigma). Finally, the sections were imaged through confocal microscopy (FV1000, Olympus, Japan). The antibodies used here were listed in Table 4. The thickness of fibrotic layer, the density of α-SMA+ cells and the percentage of hormone-expressing cells were analyzed using ImageJ software.

TABLE 4
List of antibodies used in immunofluorescent staining.

| Type | Antibody | Dilution | Company | Part # |
|---|---|---|---|---|
| primary | rat anti-C-peptide | 1:100 | DSHB | GN-ID4 |
| primary | mouse anti-NKX6.1 | 1:300 | DSHB | F55A12 |
| primary | rabbit anti-alpha | 1:200 | Abcam | ab5694 |
| | smooth muscle actin | | | |
| | antibody | | | |
| primary | rabbit anti-insulin | 1:200 | Abcam | ab63820 |
| primary | mouse anti-glucagon | 1:200 | Sigma | G2654 |
| primary | goat anti-GFP | 1:200 | Rockland | 600-101- |
| | | | | 215S |
| primary | rabbit anti-MAFA | 1:200 | LifeSpan | LP9872 |
| | | | Bioscience | |
| primary | rabbit anti-CD3 | 1:100 | Abcam | ab5690 |
| primary | rat anti-F4/80 | 1:50 | ThermoFisher | 14-4801-82 |
| secondary | anti-rat-AF555 | 1:400 | ThermoFisher | A11055 |
| secondary | anti-mouse-AF488 | 1:400 | ThermoFisher | A21202 |
| secondary | anti-rabbit-AF594 | 1:400 | ThermoFisher | A11037 |
| secondary | anti-goat-AF488 | 1:400 | ThermoFisher | A11055 |
| secondary | anti-rabbit-AF568 | 1:400 | ThermoFisher | A10042 |
| secondary | anti-rat-AF488 | 1:400 | ThermoFisher | A21208 |

Stem cell culture: The nondiabetic HUES8 human embryonic stem cell (hESC) line was used to generate stem cell derived § (SC-β) cells in this study. Undifferentiated hESCs were cultured in 30-mL spinner flasks (Reprocell; ABBWVS03A) on a rotator spinning plate (Chemglass) at 60 revolutions per minute (rpm) in mTeSR1 media (StemCell Technologies; 05850). The flask remained in a humidified 5% CO₂ incubator at 37° C. The cells were passaged every 3 days by single cell dispersion with Accutase (StemCell Technologies; 07920) and counted with a Vi-Cell XR (Beckman Coulter). 18×106 viable cells were seeded for propagation in mTeSR1 with 10 μM Y27632 (Abcam; ab120129).

Static glucose stimulated insulin secretion (GSIS) of SC-β cells: Clusters were collected and washed twice in Krebs Ringer Buffer (KRB), made from 128 mM NaCl, 5 mM KCl, 2.7 mM CaCl2), 1.2 mM MgSO4, 1 mM Na2HPO4, 1.2 mM KH2PO4, 5 mM NaHCO₃, 10 mM HEPES (Gibco; 15630-080), and 0.1% BSA. The clusters were incubated in transwells (Corning; 431752) in a 24-well plate at 2 mM glucose KRB for a 1-hour equilibration. The transwells were drained and transferred to 2 mM glucose and then 20 mM glucose for 1-hour incubations. Supernatant was collected and insulin secretion was quantified with a Human Insulin Elisa kit (ALPCO; 80-INSHU-E10.1). The cells were then single cell dispersed with TrypLE and viable cells counted on a Vi-Cell XR to normalize insulin secretion.

Dynamic GSIS of SC-β cells: A dynamic GSIS system was built with an 8-channel dispenser pump (ISMATEC; ISM931C) connected to 38.1 mm inlet and outlet two-stop tubing (ISMATEC; 070602-04i-ND) with a 275-μl cell chamber (BioRep; Peri-Chamber) in between and a dispensing nozzle (BioRep; PERI-NOZZLE) attached at the end through 101.6 mm connection tubing (BioRep; Peri-TUB-040). The system was maintained in a water bath at 37° C. Clusters were washed twice in KRB buffer and loaded into the cell chamber between Bio-Gel P-4 polyacrylamide beads (Bio-Rad; 150-4124). The chamber was perfused with 2 mM glucose KRB for 90 minutes prior to collecting effluent for 8 minutes. Next, the clusters were challenged with 20 mM glucose KRB for 24 minutes and then with 2 mM KRB for additional 12 minutes. The flow rate is 100 μL/min with 2-4 min collections. Last, clusters within the cell chamber were lysed in 10 mM TRIS (MilliporeSigma; T6066), 0.2% Triton-X 100 solution, and 1 mM EDTA. DNA from the lysed solution and insulin secretion were quantified with Quant-iT Picogreen dsDNA (Invitrogen; P7589) and Human Insulin Elisa kits, respectively.

Oxygen consumption rate (OCR): 15-30 clusters of primary human islets or SC-β cells were placed in XF24 islet capture microplates (Agilent; 101122-100) with Seahorse media (7.4 pH RPMI-1640 (Sigma; R6504)) at 20 mM glucose. The Seahorse XFe24 flux analyzer (Agilent) measured OCR of the clusters. Measurements were normalized to DNA through Quant-iT Picogreen dsDNA assay kit (Invitrogen; P7589).

Characterizations of device: To observe the morphology of nanofibers, membrane samples were gold sputter-coated and examined by the scanning electron microscope (SEM) (LEO 1550 FESEM). ImageJ (NIH, Bethesda, USA) was applied to quantitatively characterize the fiber diameter. 10 random fibers were selected and measured in each image, and a total of 10 images were counted. To study the tensile strength of the electro-spun fibers, the tube was mounted on the dynamic mechanical analysis instrument (DMA Q800) with a distance of ˜1.5 cm between the holders. The tensile testing was conducted at a rate of 0.5 N/min at room temperature. Stress (MPa) and strain (%) were automatically calculated by the software. The Young's modulus was obtained by measuring the slope of the stress-strain curve in the elastic region between 10% and 20%. Four samples were tested for the experiment.

Generation of green fluorescent protein (GFP)/luciferase-expressing cell line: Plasmid containing enhanced green fluorescent protein (EGFP) gene (720 bp) and humanized firefly luciferase (Luc2) gene (1653 bp) was constructed by Vector Builder. 293T cell line and 4T1 cell line were received as gifts. Strain C57BL/6 Mouse Mesenchymal Stem Cells (MSCs) (MUBMX-01001) were purchased from Cyagen (Santa Clara, Calif.). NIT-1 cells (ATCC CRL-2055) were purchased from ATCC. 293T cells were cultured in DMEM (#2051526, Gibco) supplemented with 10% FBS and 1% P/S. 4T1 cells were cultured in RPMI 1640 media with 10% FBS and 1% P/S. Mesenchymal stem cell (MSC) lines were cultured in Mesenchymal Stem Cell Growth Medium (GUXMX-90011, Cyagen). NIT-1 cells were cultured in F-12K medium with 10% FBS and 1% P/S. Lentivirus vectors were produced by transfecting 293T cells with the designed plasmid using ViraPower Bsd Lentiviral Support Kit (K497000, ThermoFisher). Media containing lentivirus vectors were collected and stored at 4° C. MSC single cell solutions were prepared and 3 mL media containing lentivirus vectors were used to infect MSCs in 6-well plate. After 48 h of infection, lentivirus medium was discarded, and fresh MSC growth medium was added. GFP+/luciferase+ MSCs were verified under a fluorescent microscope. For the formation of MSC spheroids, about 2.5 mL solution containing 1 million MSCs were added in a non-adherent 25 mm2 petri dish. Then the cells were cultured on an orbital shaker with a speed of 50 rpm overnight. The spheroids were imaged with a fluorescent microscope mentioned above. The GFP/luciferase 4T1 spheroids were generated in the same manner as GFP/luciferase MSC spheroids.

Purification of rodent islets: The digestion was stopped by adding 20-25 mL of cold M199 media with 10% heat-inactivated fetal bovine serum (FBS) and a slight shaking. For both mouse and rat islets, digested pancreases were washed twice in the same M199 media, filtered through a 450 mm sieve, and then suspended in a Lymphocyte Separation Medium (LSM, Corning, 25-072-CV)/M199 media gradient and centrifuged at 1750 relative centrifugal force (rcf) for 20 min at 4° C. This gradient centrifugation step was repeated when desired for higher purity islets. Finally, the islets were collected from the gradient and further isolated by a series of gravity sedimentations, in which each supernatant was discarded after 4 min of settling.

In vitro and ex vivo GSIS of islets inside device: Islet-laden devices and the same number of free-floating islets were cultured in 2 mL RPMI 1640 complete media for 1 day or 7 days in nonadherent 25 mm2 culture dishes. After culture, the devices or free-floating islets were incubated in pre-warmed KRB solution supplemented with 25 mM HEPES, 1 mM L-glutamax, 0.1% BSA and 2.8 mM D-glucose for 30 min at 37° C., 5% CO2, and then incubated for 1 h with 2.8 mM or 16.7 mM D-glucose under the same condition. The supernatant was collected and frozen for future analysis. The insulin content in the supernatant was quantified by mouse insulin ELISA kit (ALPCO) according to the manufacturer's specifications. Absorbance of reaction solution at 450 nm was measured in the Synergy plate reader (Biotek). For devices retrieved from mice, the devices were put in KRB buffer supplemented with 2.8 mM D-glucose for 30 min and incubated in KRB buffer supplemented with 2.8 mM or 16.7 mM D-glucose for 90 min. The stimulation index (SI) was calculated as the ratio of the insulin value after high glucose (16.7 mM) stimulation divided by insulin value after low glucose (2.8 mM) solution.

Live and dead staining: Islet-laden devices and a same number of free-floating islets were cultured as described above for 1 day in nonadherent culture dish. After culture, the nanofiber skin of the device was peeled off. The islet-laden alginate inside the device and the free-floating islets were stained by calcein-AM (green, live) and ethidium homodimer (red, dead) according to the manufacture's protocol (R37601, Thermo fisher). Fluorescent microscopic images were taken using a digital inverted microscope (EVOS FL Cell Imaging System). Quantification of the percentage of live cells in islets was carried out by calculating the intensity of fluorescence using ImageJ.

Transplantation procedures in mice: The mice were anesthetized using 3% isoflurane in oxygen and maintained at the same rate throughout the procedure. The abdomens of the mice were shaved and alternately scrubbed with betadine and isopropyl alcohol to create a sterile field before being transferred to the surgical field. A ˜1 cm incision was made along the midline of the abdomen and the peritoneum was exposed using blunt dissection. The peritoneum was then grasped with forceps and a ˜1 cm incision was made. Two devices with 600-700 islet equivalent (IEQ) in total were then inserted into the peritoneal cavity through the incision. The incision was closed using 6-0 silk sutures (DemeTECH, FL). The skin was then closed over the incision using wound clips.

Generation of immunosuppressed mouse model: 50 mg/ml rapamycin (LC Laboratories) stock solution in 100% ethanol was prepared and stored in ˜80° C. The stock solution was diluted in 10 ml mixture [5 ml of 10% poly (ethylene glycol) (PEG) 400 in deionized (DI) water and 5 ml of 10% Tween 80 in DI water] to make a final concentration of 0.05 mg/ml. Diabetic recipients were injected with 0.5 mg/kg body weight in intraperitoneal (I.P.) space daily from ˜1 to 14 days post-transplantation. The immunosuppressed mice were transplanted with NICE devices encapsulating SC-β cells as described previously.

Measurement of total insulin content of the pancreas: Pancreas of the engrafted mice, diabetic mice and healthy mice were collected and homogenized. The homogenized tissue was placed into acid-ethanol (1.5% HCl in 70% ethanol), cut into small pieces using scissors and digested overnight at ˜20° C. Then the acid-ethanol extract solution was neutralized with pH 7.5 TRIS buffer. The samples were further diluted, and the insulin content was quantified as mentioned before.

In vivo biocompatibility analysis of the device: Blank (cell-free) devices were implanted in the I.P. space, epididymal fat pad (E.F.P.), ventral subcutaneous (S.C.) space or the dorsal S.C. space of healthy C57BL/6 mice. After 2 weeks or 1 month, the implants were harvested, fixed in 10% buffered formalin and embedded in paraffin. Cross-sections were analyzed as described in the Histological Analysis section.

Mouse total antibody analysis: C57BL/6 mice received NICE device implants encapsulating syngeneic, allogeneic and xenogeneic islets (rat islets or human SC-β cells) for a month. Blood was withdrawn from retro-orbital sinus using capillary at D0, 1 w, 2 w, 3 w and 4 w post-transplantation and collected in tubes with clotting activator (Sarstedt; Microvette 300 Z). The blood samples were centrifuged at 2000 rcf for 15 min. The serum was transferred in a new tube and stored at ˜80° C. After devices were retrieved from the recipients, 100 μl saline was added into the I.P. space. The I.P. fluids were collected after gently shaking the belly. The I.P. fluid was centrifuged at 1000 rpm for 5 min to remove cellular debris. The supernatant was transferred into a new tube and store at −80° C. The alginate core inside retrieved devices was collected and degraded with 100 μl 1 mg/ml alginate lyase (Sigma; A1603) and 1× Halt Protease Inhibitor Cocktail (Thermofisher; 87786) in saline in incubator for 1 h. Then the tube was centrifuged at 1000 rpm for 5 min to separate the alginate and the encapsulated islets. The supernatant was collected and stored at −80° C. Mouse total IgG (Thermofisher; 88-50400-22) and IgM (Thermofisher; 88-50470-22) were analyzed in all the samples (serum, I.P. fluids and degraded alginate) by using ELISA kit following manufacturer's instruction.

Donor specific alloantibody analysis: BALB/c mouse spleen was harvested and placed in 6 well plates. 5 ml RPMI1640 culture medium (Fisher; 11875-119) with 10% FBS was added in each dish. The spleen was smashed using a 3 ml syringe head. The spleen cell suspension was filtered through Falcon 40 μm strainer (Corning; 431750) to get single cell solution. The cells were pelleted by centrifugation at 1000 rpm for 5 min and the supernatant was aspirated. Red blood cells (RBC) were removed by adding RBC lysis buffer (BioLegend; 420301) and cells were incubated on ice for 5 min. The reaction was stopped by adding 20 ml PBS. Cells were centrifuged at 1000 rpm for 5 min and the supernatant was discarded. T cells were isolated using EasySep Mouse T Cell Isolation Kit (Stem cell technologies; 19851) following the manufacturer's instructions. 105 T cells in 70 μl cell staining buffer were incubated with 30 μl samples (serum collected from recipients with allografts at D0 and 4 w, and degraded alginate) at 4° C. for 30 min. Serum collected from sensitized mice with BALB/c mouse islets transplanted in the kidney capsule at 4 w post-transplantation was used as a positive control. After incubation, cells were washed with PBS and centrifuged at 1000 rpm for 5 min. Supernatant was removed and cells were stained with Zombie Yellow Fixable Viability Kit (BioLegend; 423103) following the manufacturer's instructions. Cells were washed one time with 2 ml Cell Staining Buffer (BioLegend; 420201) and centrifuged into a pellet. Fc receptors were blocked by pre-incubating cells with TruStain FcX PLUS (anti-mouse CD16/32) Antibody (BioLegend; 156603) in 100 μl Cell Staining Buffer for 5 min on ice. Cells were labeled with APC anti-mouse CD3 antibody (BioLegend; 100235) and FITC anti-mouse IgG Antibody (BioLegend; 406001) or FITC anti-mouse IgM antibody (Sigma; F9259) at 1:100 dilution on ice for 15 min. Cells were washed twice with 2 ml of Cell Staining Buffer by centrifugation at 1000 rpm for 5 min. Finally, stained cells were analyzed using Attune NxT flow cytometer (Thermo Fisher). The data were analyzed by FlowJo software v10.7.

Ca imaging: SC-β cells and human islets were retrieved from the engrafted devices as mentioned above. The SC-B cells or human islets were extracted as mentioned previously, shipped overnight, and cultured in S6 or human islet media (CMRLS with 10% FBS) for recovery of SC-β cells or human islets, respectively. The clusters were single-cell dispersed with TrypLE for 10 min and seeded in Matrigel-coated 96 well plates (Cellvis, 963-1.5H-N) overnight for attachment in their respective recovery media. Cells were washed with 2 mM glucose KRB and incubated with 20 μM Fluo-4 AM (Invitrogen; F14201) in 2 mM glucose for 45 min at 37° C. in incubator. Next, the cells were washed with 2 mM glucose KRB and challenged with 2 mM glucose KRB, 20 mM glucose KRB, and 20 mM glucose 30 mM KCl KRB, sequentially. Images were taken every minute using a Leica DMI4000 fluorescence microscope and calcium flux was calculated with ImageJ.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. An implantable therapeutic delivery device comprising:
a hydrogel core;
one or more therapeutic agents suspended within the hydrogel core; and
an elongated nanofibrous substrate having proximal and distal ends, said nanofiber substrate having an interior nanofiber wall defining an internal space that extends longitudinally between the proximal and distal ends of the substrate, wherein the hydrogel core comprising the one or more therapeutic agents is positioned within the internal space.

2. The device of claim 1, wherein the nanofibrous substrate comprises a polyurethane material.

3. The device of claim 2, wherein the polyurethane material is thermoplastic silicone-polycarbonate-urethane (TSPU).

4. The device of claim 1, wherein the nanofibrous substrate comprises a plurality of non-woven nanofiber layers.

5. The device of claim 1, wherein nanofibers of the nanofibrous substrate comprise a fiber diameter of less than 500 nm.

6. The device of claim 5, wherein the nanofibers of the nanofibrous substrate comprise a fiber diameter of between 200 and 500 nm.

7. The device of claim 1, wherein the interior wall of the nanofibrous substrate has an average thickness of between 10 and 1000 nm.

8. The device of claim 1, wherein the nanofibrous substrate comprises pores, said pores having an average diameter of 0.1 μm to 5 μm.

9. The device of claim 1, wherein the device has a rupture strain of ≥2.

10. The device of claim 1, wherein the device is elastically deformable up to about 5 MPa under a 0.5 strain.

11. The device of claim 1, wherein the device has an ultimate tensile strength of up to about 15 MPa under a strain of greater than 2.

12. The device of claim 1, wherein the device has a Young's modulus of about 12.6 MPa when calculated from 10-20% of its stress-strain curve.

13. The device of claim 1, wherein the one or more therapeutic agents are secreted from a preparation of cells suspended in the hydrogel core.

14. The device of claim 13, wherein the preparation of cells is a preparation of single cells or a preparation of cell aggregates.

15. The device of claim 13, wherein the preparation of cells is a preparation of primary cells or a preparation of immortalized cells.

16. The device of claim 13, wherein the preparation of cells is a preparation of mammalian cells.

17. The device of claim 13, wherein the preparation of cells is selected from the group consisting of a preparation of primate cells, rodent cells, canine cells, feline cells, equine cells, bovine cells, and porcine cells.

18. The device of claim 13, wherein the preparation of cells is a preparation of human cells.

19. The device of claim 13, wherein the preparation of cells is a preparation of stem cells or stem cell derived cells.

20. The device of claim 13, wherein the preparation of cells is a preparation of cells selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells,

21. The device of claim 1, wherein the one or more therapeutic agents is selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and any combination thereof.

22. The device of claim 21, wherein the one or more therapeutic agents is insulin and/or glucagon secreted from a preparation of cells suspended in the hydrogel core.

23. The device of claim 22, wherein the preparation of cells is a preparation of islet cells.

24. The device of claim 22, wherein the preparation of cells is a preparation of human SC-β cells.

25. The device of claim 23, wherein the preparation is a preparation of human cells, porcine cells, or rodent cells.

26. The device of claim 23, wherein the preparation of islet cells comprises an islet density of about 1×10³ to about 6×10le⁴ islet equivalents (IEQs)/mL.

27. The device of claim 13, wherein the preparation of cells comprises a cell density of about 1×10³ to about 6×10¹⁰ cells/mL.

28. The device of claim 1, wherein the hydrogel core further comprises one or more biologically active agents selected from the group consisting of a protein, peptide, antibody or antibody fragment thereof, antibody mimetic, a nucleic acid, a small molecule, a hormone, a growth factor, an angiogenic factor, a cytokine, an anti-inflammatory agent, and any combination thereof.

29. The device of claim 28, wherein the anti-inflammatory agent is selected from the group consisting of diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, and tolmetin.

30. The device of claim 1, wherein the hydrogel core comprises an alginate.

31. The device of claim 1, wherein the interior nanofiber wall of the nanofibrous substrate forms a tube, said tube having a diameter of about 0.5 mm to about 3 mm.

32. A method of delivering a therapeutic agent to a subject in need thereof, said method comprising:
implanting the implantable therapeutic delivery device according to claim 1 into the subject.

33. A method of producing a tubular nanofibrous substrate, the method comprising:
preparing a polyurethane solution in a solvent; and
electrospinning the polyurethane solution onto a rotating target to produce a tubular nanofibrous substrate.
